(19) United States
(12) Patent Application Publication
(10) Pub. No.: US 2009/0264384 A1
(43) Pub. Date: Oct. 22, 2009

(54) INDOLE, BENZIMIDAZOLE, AND BENZOLACTAM BORONIC ACID COMPOUNDS, ANALOGS THEREOF AND METHODS OF USE THEREOF

(75) Inventors: John R. Didsbury, Raleigh, NC (US); Tatyana Dyakonov, Greensboro, NC (US); Simon N. Haydar, Newton, PA (US); Michael L. Jones, Chapel Hill, NC (US); Francine F. Li, Chester Springs, PA (US); Christopher J. Markworth, Durham, NC (US); Jessy Matthew, Raleigh, NC (US); Frank J. Schoenen, Laurence, KS (US); Jan J. Scicinski, Mountain View, CA (US); David Middlemiss, Bishop's Stortford (GB); James F. Burns, Glen Ridge, NJ (US); Leonard A. Cabana, Durham, NC (US); Glenn C. Collupy, Durham, NC (US); David N. Vanvliet, Chapel Hill, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Nuada, Inc., Durham, NC (US)

(21) Appl. No.: 12/268,237

(22) Filed: Nov. 10, 2008

Related U.S. Application Data
(63) Continuation-in-part of application No. PCT/US2007/068671, filed on May 10, 2007.
Application No. PCT/US2007/068671 is a continuation-in-part of application No. 11/718,277, filed on Apr. 17, 2008.
Application No. 11/718,277 is a continuation-in-part of application No. 11/718,284, filed on Apr. 1, 2008.
Application No. 11/718,284 is a continuation-in-part of application No. 11/718,286, filed on Apr. 9, 2008.
(60) Provisional application No. 60/799,599, filed on May 10, 2006.
(60) Provisional application No. 60/624,057, filed on Nov. 1, 2004.
(60) Provisional application No. 60/623,996, filed on Nov. 1, 2004.
(60) Provisional application No. 60/624,055, filed on Nov. 1, 2004.

(51) Int. Cl. A61K 31/69; A61P 1/00; A61P 19/02; A61P 17/06; A61P 11/06; A61P 11/00; A61P 17/00; A61P 37/08

(52) U.S. Cl. 514/64

(57) ABSTRACT

Benzimidazole, indole and benzolactam boronic acid compounds, analogs thereof, and pharmaceutical formulations are described, along with methods of use thereof for inhibiting inflammatory cytokines such as tumor necrosis factor alpha (TNF-α) in a subject in need thereof.

### CROSS-REFERENCES TO RELATED APPLICATIONS

This application is a continuation-in-part of copending International PCT Patent Application No. PCT/US2007/068671, filed on May 10, 2007, which claims the benefit of U.S. Provisional Patent Application No. 60/799,599, filed on May 10, 2006; a continuation-in-part of copending U.S. patent application Ser. No. 11/718, 277, filed on Apr. 30, 2007, which is a U.S. National Phase application of International PCT Patent Application No. PCT/US2005/038853, which claims the benefit of U.S. Provisional Patent Application Ser. No. 60/624,057, filed on Nov. 1, 2004; a continuation-in-part of copending U.S. patent application Ser. No. 11/718,284, filed on Apr. 30, 2007, which is a U.S. National Phase application of International PCT Patent Application No. PCT/US2005/038854, which claims the benefit of U.S. Provisional Patent Application Ser. No. 60/623,996, filed on Nov. 1, 2004; and a continuation-in-part of copending U.S. patent application Ser. No. 11/718,286, filed on Apr. 30, 2007, which is a U.S. National Phase application of International PCT Patent Application No. PCT/US2005/039204, which claims the benefit of U.S. Provisional Patent Application Ser. No. 60/624,055, filed on Nov. 1, 2004, each of which is hereby incorporated by reference in its entirety.

### FIELD OF THE INVENTION

The present disclosure provides indole, benzimidazole, and benzolactam boronic acid compounds, analogs thereof, pharmaceutical formulations containing the same, and methods of use thereof, particularly for inhibiting an inflammatory cytokine such as TNF-α in a subject in need thereof.

### BACKGROUND OF THE INVENTION

Tumor necrosis factor α (TNF-α) is an inflammatory cytokine produced by neutrophils, activated lymphocytes, macrophages, NK cells, LAK cells, astrocytes, and others. TNF-α mediates a variety of cellular activities, including cytotoxic effects against tumor cells, activation of neutrophils, growth proliferation of normal cells, and immunoinflammatory, immunoregulatory, and antiviral responses. Unfortunately TNF-α also mediates a variety of pathological activities in diverse number of disease states. See generally U.S. Pat. No. 5,643,893 to Benson et al.; see also PCT Application WO 00/73253 to Palladino et al. Accordingly there is a need for new inhibitors of TNF-α. Several antibody based TNF-α inhibitors are commercially available. For example, HUMIRA® (adalimumab) is a recombinant human IgG1 monoclonal specific for human TNF and is administered subcutaneously. ENBREL® (etanercept) is a dimeric fusion protein consisting of the extracellular ligand-binding portion of the human 75 kilodalton (p75) tumor necrosis factor receptor (TNFR) linked to the Fc portion of human IgG1 specific for human TNF and is administered by subcutaneous injection. REMICADE™ (inflixamab) is a chimeric IgG1 monoclonal antibody specific for human TNF-α and is administered by intravenous infusion. However, these antibody based therapeutics have several disadvantages as compared to small molecules, including immunogenicity, cost and are limited to non-oral administration. Phosphodiesterase inhibitors are potent suppressors of many inflammatory cytokines. For example, phosphodiesterase 4 inhibitors can inhibit TNF-α release from macrophages, monocytes and T cells, which suggests that they could be effective in inflammatory diseases, including inflammatory bowel disease, but by a mechanism that is different from that of the antibody based TNF-α inhibitors (Banner et al. Trends in Pharmaceutical Sciences, Vol. 25. No. 8 (2004).

U.S. Pat. No. 5,643,893 to Benson et al. describes certain dihydroxyboryl alkyl purine, indole and pyrimidine derivatives that are useful as inhibitors of inflammatory cytokines. In general such inhibitors are compounds of the formula:

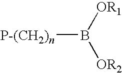

where R₁ and R₂ are both hydrogen atoms or together are a propylene chain bridging the two oxygen atoms; n is 2-6; and P is a purine, indole or pyrimidine base residue bonded via the N⁹ in the case of a purine base, or via the N¹ in the case of an indole or pyrimidine base. Certain specific substitutions, including 6- and 2,6-substituted purine derivatives, are also described.

PCT Application WO 02/085916 to Ishaq also describes certain dihydroxyboryl alkyl purine inhibitors of inflammatory cytokines of the formula:

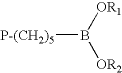

where P is a purine base, and R₁ and R₂ are both hydrogen atoms or together are a 3 to 5 carbon alkylene chain. Certain specific substitutions, including 6-, 2,6-, and 8-substituted purine derivatives, are also described (see, e.g., page 21 lines 6-7).

In spite of the foregoing there remains a need for new compounds, particularly for oral administration, for the inhibition of inflammatory cytokines such as TNF-α and methods of use thereof.

### SUMMARY OF THE INVENTION

A first aspect of the present invention is a compound of Formula I or Formula II:

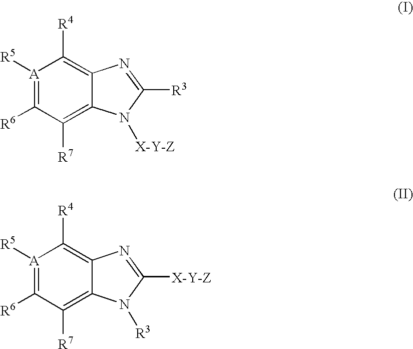

wherein:

A is N or C, subject to the proviso that R⁵ is absent when A is N;

X is —C(O)—, —S(O)₂—, or a covalent bond;

Y is linking group such as alkyl, alkenyl, cycloalkyl, alkylcycloalkyl, alkylcycloalkylalkyl, alkyloxyalkyl, aryl, alkylaryl, alkylarylalkyl, arylalkyl, cycloalkylalkyl, alkylheterocycle, heterocycloalkyl, alkylheterocycloalkyl, heterocycle, aminoalkyl, oxyalkyl, aminoaryl, oxyaryl;

Z is selected from the group consisting of —B(OR¹)OR², —CON(R¹)OR², and —N(OR¹)COR² or any of the additional alternatives for Z described in greater detail below;

R¹ and R² are each independently H, loweralkyl, or together form C₂-C₄ alkylene;

R³, R⁴, R⁵, R⁶, and R⁷ are each independently selected from the group consisting of: H, halo, loweralkyl, haloloweralkyl, haloloweralkoxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, nitro; arylalkyloxy, cycloalkyloxy, cycloalkylalkoxy, cycloalkylamino, urea, cycloalkylalkylamino, cycloalkyl, alkylcycloalkyl, hydroxyamino, alkoxyacylamino, and arylthio; and 5- or 6-membered organic rings containing 0 to 4 heteroatoms selected from the group consisting of N, O and S, which rings may be unsubstituted or substituted from 1 to 4 times with halo, loweralkyl, haloloweralkyl, haloloweralkyloxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, and nitro; and oxoheterocyclic groups; or a pharmaceutically acceptable salt or prodrug thereof (sometimes referred to as “active compounds” herein).

Another aspect of the present invention is a compound of Formula III, Formula IV or Formula V:

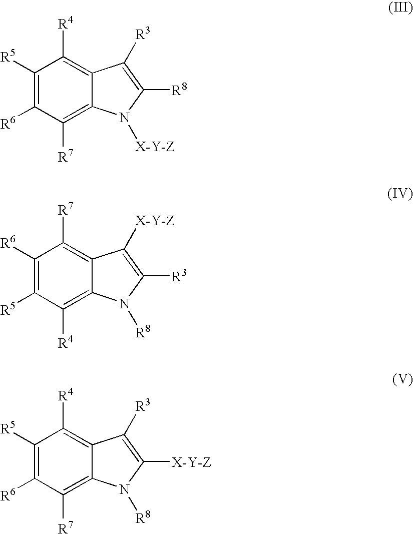

wherein:

X is —C(O)—, —S(O)₂—, or a covalent bond;

Y is alkyl, alkenyl, cycloalkyl, alkylcycloalkyl, alkylcycloalkylalkyl, alkyloxyalkyl, aryl, alkylaryl, alkylarylalkyl, arylalkyl, cycloalkylalkyl, alkylheterocycle, heterocyclealkyl, alkylheterocycloalkyl, heterocycle, aminoalkyl, oxyalkyl, aminoaryl, or oxyaryl;

Z is selected from the group consisting of —B(OR¹)OR², —CON(R¹)OR², and —N(OR¹)COR², or any of the alternatives for Z discussed below;

R¹ and R² are each independently H, loweralkyl, or together form C2-C4 alkylene; and

R³, R⁴, R⁵, R⁶, R⁷ and R⁸ are each independently selected from the group consisting of: H, halo, loweralkyl, haloloweralkyl, haloloweralkoxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, nitro arylalkyloxy, cycloalkyloxy, cycloalkylalkoxy, cycloalkylamino, urea, cycloalkylalkylamino, cycloalkyl, alkylcycloalkyl, hydroxyamino, alkoxyacylamino, and arylthio; and 5- or 6-membered organic rings containing 0 to 4 heteroatoms selected from the group consisting of N, O and S, which rings may be unsubstituted or substituted from 1 to 4 times with halo, loweralkyl, haloloweralkyl, haloloweralkyloxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, and nitro; and oxoheterocyclic groups; or a pharmaceutically acceptable salt or prodrug thereof (sometimes referred to as “active compounds” herein).

Another aspect of the present invention is a compound of Formula VI:

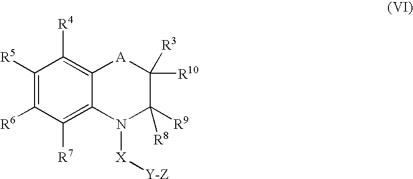

wherein:

A is S, O, SO₂ or NR;

X is —C(O)—, —S(O)₂—, or a covalent bond;

Y is alkyl, alkenyl, cycloalkyl, alkylcycloalkyl, alkylcycloalkylalkyl, alkyloxyalkyl, aryl, alkylaryl, alkylarylalkyl, arylalkyl, cycloalkylalkyl, alkylheterocycle, heterocycloalkyl, alkylheterocycloalkyl, heterocycle, aminoalkyl, oxyalkyl, aminoaryl, oxyaryl cycloalkylalkyl, alkylheterocycle, heterocycloalkyl, alkylheterocycloalkyl, heterocycle, aminoalkyl, oxyalkyl, aminoaryl, oxyaryl;

Z is selected from the group consisting of —B(OR¹)OR², —CON(R¹)OR², and —N(OR¹)COR² or any of the alternatives for Z described below;

R¹ and R² are each independently H, loweralkyl, or together form C₂-C₄ alkylene; and

R, R³, R⁴, R⁵, R⁶, R⁷, R⁸, R⁹ and R¹⁰ are each independently selected from the group consisting of: H, halo, loweralkyl, haloloweralkyl, haloloweralkoxy, loweralkoxy, hydroxy, loweralkoxycarbo, cycloalkyl, alkylcycloalkyl, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, nitro, arylalkyloxy, cycloalkyloxy, cycloalkylalkoxy, cycloalkylamino, urea, cycloalkylalkylamino, hydroxyamino, alkoxyacylamino, and arylthio; and 5- or 6-membered organic rings containing 0 to 4 heteroatoms selected from the group consisting of N, O and S, which rings may be unsubstituted or substituted from 1 to 4 times with halo, loweralkyl, haloloweralkyl, haloloweralkyloxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, and nitro; and oxoheterocyclic groups;

or R⁸ and R⁹ together are ═O or ═S; or a pharmaceutically acceptable salt or prodrug thereof.

Another aspect of the present invention is a compound of Formula VII:

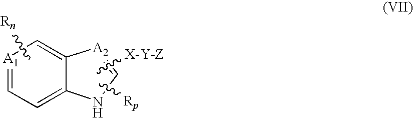

wherein:

A₁ and A₂ are each independently N or C;

X is —C(O)—, —S(O)₂—, or a covalent bond;

Y is linking group such as alkyl, alkenyl, cycloalkyl, alkylcycloalkyl, alkylcycloalkylalkyl, alkyloxyalkyl, aryl, alkylaryl, alkylarylalkyl, arylalkyl, cycloalkylalkyl, alkylheterocycle, heterocycloalkyl, alkylheterocycloalkyl, heterocycle, aminoalkyl, oxyalkyl, aminoaryl, oxyaryl;

Z is selected from the group consisting of —B(OR¹)OR², —CON(R¹)OR², and —N(OR¹)COR² or any of the additional alternatives for Z described in greater detail below;

R¹ and R² are each independently H, loweralkyl, or together form C2-C4 alkylene;

Rₙ, and Rₚ are each independently selected from the group consisting of: H, halo, loweralkyl, haloloweralkyl, haloloweralkoxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, nitro; arylalkyloxy, cycloalkyloxy, cycloalkylalkoxy, cycloalkylamino, urea, cycloalkylalkylamino, cycloalkyl, alkylcycloalkyl, hydroxyamino, alkoxyacylamino, and arylthio; and 5- or 6-membered organic rings containing 0 to 4 heteroatoms selected from the group consisting of N, O and S, which rings may be unsubstituted or substituted from 1 to 4 times with halo, loweralkyl, haloloweralkyl, haloloweralkyloxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, and nitro; and oxoheterocyclic groups; subject to the proviso that when A₁ is C, then n=1 to 4; when A₁ is N, then n=1 to 3; A₂ is C, then p=1 to 2; when A₂ is N, then n=1; or a pharmaceutically acceptable salt or prodrug thereof (sometimes referred to as “active compounds” herein).

A further aspect of the invention is a method of inhibiting tumor necrosis factor alpha in a subject in need thereof, comprising administering a compound as described above to said subject in an amount effective to inhibit tumor necrosis factor alpha.

A further aspect of the invention is a method of inhibiting phosphodiesterase in a subject in need thereof, comprising administering a compound or active agent as described herein to the subject in an amount effective to inhibit phosphodiesterase (e.g., PDE TI, PDE ITT, PDE IV, PDE V and combinations thereof such as both PDE TI and PDE IV).

A further aspect of the invention is a method of treating an inflammatory disease in a subject in need thereof, comprising administering a compound or active agent as described herein to the subject in an amount effective to treat said inflammatory disease.

A further aspect of the invention is a method of treating inflammatory bowel disease in a subject in need thereof, comprising administering a compound or active agent as described herein to the subject in an amount effective to treat inflammatory bowel disease.

A further aspect of the invention is a method of treating rheumatoid arthritis in a subject in need thereof, comprising administering a compound or active agent as described herein to the subject in an amount effective to treat rheumatoid arthritis.

A further aspect of the invention is a method of treating psoriasis in a subject in need thereof, comprising administering a compound or active agent as described herein to the subject in an amount effective to treat psoriasis.

A further aspect of the invention is a method of treating ankylosing spondylitis in a subject in need thereof, comprising administering a compound or active agent as described herein to the subject in an amount effective to treat ankylosing spondylitis.

A further aspect of the invention is a method of treating psoriatic arthritis in a subject in need thereof, comprising administering a compound or active agent as described herein to the subject in an amount effective to treat psoriatic arthritis.

A further aspect of the invention is a method of treating asthma in a subject in need thereof, comprising administering a compound or active agent as described herein to the subject in an amount effective to treat asthma.

A further aspect of the invention is a method of treating chronic obstructive pulmonary disease in a subject in need thereof, comprising administering a compound or active agent as described herein to the subject in an amount effective to treat chronic obstructive pulmonary disease.

A further aspect of the invention is a method of treating Alzheimer's disease in a subject in need thereof, comprising administering a compound or active agent as described herein to the subject in an amount effective to treat Alzheimer's disease.

A further aspect of the invention is a method of treating type II diabetes in a subject in need thereof, comprising administering a compound or active agent as described herein to the subject in an amount effective to treat type II diabetes.

A further aspect of the invention is a method of treating cancer in a subject in need thereof, comprising administering a compound or active agent as described herein to the subject in an amount effective to treat cancer.

A further aspect of the invention is a method of treating hypertension in a subject in need thereof, comprising administering a compound or active agent as described herein to the subject in an amount effective to treat hypertension.

A further aspect of the invention is a method of treating erectile dysfunction in a subject in need thereof, comprising administering a compound or active agent as described herein to the subject in an amount effective to treat erectile dysfunction.

A further aspect of the invention is the use of a compound or active agent as described herein for the preparation of a medicament for carrying out a method as described herein.

The present invention is explained in greater detail below.

### BRIEF DESCRIPTION OF THE DRAWINGS

FIGS. 1A-C show the effects of CCI-7155 (50 and 100 mg/kg/day p.o.), CCI-7156 (100 mg/kg/day p.o.) and sulfasalazine (50 mg/kg/day p.o.) on body weight, expressed a % change in body weight at Day 0.

FIG. 2 shows the effects of CCI-7155 (50 and 100 mg/kg/day p.o.), CCI-7156 (100 mg/kg/day p.o.) and sulfasalazine (50 mg/kg/day p.o.) on macroscopic injury in the colon.

FIG. 3 shows the effects of CCI-7155 (50 and 100 mg/kg/day p.o.), CCI-7156 (100 mg/kg/day p.o.) and sulfasalazine (50 mg/kg/day p.o.) on colon weight. Compounds were given in divided doses in a twice a day dosing schedule.

FIG. 4 shows the effects of CCI-7155 (50 and 100 mg/kg/day p.o.), CCI-7156 (100 mg/kg/day p.o.) and sulfasalazine (50 mg/kg/day p.o.) on water content in the colon. Compounds were given in divided doses in a twice a day dosing schedule.

FIG. 5. shows the effects of CCI-7155 (50 and 100 mg/kg/day given p.o. in divided doses, b.i.d.), CCI-7156 (100 mg/kg/day given p.o. in divided doses, b.i.d.) and sulfasalazine (50 mg/kg/day given p.o. in divided doses, b.i.d) on MPO levels in the colon, expressed as mU/mg protein.

FIG. 6 shows the effects of CCI-7308 (4, 20 and 100 mg/kg/day p.o.) or sulfasalazine (50 mg/kg/day p.o.) on body weight, expressed a % change in body weight at Day 0.

FIG. 7 show the effects of CCI-7308 (4, 20 and 100 mg/kg/day p.o) or sulfasalazine (50 mg/kg/day p.o.) on macroscopic injury in the colon.

FIG. 8 shows the effects of CCI-7308 (4, 20 and 100 mg/kg/day p.o) or sulfasalazine (50 mg/kg/day p.o.) on colon weight.

FIG. 9 shows the effects of CCI-7308 (4, 20 and 100 mg/kg/day p.o) or sulfasalazine (50 mg/kg/day p.o.) on TNF-α levels in the colon, expressed as pg/mg protein.

FIGS. 10A-10C show the effects of CCI-7506 (50 and 100 mg/kg/day p.o.), CCI-7507 (25 and 50 mg/kg/day p.o.), sulfasalazine (50 mg/kg/day p.o.) or infliximab (3 mg/kg i.v on Day 1 and 7) on body weight over 14 days, expressed a % change of the body weight at Day −1, prior to TNBS challenge on Day 0.

FIG. 11 shows the effects of CCI-7506 (50 and 100 mg/kg/day p.o.), CCI-7507 (25 and 50 mg/kg/day p.o.), sulfasalazine (SASP, 50 mg/kg/day p.o.) or infliximab (3 mg/kg i.v on Day 1 and 7) on macroscopic injury in the colon, determined 14 days after TNBS challenge, as assessed as the colonic lesion area, % of the total area measured.

FIG. 12. shows the effects of CCI-7506 (50 and 100 mg/kg/day p.o.), CCI-7507 (25 and 50 mg/kg/day p.o.), sulfasalazine (50 mg/kg/day p.o.) or infliximab (3 mg/kg i.v on Day 1 and 7) on macroscopic injury in the colon, determined 14 days after TNBS challenge, as assessed by a Damage Score (0-5 scale).

FIG. 13. shows the effects of CCI-7506 (50 and 100 mg/kg/day p.o.), CCI-7507 (25 and 50 mg/kg/day p.o.), sulfasalazine (50 mg/kg/day p.o.) or infliximab (3 mg/kg i.v on Day 1 and 7) on colon weight, determined 14 days after TNBS challenge.

FIG. 14 shows the effects of CCI-7506 (50 and 100 mg/kg/day p.o.), CCI-7507 (25 and 50 mg/kg/day p.o.), sulfasalazine (50 mg/kg/day p.o.) or infliximab (3 mg/kg i.v on Day 1 and 7) on MPO levels in the colon, expressed as mU/mg protein, determined 14 days after TNBS challenge.

FIG. 15 shows the effects of CCI-7506 (50 and 100 mg/kg/day p.o.), CCI-7507 (25 and 50 mg/kg/day p.o.), sulfasalazine (50 mg/kg/day p.o.) or infliximab (3 mg/kg i.v on Day 1 and 7) on TNF-α levels in the colon, expressed as pg/mg protein, determined 14 days after TNBS challenge.

FIG. 16 shows the effect of prophylactic topical treatment with a presently disclosed compound on arachidonic acid-induced murine ear edema.

### DETAILED DESCRIPTION

A variety of substituent groups are utilized herein, including hydrogen and the groups identified herein. In addition, R groups on adjacent carbons may be joined together to form ring structures, including cycloalkyl and aryl groups. “Halo” as used herein refers to any suitable halogen, including —F, —Cl, —Br, and —I.

“Mercapto” as used herein refers to an —SH group.

“Azido” as used herein refers to an —N₃ group.

“Cyano” as used herein refers to a —CN group.

“Hydroxyl” as used herein refers to an —OH group.

“Nitro” as used herein refers to an —NO₂ group.

“Oxy” as used herein refers to a —O— group.

“Oxo” as used herein refers to a ═O group.

“Alkyl” as used herein alone or as part of another group, refers to a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like. “Loweralkyl” as used herein, is a subset of alkyl, in some embodiments preferred, and refers to a straight or branched chain hydrocarbon group containing from 1 to 4 carbon atoms. Representative examples of lower alkyl include, but are not limited to, methyl, ethyl, n-propyl, iso-propyl, n-butyl, iso-butyl, tert-butyl, and the like. Alkyl and loweralkyl groups may be unsubstituted or substituted one or more times with R groups as defined herein including halo, alkyl, haloalkyl, alkenyl, alkynyl, cycloalkyl, cycloalkylalkyl, aryl, arylalkyl, heterocyclo, heterocycloalkyl, hydroxyl, alkoxy, alkenyloxy, alkynyloxy, haloalkoxy, cycloalkoxy, cycloalkylalkyloxy, aryloxy, arylalkyloxy, heterocyclooxy, heterocyclolalkyloxy, mercapto, alkyl-S(O)m, haloalkyl-S(O)m, alkenyl-S(O)m, alkynyl-S(O)m, cycloalkyl-S(O)m, cycloalkylalkyl-S(O)m, aryl-S(O)m, arylalkyl-S(O)m, heterocyclo-S(O)m, heterocycloalkyl-S(O)m, amino, alkylamino, alkenylamino, alkynylamino, haloalkylamino, cycloalkylamino, cycloalkylalkylamino, arylamino, arylalkylamino, heterocycloamino, heterocycloalkylamino, disubstituted-amino, acylamino, acyloxy, ester, amide, sulfonamide, urea, alkoxyacylamino, aminoacyloxy, nitro or cyano where m=0, 1 or 2.

“Alkenyl” as used herein alone or as part of another group, refers to a straight or branched chain hydrocarbon containing from 1 to 10 carbon atoms which include 1 to 4 double bonds in the normal chain. Representative examples of Alkenyl include, but are not limited to, vinyl, 2-propenyl, 3-butenyl, 2-butenyl, 4-pentyl, 3-pentyl, 2-hexenyl, 3-hexenyl, 2,4-heptadiene, and the like. These groups may be optionally substituted in like manner as described with alkyl above.

“Alkynyl” as used herein alone or as part of another group, refers to a straight or branched chain hydrocarbon containing from 1 to 10 carbon atoms which include 1 triple bond in the normal chain. Representative examples of Alkynyl include, but are not limited to, 2-propynyl, 3-butynyl, 2-butynyl, 4-pentenyl, 3-pentenyl, and the like. These groups may be optionally substituted in like manner as described with alkyl above.

“Alkoxy,” as used herein alone or as part of another group, refers to an alkyl group, as defined herein, appended to the parent molecular moiety through an oxy group, as defined herein. Representative examples of alkoxy include, but are not limited to, methoxy, ethoxy, propoxy, 2-propoxy, butoxy, tert-butoxy, pentyloxy, hexyloxy and the like. These groups may be optionally substituted in like manner as described with alkyl above.

“Acyl” as used herein alone or as part of another group, refers to a —C(O)R radical, where R is any suitable substituent such as alkyl, alkenyl, alkynyl, aryl, alkylaryl, etc. as given herein.

“Haloalkyl,” as used herein alone or as part of another group, refers to at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, 2-chloro-3-fluoropentyl, and the like.

“Alkylthio,” as used herein alone or as part of another group, refers to an alkyl group, as defined herein, appended to the parent molecular moiety through a thio moiety. Representative examples of alkylthio include, but are not limited, methylthio, ethylthio, tert-butylthio, hexylthio, and the like.

“Aryl,” as used herein alone or as part of another group, refers to a monocyclic carbocyclic ring system or a bicyclic carbocyclic fused ring system having one or more aromatic rings. Representative examples of aryl include, azulenyl, indanyl, indenyl, naphthyl, phenyl, tetrahydronaphthyl, and the like. These rings may be optionally substituted with groups selected from halo, alkyl, haloalkyl, alkenyl, alkynyl, cycloalkyl, cycloalkylalkyl, aryl, arylalkyl, heterocyclo, heterocycloalkyl, hydroxyl, alkoxy, alkenyloxy, alkynyloxy, haloalkoxy, cycloalkoxy, cycloalkylalkyloxy, aryloxy, arylalkyloxy, heterocyclooxy, heterocyclolalkyloxy, mercapto, alkyl-S(O)m, haloalkyl-S(O)m, alkenyl-S(O)m, alkynyl-S(O)m, cycloalkyl-S(O)m, cycloalkylalkyl-S(O)m, aryl-S(O)m, arylalkyl-S(O)m, heterocyclo-S(O)m, heterocycloalkyl-S(O)m, amino, alkylamino, alkenylamino, alkynylamino, haloalkylamino, cycloalkylamino, cycloalkylalkylamino, arylamino, arylalkylamino, heterocycloamino, heterocycloalkylamino, disubstituted-amino, acylamino, acyloxy, ester, amide, sulfonamide, urea, alkoxyacylamino, aminoacyloxy, nitro or cyano where m=0, 1 or 2.

“Arylalkyl,” as used herein alone or as part of another group, refers to an aryl group, as defined herein, appended to the parent molecular moiety through an alkyl group, as defined herein. Representative examples of arylalkyl include, but are not limited to, benzyl, 2-phenylethyl, 3-phenylpropyl, 2-naphth-2-ylethyl, and the like.

“Amino” as used herein means the radical —NH₂.

“Alkylamino” as used herein alone or as part of another group means the radical —NHR, where R is an alkyl group.

“Arylalkylamino” as used herein alone or as part of another group means the radical —NHR, where R is an arylalkyl group.

“Disubstituted-amino” as used herein alone or as part of another group means the radical —NRₐR_{b}, where Rₐ and R_{b} are independently selected from the groups alkyl, haloalkyl, alkenyl, alkynyl, cycloalkyl, cycloalkylalkyl, aryl, arylalkyl, heterocyclo, heterocycloalkyl.

“Acylamino” as used herein alone or as part of another group means the radical —NRₐR_{b}, where Rₐ is an acyl group as defined herein and R_{b} is selected from the hydrogen, alkyl, haloalkyl, alkenyl, alkynyl, cycloalkyl, cycloalkylalkyl, aryl, arylalkyl, heterocyclo, heterocycloalkyl.

“Acyloxy” as used herein alone or as part of another group means the radical —OR, where R is an acyl group as defined herein.

“Ester” as used herein alone or as part of another group refers to a —C(O)OR radical, where R is any suitable substituent such as alkyl, aryl, alkylaryl, etc.

“Amide” as used herein alone or as part of another group refers to a —C(O)NRₐR_{b} radical, where Rₐ and R_{b} are any suitable substituent such as alkyl, aryl, alkylaryl, etc.

“Sulfonamide” as used herein alone or as part of another group refers to a —S(O)₂NRₐR_{b} radical, where Rₐ and R_{b} are any suitable substituent, such as H, alkyl, aryl, alkylaryl, etc.

“Sulfone” as used herein alone or as part of another group refers to a —S(O)₂R radical, where R is any suitable substituent, such as H, alkyl, aryl, alkylaryl, etc.

“Aminosulfonyl” as used herein alone or as part of another group refers to a —N(Rₐ)S(O)₂R_{b} radical, where Rₐ and R_{b} are any suitable substituent, such as H, alkyl, aryl, alkylaryl, etc.

“Urea” as used herein alone or as part of another group refers to an —N(R_{c})C(O)NRₐR_{b} radical, where Rₐ, R_{b} and R_{c} are any suitable substituent such as H, alkyl, aryl, alkylaryl, etc.

“Alkoxyacylamino” as used herein alone or as part of another group refers to an —N(Rₐ)C(O)OR_{b} radical, where Rₐ, R_{b} are any suitable substituent such as H, alkyl, aryl, alkylaryl, etc.

“Aminoacyl” as used herein alone or as part of another group refers to an —C(O)NRₐR_{b} radical, where Rₐ and R_{b} are any suitable substituent, such as H, alkyl, aryl, alkylaryl, etc.

“Aminoacyloxy” as used herein alone or as part of another group refers to an —OC(O)NRₐR_{b} radical, where Rₐ and R_{b} are any suitable substituent, such as H, alkyl, aryl, alkylaryl, etc.

“Cycloalkyl,” as used herein alone or as part of another group, refers to a saturated or partially unsaturated cyclic hydrocarbon group containing from 3, 4 or 5 to 6, 7 or 8 carbons (which may be replaced in a heterocyclic group as discussed below). Representative examples of cycloalkyl include, cyclopropyl, cyclobutyl, cyclopentyl, cyclohexyl, cycloheptyl, and cyclooctyl. These rings may be optionally substituted with halo or loweralkyl.

“Heterocyclic group” or “heterocycle” as used herein alone or as part of another group, refers to a monocyclic- or a bicyclic-ring system. Monocyclic ring systems are exemplified by any 5 or 6 membered ring containing 1, 2, 3, or 4 heteroatoms independently selected from oxygen, nitrogen and sulfur. The 5 membered ring has from 0-2 double bonds and the 6 membered ring has from 0-3 double bonds. Representative examples of monocyclic ring systems include, but are not limited to, azetidine, azepine, aziridine, diazepine, 1,3-dioxolane, dioxane, dithiane, furan, imidazole, imidazoline, imidazolidine, isothiazole, isothiazoline, isothiazolidine, isoxazole, isoxazoline, isoxazolidine, morpholine, oxadiazole, oxadiazoline, oxadiazolidine, oxazole, oxazoline, oxazolidine, piperazine, piperidine, pyran, pyrazine, pyrazole, pyrazoline, pyrazolidine, pyridine, pyrimidine, pyridazine, pyrrole, pyrroline, pyrrolidine, tetrahydrofuran, tetrahydrothiophene, tetrazine, tetrazole, thiadiazole, thiadiazoline, thiadiazolidine, thiazole, thiazoline, thiazolidine, thiophene, thiomorpholine, thiomorpholine sulfone, thiopyran, triazine, triazole, trithiane, and the like. Bicyclic ring systems are exemplified by any of the above monocyclic ring systems fused to an aryl group as defined herein, a cycloalkyl group as defined herein, or another monocyclic ring system as defined herein. Representative examples of bicyclic ring systems include but are not limited to, for example, benzimidazole, benzothiazole, benzothiadiazole, benzothiophene, benzoxadiazole, benzoxazole, benzofuran, benzopyran, benzothiopyran, benzodioxine, 1,3-benzodioxole, cinnoline, indazole, indole, indoline, indolizine, naphthyridine, isobenzofuran, isobenzothiophene, isoindole, isoindoline, isoquinoline, phthalazine, purine, pyranopyridine, quinoline, quinolizine, quinoxaline, quinazoline, tetrahydroisoquinoline, tetrahydroquinoline, thiopyranopyridine, and the like. These rings may be optionally substituted with groups selected from halo, alkyl, haloalkyl, alkenyl, alkynyl, cycloalkyl, cycloalkylalkyl, aryl, arylalkyl, heterocyclo, heterocycloalkyl, hydroxyl, alkoxy, alkenyloxy, alkynyloxy, haloalkoxy, cycloalkoxy, cycloalkylalkyloxy, aryloxy, arylalkyloxy, heterocyclooxy, heterocyclolalkyloxy, mercapto, alkyl-S(O)m, haloalkyl-S(O)m, alkenyl-S(O)m, alkynyl-S(O)m, cycloalkyl-S(O)m, cycloalkylalkyl-S(O)m, aryl-S(O)m, arylalkyl-S(O)m, heterocyclo-S(O)m, heterocycloalkyl-S(O)m, amino, alkylamino, alkenylamino, alkynylamino, haloalkylamino, cycloalkylamino, cycloalkylalkylamino, arylamino, arylalkylamino, heterocycloamino, heterocycloalkylamino, disubstituted-amino, acylamino, acyloxy, ester, amide, sulfonamide, urea, alkoxyacylamino, aminoacyloxy, nitro or cyano where m=0, 1 or 2.

“Oxoheterocyclic group” refers to a heterocyclic group such as described above, substituted with one or more oxo groups, such as pyridine-N-oxide.

“Arylthio” as used herein refers to a group of the formula —S—R, where R is aryl as described above.

“Hydroxyamino” as used herein refers to a group of the formula —N(R)OH, where R is any suitable group such as alkyl, aryl, alkylaryl, etc.

“Treat” as used herein refers to any type of treatment that imparts a benefit to a patient afflicted with a disease, including improvement in the condition of the patient (e.g., in one or more symptoms), delay in the progression of the disease, etc.

“Inflammatory bowel disease” as used herein includes both Crohn's disease and ulcerative colitis.

“Cancer” as used herein includes any cancer, particularly solid tumors, and includes but is not limited to lung cancer, colon cancer, breast cancer, prostate cancer, liver cancer, skin cancer, ovarian cancer, etc.

“Pharmaceutically acceptable” as used herein means that the compound or composition is suitable for administration to a subject to achieve the treatments described herein, without unduly deleterious side effects in light of the severity of the disease and necessity of the treatment.

“Pharmaceutically acceptable prodrugs” as used herein refers to those prodrugs of the compounds of the present invention which are, within the scope of sound medical judgment, suitable for use in contact with the tissues of humans and lower animals without undue toxicity, irritation, allergic response and the like, commensurate with a reasonable risk/benefit ratio, and effective for their intended use, as well as the zwitterionic forms, where possible, of the compounds of the invention. The term “prodrug” refers to compounds that are rapidly transformed in vivo to yield the parent compound of the above formulae, for example, by hydrolysis in blood. A thorough discussion is provided in T. Higuchi and V. Stella, Prodrugs as Novel delivery Systems,

Vol. 14 of the A.C.S. Symposium Series and in Edward B. Roche, ed., Bioreversible Carriers in Drug Design, American Pharmaceutical Association and Pergamon Press, 1987, both of which are incorporated by reference herein. See also U.S. Pat. No. 6,680,299 Examples include a prodrug that is metabolized in vivo by a subject to an active drug having an activity of active compounds as described herein, wherein the prodrug is an ester of an alcohol or carboxylic acid group, if such a group is present in the compound; an acetal or ketal of an alcohol group, if such a group is present in the compound; an N-Mannich base or an imine of an amine group, if such a group is present in the compound; or a Schiff base, oxime, acetal, enol ester, oxazolidine, or thiazolidine of a carbonyl group, if such a group is present in the compound, such as described in U.S. Pat. No. 6,680,324 and U.S. Pat. No. 6,680,322.

Prodrugs of the present invention include esters or compositions as described in U.S. Pat. No. 6,548,668 to Adams et al., U.S. Pat. No. 6,083,903 to Adams et al., or U.S. Pat. No. 6,699,835 to Plamondon et al., the disclosures of which are incorporated by reference herein in their entirety.

### 1. Active Compounds.

Active compounds of the present invention (this term including pharmaceutically acceptable salts and prodrugs thereof) can be made in accordance with known techniques (see, e.g., U.S. Pat. No. 5,643,893 to Benson et al.) or variations thereof which will be apparent to those skilled in the art based on the disclosure provided herein. In some embodiments, active compounds of the present disclosure are compounds of Formula I or Formula II:

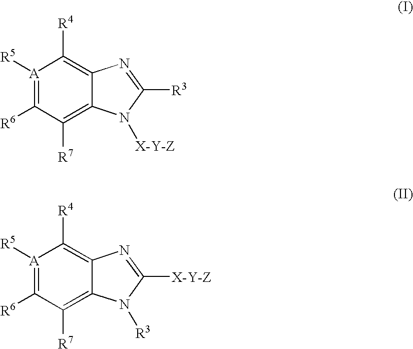

wherein:

A is N or C, subject to the proviso that R⁵ is absent when A is N;

X is, for Formula I, —C(O)—, —S(O)₂—, or a covalent bond, more preferably —S(O)₂—, or a covalent bond, and X is, for Formula II, —C(O)—, —S(O)₂—, or a covalent bond;

Y is a linking group such as alkyl (e.g., —R— where R is C2-C6 alkyl), alkenyl (e.g., —R— where R is C2-C6 alkenyl), cycloalkyl (e.g., —R— where R is C3-C6 cycloalkyl), alkylcycloalkyl(e.g., —R—R′—, where R is C1-C4 alkyl and R′ is C3-C6 cycloalkyl), cylcoalkylalkyl (e.g., —R—R′—, where R is C3-C6 cycloalkyl and R′ is C1-C4 alkyl), alkylcycloalkylalkyl (e.g., —R—R′—R″—, wherein R is C1-C4 alkyl, R′ is C3-C6 cycloalkyl, and R″ is C1-C4 alkyl), alkyloxyalkyl (e.g., —R—O—R′—, wherein R and R′ are C1-C4 alkyl); aryl (e.g., —R— where R is aryl), alkylaryl (e.g., —R—R′— where R is C1-C4 alkyl and R′ is aryl), alkylarylalkyl (e.g., —R—R′—R″— where R is C1-C4 alkyl, R′ is aryl, and R″ is C1-C4 alkyl), or arylalkyl (e.g., —R—R′— where R is aryl alkyl and R′ is C1-C4 alkyl); cycloalkylalkyl (e.g. —R—R′—, where R is C3-C6 cycloalkyl and R′ is C1-C4 alkyl), alkylheterocycle (e.g., —R—R′, where R is C1-C4 alkyl and R′ is a heterocyclic group as described herein), heterocycloalkyl, alkylheterocycloalkyl, heterocycle, aminoalkyl (e.g., —N(R)R′—, where R is H or C 1-C4 alkyl and R′ is C 1-C4 alkyl), oxyalkyl (e.g., —O—R— where R is C2-C6 alkyl), aminoaryl (e.g., —N(R)R′—, where R is H or C1-C4 alkyl and R′ is aryl), and oxyaryl (e.g., —O—R—, where R is aryl); and

Z is selected from the group consisting of —B(OR¹)OR², —CON(R¹)OR², and —N(OR¹)COR² or any of the additional alternatives for Z described in greater detail below.

R¹ and R² are each independently H, loweralkyl, or together form C₂-C₄ alkylene; and

R³, R⁴, R⁵, R⁶, and R⁷ are each independently selected from the group consisting of: H, halo, loweralkyl, haloloweralkyl, haloloweralkoxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, nitro; arylalkyloxy, cycloalkyloxy, cycloalkylalkoxy, cycloalkylamino, urea, cycloalkylalkylamino, cycloalkyl, alkylcycloalkyl, hydroxyamino, alkoxyacylamino, and arylthio; and 5- or 6-membered organic rings containing 0 to 4 heteroatoms selected from the group consisting of N, O and S, which rings may be unsubstituted or substituted from 1 to 4 times with halo, loweralkyl, haloloweralkyl, haloloweralkyloxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, nitro; and oxoheterocyclic groups; or a pharmaceutically acceptable salt or prodrug thereof.

In some embodiments, R³ is preferably not H. Thus in some embodiments R³ is preferably a 5- or 6-membered organic ring containing 0 to 4 heteroatoms selected from the group consisting of N, O and S, which ring may be unsubstituted or substituted from 1 to 4 times with halo, cycloalkylalkoxy, loweralkyl, haloloweralkyl, haloloweralkyloxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, nitro; and oxoheterocyclic groups.

It will be understood that, in Formula II where R³ is bonded to the ring nitrogen, it is less preferred for R³ to be halo, azido, mercapto, amino, alkylamino, dialkylamino, acylamino, cyano, and arylalkylamino, and more preferred for R³ to be alkyl, loweralkyl, and haloloweralkyl, sulfone, amide, and. aryl.

R⁵ is preferably selected from the group consisting of: halo, loweralkyl, haloloweralkyl, haloloweralkyloxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, and nitro. R⁵ is more preferably selected from the group consisting of: halo, haloloweralkyl, haloloweralkyloxy, loweralkoxy, amino, acylamino, aminoacyl, arylalkyl, aryloxy, acyl, arylamino, cyano, nitro, and heterocycleamino. R⁵ is most preferably cyano, fluoroalkyl or halo.

R⁴ is in some embodiments preferably H. In other embodiments R⁴ is preferably selected from the group consisting of: halo, loweralkyl, haloloweralkyl, haloloweralkyloxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, and nitro; more preferably R⁴ is selected from the group consisting of: halo, haloloweralkyl, haloloweralkyloxy, loweralkoxy, amino, acylamino, aminoacyl, arylalkyl, aryloxy, acyl, arylamino, cyano, nitro, and heterocycleamino, and still more preferably R⁴ is cyano, fluoroalkyl or halo.

In some embodiments R⁶ is H. In other embodiments R⁶ is preferably selected from the group consisting of: halo, loweralkyl, haloloweralkyl, haloloweralkyloxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, and nitro, in such other embodiments R⁶ is more preferably selected from the group consisting of: halo, haloloweralkyl, haloloweralkyloxy, loweralkoxy, amino, acylamino, aminoacyl, arylalkyl, aryloxy, acyl, arylamino, cyano, nitro, and heterocycleamino; in such other embodiments R⁶ is most preferably cyano, fluoroalkyl or halo.

In some embodiments, at least two of R⁴, R⁶, and R⁷ are H. In some preferred embodiments R⁶ and R⁷ are both H. In some preferred embodiments R⁷ is H.

Particularly preferred examples of compounds of the present invention are:
4-(2-(Trifluoromethyl)-1H-benzo[d]imidazol-1-yl)butylboronic acid;
5-(2-(Thiazol-4-yl)-1H-benzo[d]imidazol-1-yl)pentylboronic acid;
5-(5,6-dimethyl-1H-benzo[d]imidazol-1-yl)pentylboronic acid;
5-(1H-imidazo[4,5-c]pyridin-1-yl)pentylboronic acid;
5-(2-(4-Methoxyphenyl)-1H-benzo[d]imidazol-1-yl)pentylboronic acid;
5-(2-(3-Fluoro-4-methoxyphenyl)-1H-benzo[d]imidazol-1-yl)pentylboronic acid;
5-(5-cyano-2-(4-methoxyphenyl)-1H-benzo[d]imidazol-1-yl)pentylboronic acid;
5-(6-cyano-2-(4-methoxyphenyl)-1H-benzo[d]imidazol-1-yl)pentylboronic acid;

and pharmaceutically acceptable salts and prodrugs thereof.

Another aspect of the present disclosure are compounds of Formula III, Formula IV or Formula V:

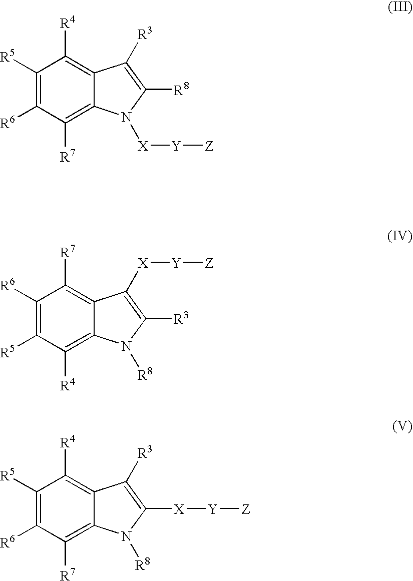

wherein:

X is, for Formula III, —C(O)—, —S(O)₂—, or a covalent bond, more preferably —S(O)₂—, or a covalent bond, and X is, for Formulas IV and V, —C(O)—, —S(O)₂—, or a covalent bond;

Y is a linking group such as alkyl (e.g., —R— where R is C2-C6 alkyl), alkenyl (e.g., —R— where R is C2-C6 alkenyl), cycloalkyl (e.g., —R— where R is C3-C6 cycloalkyl), alkylcycloalkyl(e.g., —R—R′—, where R is C1-C4 alkyl and R′ is C3-C6 cycloalkyl), cylcoalkylalkyl (e.g., —R—R′—, where R is C3-C6 cycloalkyl and R′ is C1-C4 alkyl), alkylcycloalkylalkyl (e.g., —R—R′—R″—, wherein R is C1-C4 alkyl, R′ is C3-C6 cycloalkyl, and R″ is C1-C4 alkyl), alkyloxyalkyl (e.g., —R—O—R′—, wherein R and R′ are C1-C4 alkyl); aryl (e.g., —R— where R is aryl), alkylaryl (e.g., —R—R′— where R is C1-C4 alkyl and R′ is aryl), alkylarylalkyl (e.g., —R—R′—R″— where R is C1-C4 alkyl, R′ is aryl, and R″ is C1-C4 alkyl), arylalkyl (e.g., —R—R′— where R is aryl alkyl and R′ is C1-C4 alkyl), cycloalkylalkyl (e.g. —R—R′—, where R is C3-C6 cycloalkyl and R′ is C1-C4 alkyl), alkylheterocycle (e.g., —R—R′, where R is C1-C4 alkyl and R′ is a heterocyclic group as described herein), heterocycloalkyl, alkylheterocycloalkyl, heterocycle, aminoalkyl (e.g., —N(R)R′—, where R is H or C1-C4 alkyl and R′ is C1-C4 alkyl), oxyalkyl (e.g., —O—R— where R is C2-C6 alkyl), aminoaryl (e.g., —N(R)R′—, where R is H or C1-C4 alkyl and R′ is aryl), or oxyaryl (e.g., —O—R—, where R is aryl); and

Z is selected from the group consisting of —B(OR¹)OR², —CON(R¹)OR², and —N(OR¹)COR² or any of the additional alternatives for Z described in greater detail below.

R¹ and R² are each independently H, loweralkyl, or together form C₂-C₄ alkylene; and

R³, R⁴, R⁵, R⁶, and R⁷ are each independently selected from the group consisting of: H, halo, loweralkyl, haloloweralkyl, haloloweralkoxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, nitro arylalkyloxy, cycloalkyloxy, cycloalkylalkoxy, cycloalkylamino, urea, cycloalkylalkylamino, cycloalkyl, alkylcycloalkyl, hydroxyamino, alkoxyacylamino, and arylthio; and 5- or 6-membered organic rings containing 0 to 4 heteroatoms selected from the group consisting of N, O and S, which rings may be unsubstituted or substituted from 1 to 4 times with halo, loweralkyl, haloloweralkyl, haloloweralkyloxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, nitro; and oxoheterocyclic groups; or a pharmaceutically acceptable salt or prodrug thereof.

In some embodiments of compounds of Formulas III, IV and V, R⁵ is selected from the group consisting of: halo, loweralkyl, haloloweralkyl, haloloweralkyloxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, and nitro, more preferably R⁵ is selected from the group consisting of: halo, haloloweralkyl, haloloweralkyloxy, loweralkoxy, amino, acylamino, aminoacyl, arylalkyl, aryloxy, acyl, arylamino, cyano, nitro, and heterocycleamino, and most preferably R⁵ is cyano, fluoroalkyl or halo.

In some embodiments of compounds of Formulas III, IV and V, R⁴ is H; in other embodiments R⁴ is selected from the group consisting of: halo, loweralkyl, haloloweralkyl, haloloweralkyloxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, and nitro; more preferably from the group consisting of: halo, haloloweralkyl, haloloweralkyloxy, loweralkoxy, amino, acylamino, aminoacyl, arylalkyl, aryloxy, acyl, arylamino, cyano, nitro, and heterocycleamino; and most preferably cyano, fluoroalkyl or halo.

In some embodiments of compounds of Formulas III, IV and V, R⁶ is H; in other embodiments R⁶ is selected from the group consisting of: halo, loweralkyl, haloloweralkyl, haloloweralkyloxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, and nitro; more preferably halo, haloloweralkyl, haloloweralkyloxy, loweralkoxy, amino, acylamino, aminoacyl, arylalkyl, aryloxy, acyl, arylamino, cyano, nitro, and heterocycleamino; and most preferably cyano, fluoroalkyl or halo.

In some embodiments of compounds of Formulas III, IV and V, R⁷ is H; in other embodiments R⁷ is selected from the group consisting of: halo, loweralkyl, haloloweralkyl, haloloweralkyloxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, and nitro; more preferably halo, haloloweralkyl, haloloweralkyloxy, loweralkoxy, amino, acylamino, aminoacyl, arylalkyl, aryloxy, acyl, arylamino, cyano, nitro, and heterocycleamino; and most preferably cyano, fluoroalkyl or halo.

In some embodiments of compounds of Formulas III, IV and V, at least two of R⁴, R⁶, and R⁷ are H. For example, in some embodiments R⁶ and R⁷ are H; in other embodiments R⁴ and R⁶ are H; in other embodiments R⁵ and R⁷ are H; in still other embodiments R⁴ and R⁵ are H.

In yet another aspect of the present disclosure are compounds of Formula VI:

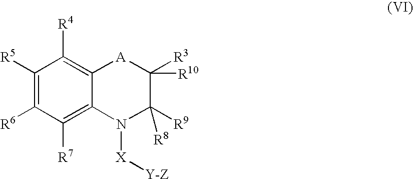

wherein:

A is S, O, SO₂ or NR;

X is —C(O)—, —S(O)₂—, or a covalent bond;

Y is a linking group such as alkyl (e.g., —R— where R is C2-C6 alkyl), alkenyl (e.g., —R— where R is C2-C6 alkenyl), cycloalkyl (e.g., —R— where R is C3-C6 cycloalkyl), alkylcycloalkyl(e.g., —R—R′—, where R is C1-C4 alkyl and R′ is C3-C6 cycloalkyl), cylcoalkylalkyl (e.g., —R—R′—, where R is C3-C6 cycloalkyl and R′ is C1-C4 alkyl), alkylcycloalkylalkyl (e.g., —R—R′—R″—, wherein R is C1-C4 alkyl, R′ is C3-C6 cycloalkyl, and R″ is C1-C4 alkyl), alkyloxyalkyl (e.g., —R—O—R′—, wherein R and R′ are C1-C4 alkyl); aryl (e.g., —R— where R is aryl), alkylaryl (e.g., —R—R′— where R is C1-C4 alkyl and R′ is aryl), alkylarylalkyl (e.g., —R—R′—R″— where R is C1-C4 alkyl, R′ is aryl, and R″ is C1-C4 alkyl), arylalkyl (e.g., —R—R′— where R is aryl alkyl and R′ is C1-C4 alkyl); cycloalkylalkyl (e.g. —R—R′— where R is C3-C6 cycloalkyl and R′ is C1-C4 alkyl), alkylheterocycle (e.g., —R—R′, where R is C1-C4 alkyl and R′ is a heterocyclic group as described herein), heterocycloalkyl, alkylheterocycloalkyl, heterocycle, aminoalkyl (e.g., —N(R)R′—, where R is H or C 1-C4 alkyl and R′ is C1-C4 alkyl), oxyalkyl (e.g., —O—R— where R is C2-C6 alkyl), aminoaryl (e.g., —N(R)R′—, where R is H or C1-C4 alkyl and R′ is aryl), and oxyaryl (e.g., —O—R—, where R is aryl); and

Z is selected from the group consisting of —B(OR¹)OR², —CON(R¹)OR², —N(OR¹)COR², or any of the additional alternatives for Z described in greater detail below.

In some embodiments of Formula VI, at least one of R³, R⁴, R⁵, R⁶, R⁷ or R⁸ is not H.

In some embodiments of Formula V¹, R⁵ is selected from the group consisting of: halo, loweralkyl, haloloweralkyl, haloloweralkyloxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, nitro, and hydroxyamino. In more preferred embodiments, R⁵ is selected from the group consisting of: halo, haloloweralkyl, haloloweralkyloxy, loweralkoxy, amino, acylamino, aminoacyl, arylalkyl, aryloxy, acyl, arylamino, cyano, nitro, and heterocycleamino. In still more preferred embodiments, R⁵ is cyano, fluoroalkyl or halo.

In some embodiments of Formula VI, R⁴ is H. In other embodiments of Formula VI, R⁴ is selected from the group consisting of: halo, loweralkyl, haloloweralkyl, haloloweralkyloxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, nitro and heterocycleamino; more preferably R⁴ is selected from the group consisting of: halo, haloloweralkyl, haloloweralkyloxy, loweralkoxy, amino, acylamino, aminoacyl, arylalkyl, aryloxy, acyl, arylamino, cyano, nitro, and heterocycleamino; and most preferably R⁴ is cyano, fluoroalkyl or halo.

In some embodiments of Formula VI, R⁶ is H. In other embodiments R⁶ is selected from the group consisting of: halo, loweralkyl, haloloweralkyl, haloloweralkyloxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, and nitro; more preferably halo, haloloweralkyl, haloloweralkyloxy, loweralkoxy, amino, acylamino, aminoacyl, arylalkyl, aryloxy, acyl, arylamino, cyano, nitro, and heterocycleamino; and most preferably R⁶ is cyano, fluoroalkyl or halo.

In some embodiments of Formula VI, R⁷ is H. In some preferred embodiments at least two of R⁴, R⁶, and R⁷ are H. In some still more preferred embodiments, R⁶ and R⁷ are H.

In some embodiments R is selected from the group consisting of H, loweralkyl, haloloweralkyl, haloloweralkyloxy, loweralkoxy, loweralkoxycarbo, carboxylic acid, acyl, acylamino, aminoacyl, arylalkyl, cyano, sulfonamide, aminosulfonyl, and sulfone; more preferably H, loweralkyl, haloloweralkyl, haloloweralkyloxy, loweralkoxy, loweralkoxycarbo, and arylalkyl.

In some embodiments R³ is selected from the group consisting of H, alkyl, aryl, heteroaryl, and cycloalkyl.

In some embodiments R⁸ and R⁹ are each independently selected from the group consisting of H and loweralkyl, or R⁸ and R⁹ are together ═O or ═S.

In some embodiments R⁹ and R¹⁰ are both H.

Examples of particularly preferred compounds of Formula VI include but are not limited to:

5-(6-fluoro-2,3-dihydro-3-oxobenzo[b][1,4]oxazin-4-yl)pentylboronic acid; 5-(2,3-dihydro-3-oxobenzo[b][1,4]thiazin-4-yl)pentylboronic acid; 5-(7-chloro-2,3-dihydro-3-oxobenzo[b][1,4]thiazin-4-yl)pentylboronic acid; 5-(2,3-dihydro-7-nitro-3-oxobenzo[b][1,4]oxazin-4-yl)pentylboronic acid; 5-(2,3-dihydro-3-oxobenzo[b][1,4]oxazin-4-yl)pentylboronic acid; ethyl 2-(3,4-dihydro-3-oxo-4-(5-pentylboronic acid)-2H-benzo[b][1,4]thiazin-2-yl)acetate; and pharmaceutically acceptable salts and prodrugs thereof.

In some embodiments, active compounds of the present disclosure are compounds of Formula VII:

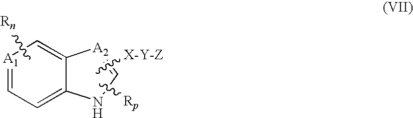

wherein:

A₁ and A₂ are each independently N or C

X is —C(O)—, —S(O)₂—, or a covalent bond,

Y is a linking group such as alkyl (e.g., —R— where R is C2-C6 alkyl), alkenyl (e.g., —R— where R is C2-C6 alkenyl), cycloalkyl (e.g., —R— where R is C3-C6 cycloalkyl), alkylcycloalkyl(e.g., —R—R′—, where R is C1-C4 alkyl and R′ is C3-C6 cycloalkyl), cylcoalkylalkyl (e.g., —R—R′—, where R is C3-C6 cycloalkyl and R′ is C1-C4 alkyl), alkylcycloalkylalkyl (e.g., —R—R′—R″—, wherein R is C1-C4 alkyl, R′ is C3-C6 cycloalkyl, and R″ is C1-C4 alkyl), alkyloxyalkyl (e.g., —R—O—R′—, wherein R and R′ are C1-C4 alkyl); aryl (e.g., —R— where R is aryl), alkylaryl (e.g., —R—R′— where R is C1-C4 alkyl and R′ is aryl), alkylarylalkyl (e.g., —R—R′—R″— where R is C1-C4 alkyl, R′ is aryl, and R″ is C1-C4 alkyl), or arylalkyl (e.g., —R—R′— where R is aryl alkyl and R′ is C1-C4 alkyl); cycloalkylalkyl (e.g. —R—R′—, where R is C3-C6 cycloalkyl and R′ is C1-C4 alkyl), alkylheterocycle (e.g., —R—R′, where R is C1-C4 alkyl and R′ is a heterocyclic group as described herein), heterocycloalkyl, alkylheterocycloalkyl, heterocycle, aminoalkyl (e.g., —N(R)R′—, where R is H or C1-C4 alkyl and R′ is C1-C4 alkyl), oxyalkyl (e.g., —O—R— where R is C2-C6 alkyl), aminoaryl (e.g., —N(R)R′—, where R is H or C1-C4 alkyl and R′ is aryl), and oxyaryl (e.g., —O—R—, where R is aryl); and

Z is selected from the group consisting of —B(OR¹)OR², —CON(R¹)OR², and —N(OR¹)COR² or any of the additional alternatives for Z described in greater detail below.

R¹ and R² are each independently H, loweralkyl, or together form C₂-C₄ alkylene; and

Rₙ, and Rₚ are each independently selected from the group consisting of: H, halo, loweralkyl, haloloweralkyl, haloloweralkoxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, nitro; arylalkyloxy, cycloalkyloxy, cycloalkylalkoxy, cycloalkylamino, urea, cycloalkylalkylamino, cycloalkyl, alkylcycloalkyl, hydroxyamino, alkoxyacylamino, and arylthio; and 5- or 6-membered organic rings containing 0 to 4 heteroatoms selected from the group consisting of N, O and S, which rings may be unsubstituted or substituted from 1 to 4 times with halo, loweralkyl, haloloweralkyl, haloloweralkyloxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, nitro; and oxoheterocyclic groups; subject to the proviso that when A₁ is C, then n=1 to 4; when A₁ is N, then n=1 to 3; A₂ is C, then p=1 to 2; when A₂ is N, then n=1; or a pharmaceutically acceptable salt or prodrug thereof.

In addition, compounds of the present invention include compounds of Formulas I, II, III, IV, V, VI, and VII, and others above in which substituent -Z is a group of the formula:

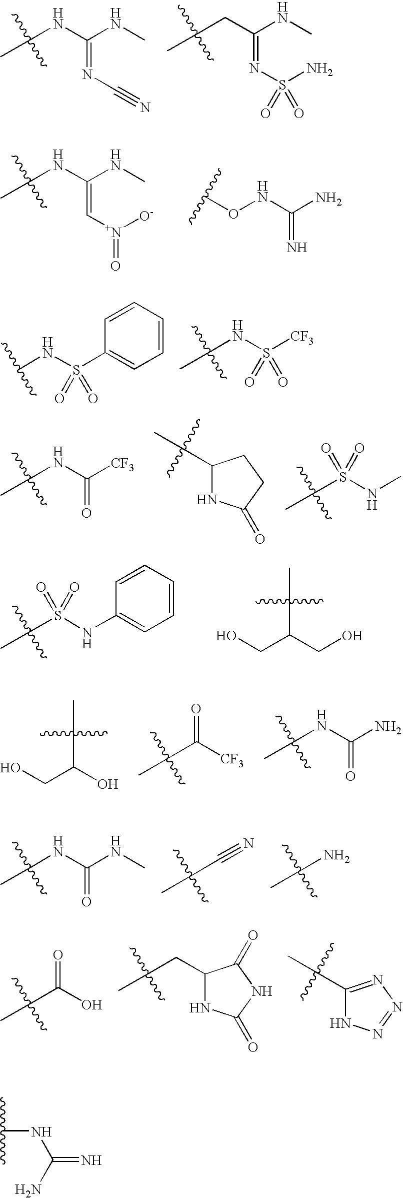

In addition, compounds of the present invention include compounds of Formulas I, II, III, IV, V, VI, and VII, and others herein substituent —Y-Z is a group of the formula:

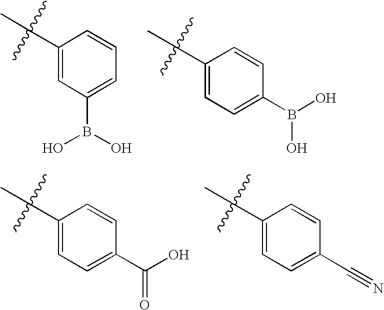

In addition, compounds of the invention include compounds of Formulas I, II, III, IV, V, VI, and VII, and others herein the groups —X—Y-Z are a substituent of the formula:

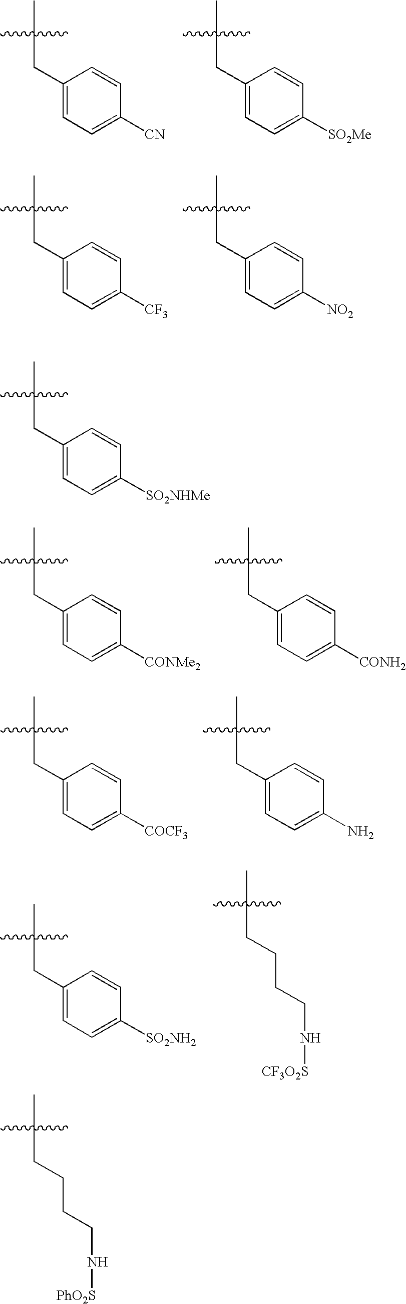

In addition, compounds of the invention include compounds of Formulas I, II, III, IV, V, VI, and VII, and others herein, the groups —X—Y-Z represent a substituent of the formula:

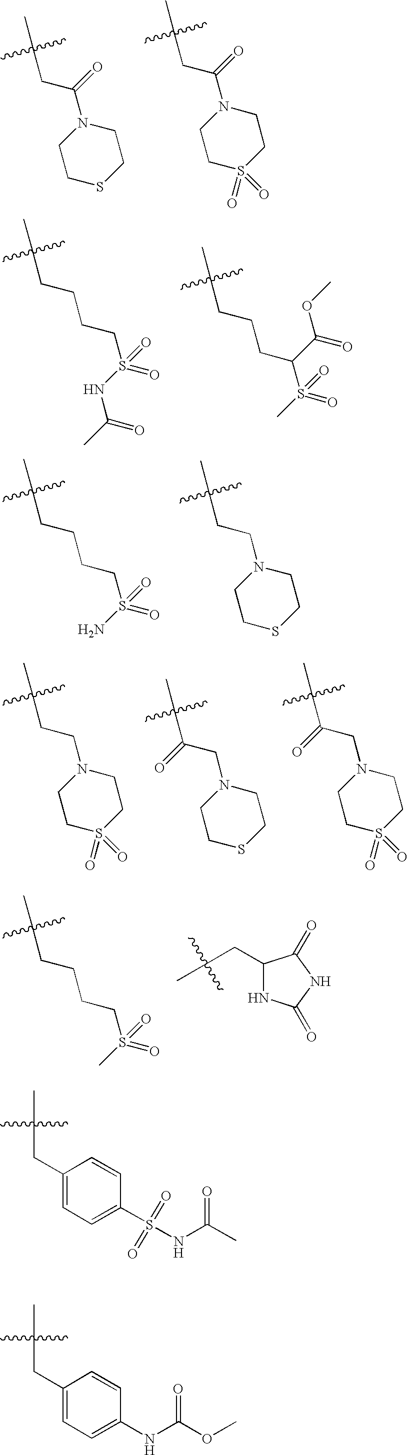

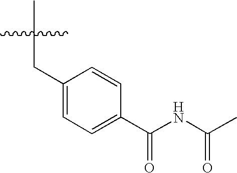

In addition, compounds of the invention include compounds of Formulas I, II, III, IV, V, VI, and VII, and others herein, group -Z is a substituent of the formula:

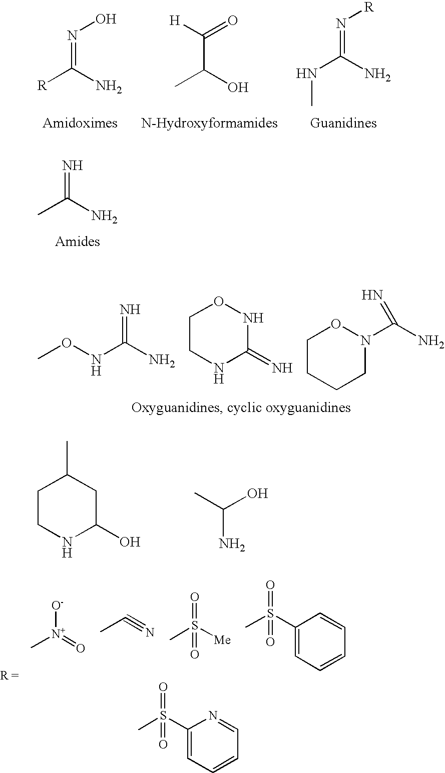

In addition, compounds of the invention includes compounds of the Formulas I, II, III, IV, V, VI, and VII, and others herein, group -Z is a substituent of the formula:

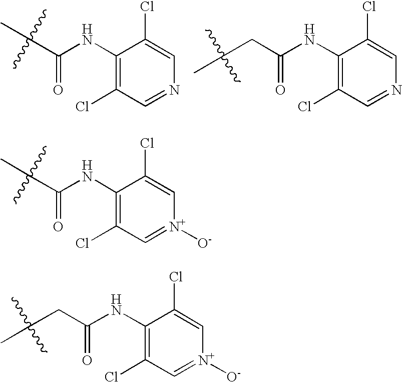

Examples of active compounds of the present invention include but are not limited to:

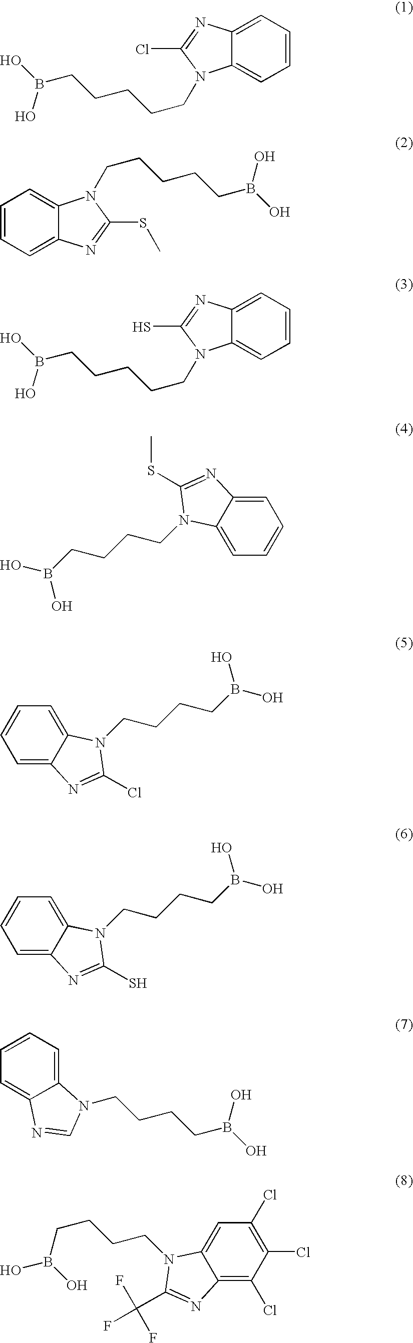

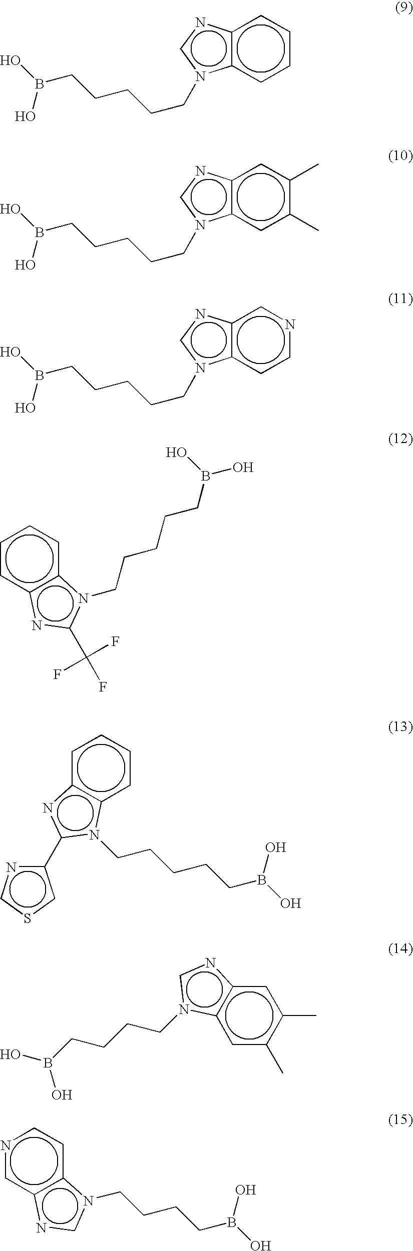

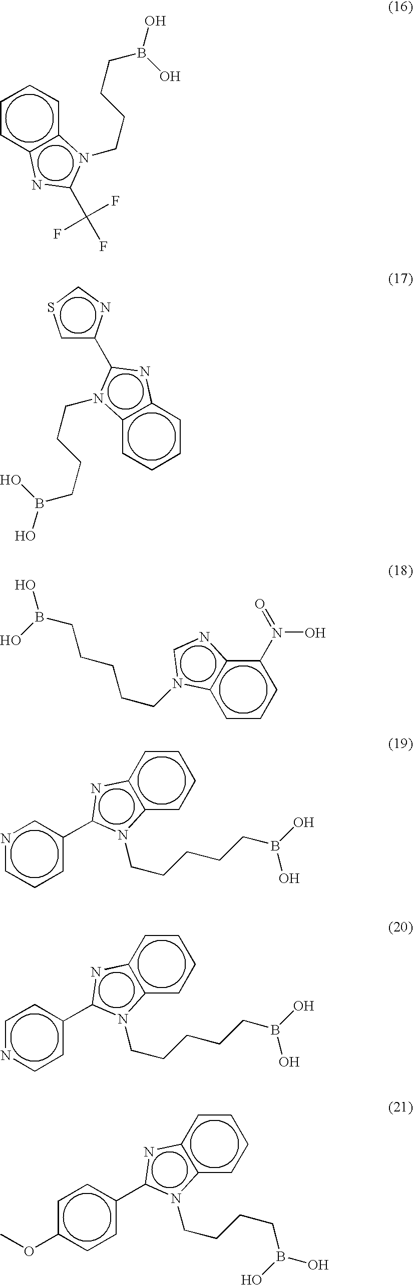

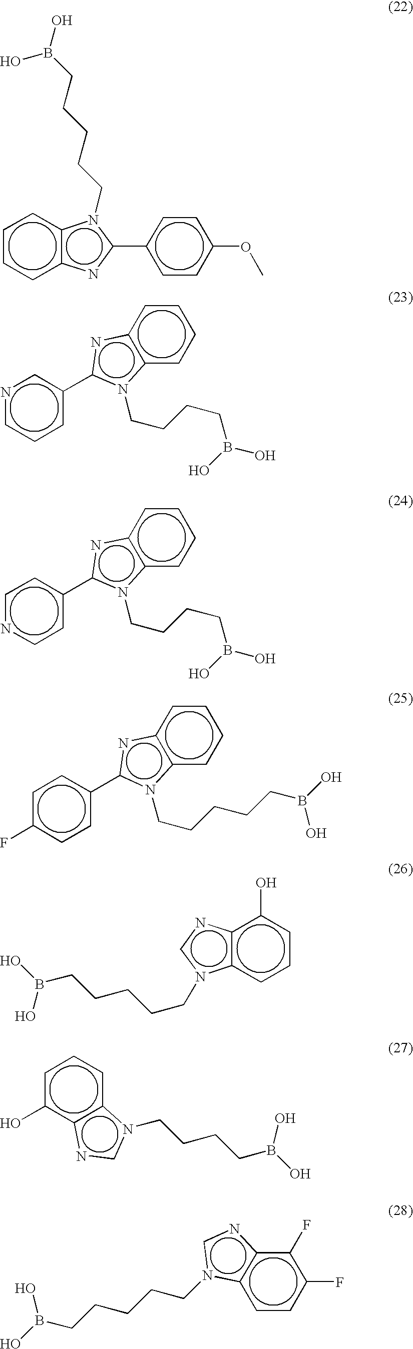

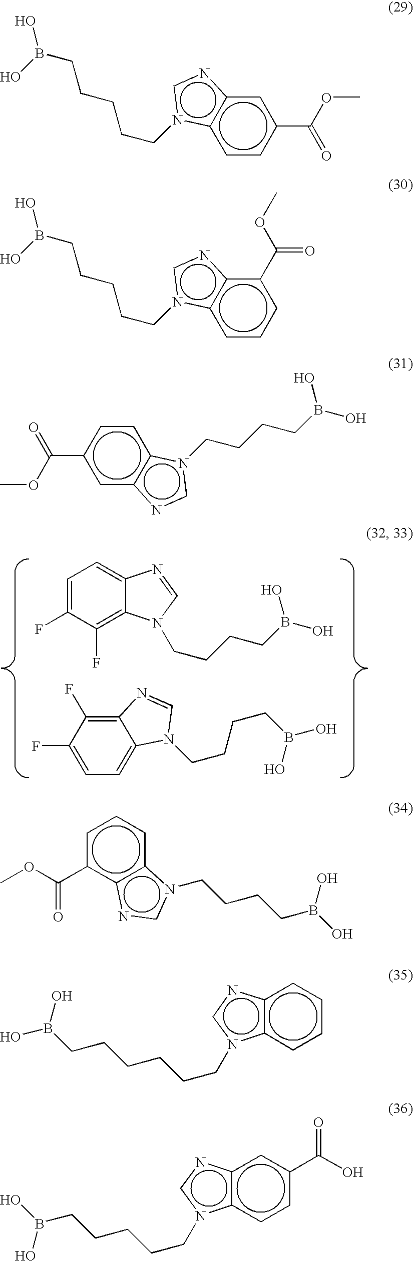

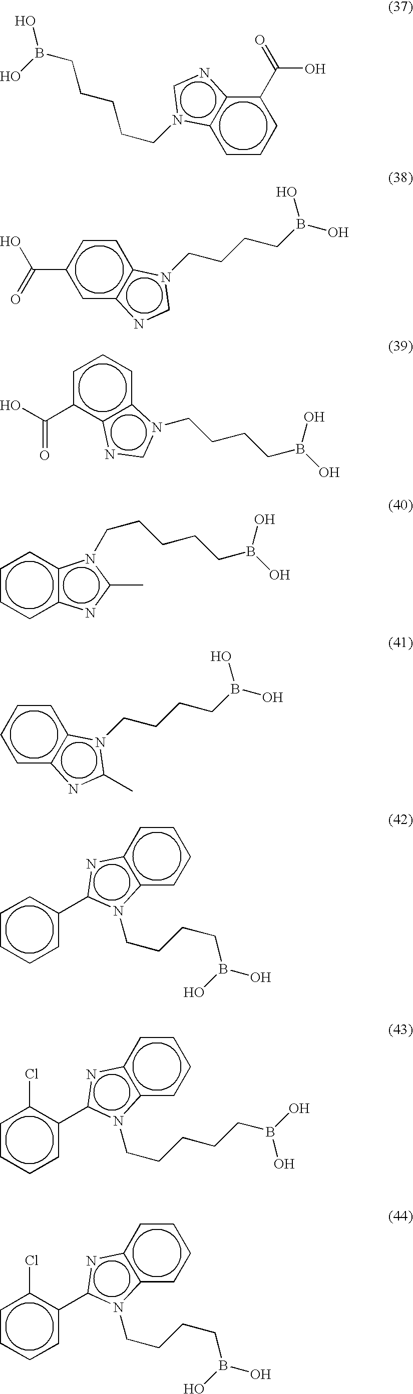

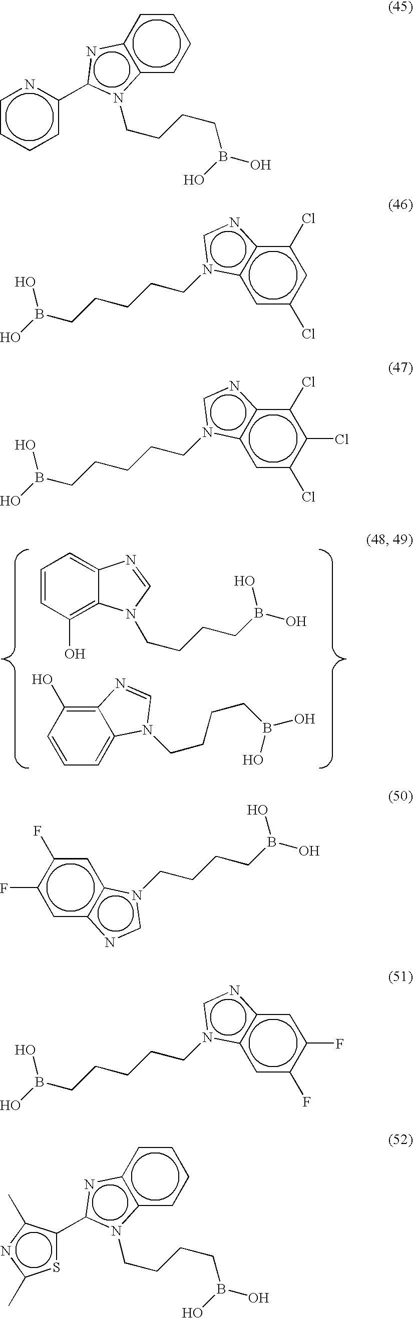

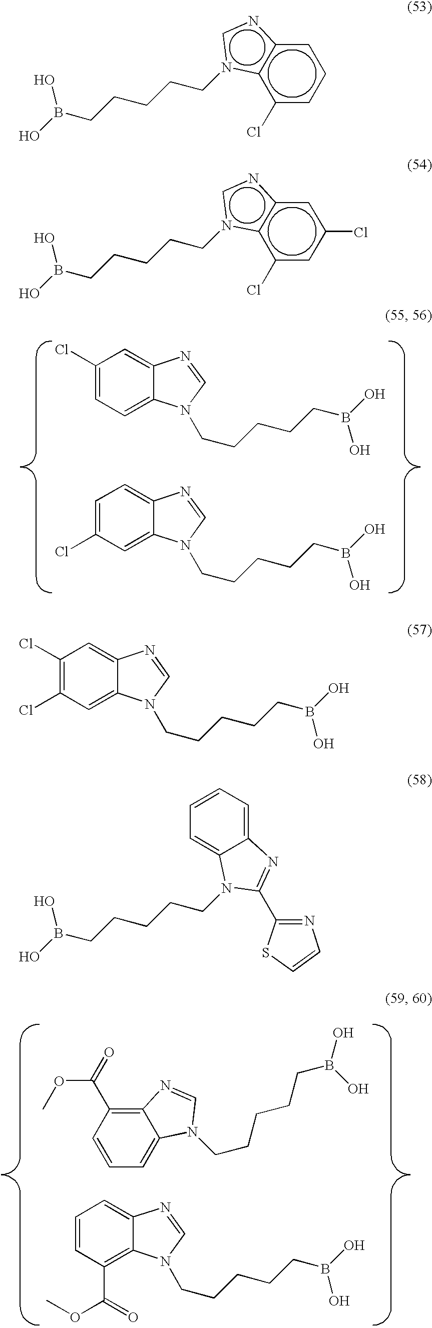

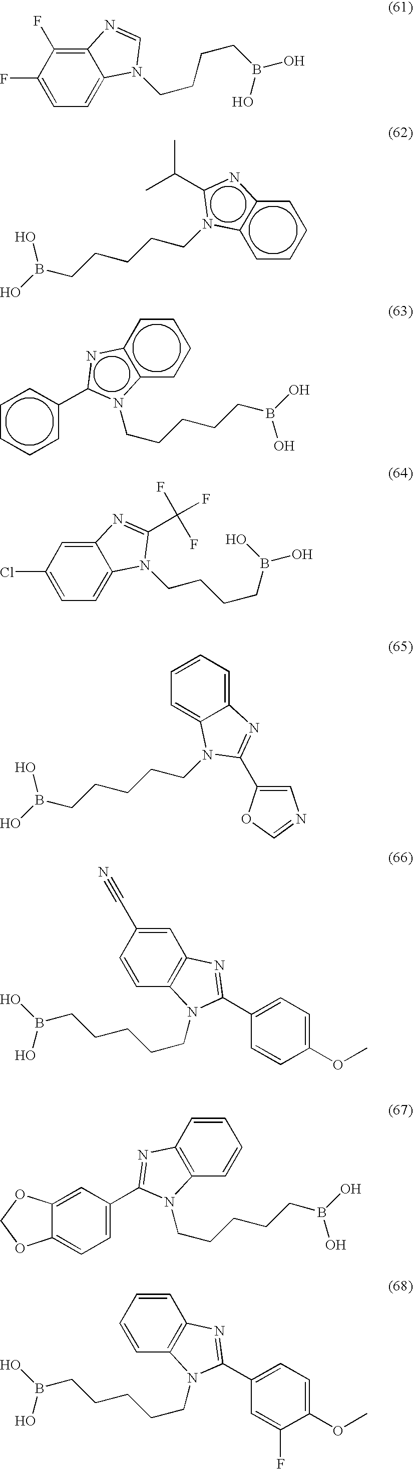

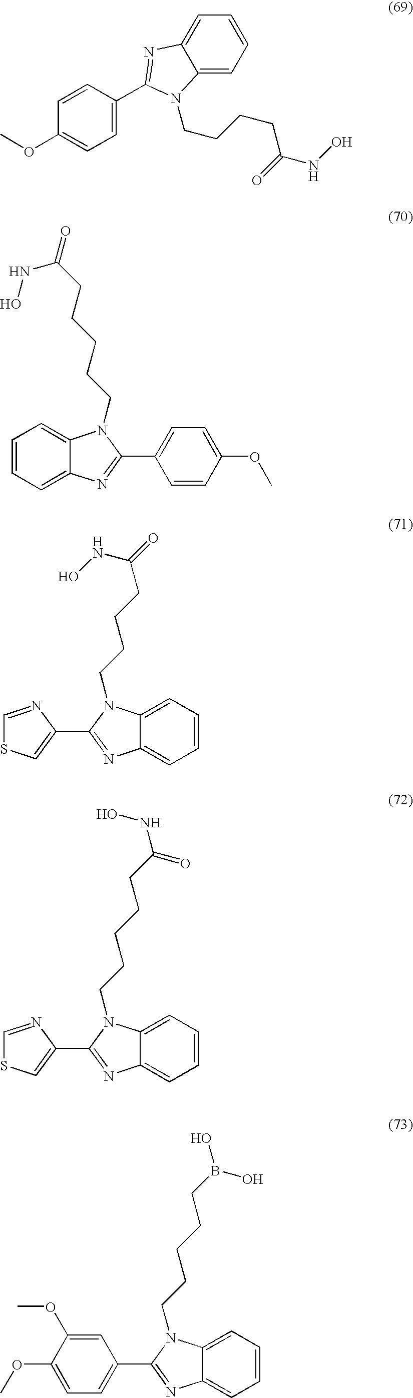

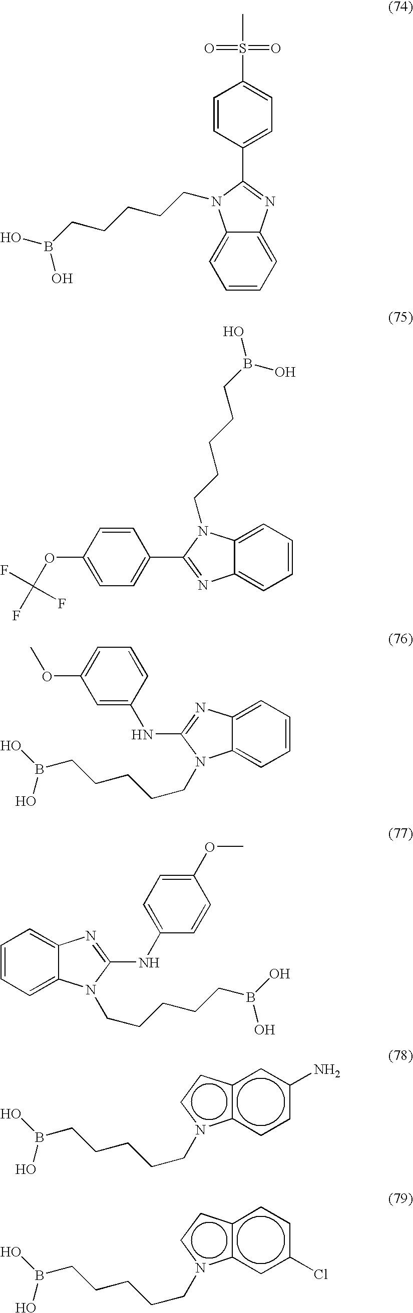

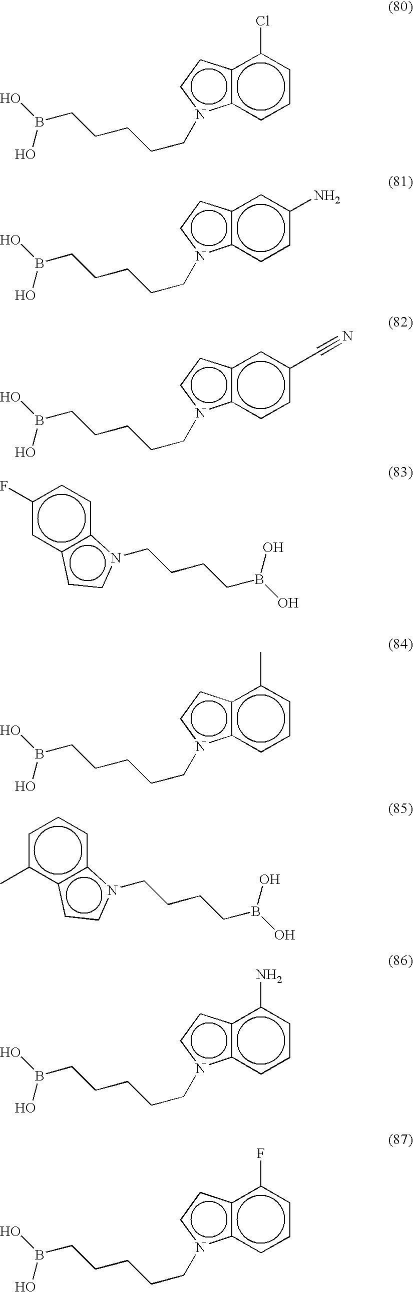

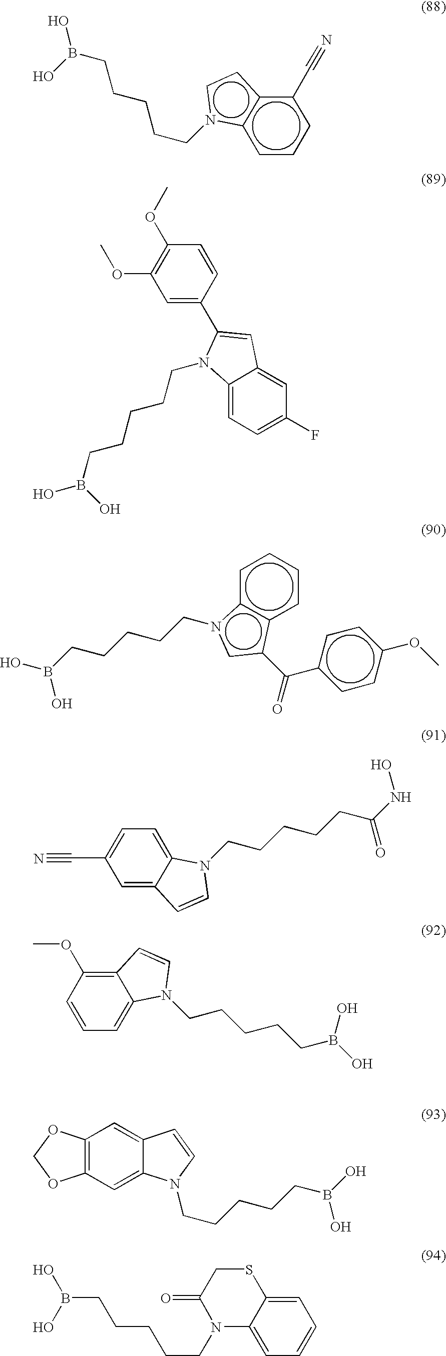

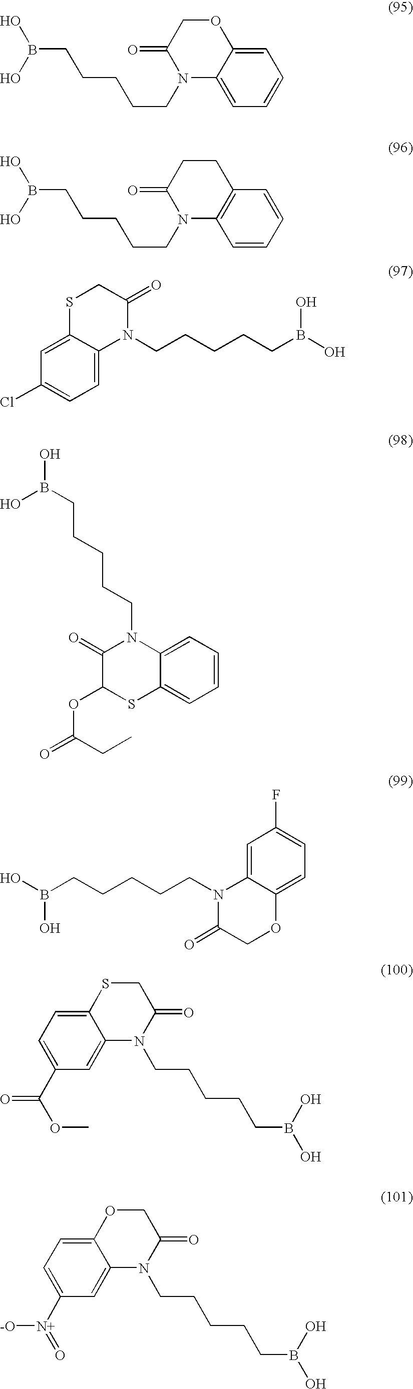

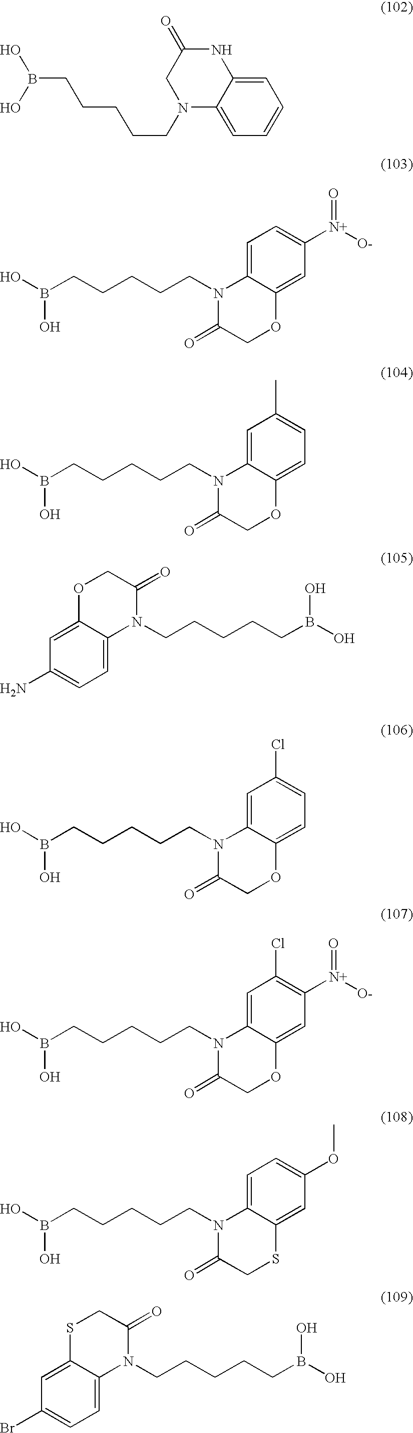

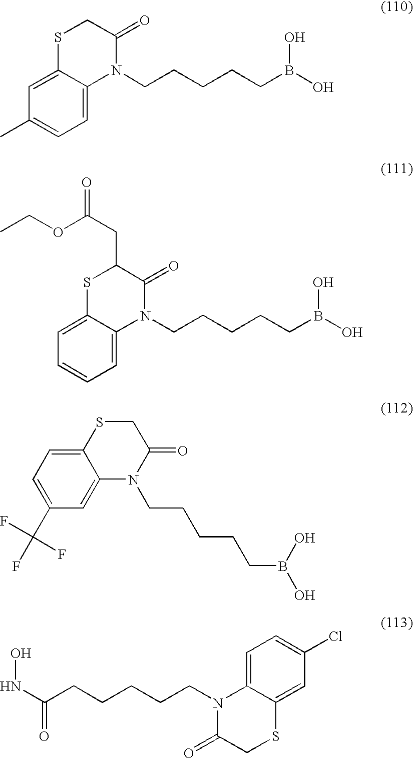

The active compounds disclosed herein can, as noted above, be prepared in the form of their pharmaceutically acceptable salts. Pharmaceutically acceptable salts are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; (b) salts formed from elemental anions such as chlorine, bromine, and iodine, and (c) salts derived from bases, such as ammonium salts, alkali metal salts such as those of sodium and potassium, alkaline earth metal salts such as those of calcium and magnesium, and salts with organic bases such as dicyclohexylamine and N-methyl-D-glucamine.

### 2. Pharmaceutical Formulations.

The active compounds described above may be formulated for administration in a pharmaceutical carrier in accordance with known techniques. See, inter alia, Remington: The Science and Practice of Pharmacy, 21^{th} Ed., Mack Publishing Co., Easton, Pa. (2006) and Handbook of Pharmaceutical Excipients, 3rd Ed, Kibbe, A. H. ed., Washington D.C., American Pharmaceutical Association (2000) hereby incorporated by reference in their entirety. In the manufacture of a pharmaceutical formulation according to the invention, the active compound (including the physiologically acceptable salts thereof) is typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.01 or 0.5% to 95% or 99% by weight of the active compound. One or more active compounds may be incorporated in the formulations of the invention, which may be prepared by any of the well known techniques of pharmacy consisting essentially of admixing the components, optionally including one or more accessory ingredients.

The formulations of the invention include those suitable for oral, rectal, topical, buccal (e.g., sub-lingual), vaginal, parenteral (e.g., subcutaneous, intramuscular, intradermal, or intravenous), topical (i.e., both skin and mucosal surfaces, including airway surfaces) and transdermal administration, although the most suitable route in any given case will depend on the nature and severity of the condition being treated and on the nature of the particular active compound which is being used.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations suitable for buccal (sub-lingual) administration include lozenges comprising the active compound in a flavoured base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the active compound, which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents. The formulations may be presented in unitdose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable, stable, sterile composition comprising a compound of Formula I, II, III, IV or V, or a salt thereof, in a unit dosage form in a sealed container. The compound or salt is provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent which is physiologically acceptable may be employed in sufficient quantity to emulsify the compound or salt in an aqueous carrier. One such useful emulsifying agent is phosphatidyl choline.

Formulations suitable for rectal administration are preferably presented as unit dose suppositories. These may be prepared by admixing the active compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof. In some embodiments, the compositions described herein can be administered from an inhaler through the mouth or nasal passage for pulmonary delivery.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and typically take the form of an optionally buffered aqueous solution of the active compound. Suitable formulations comprise citrate or bis\tris buffer (pH 6) or ethanol/water and contain from 0.1 to 0.2M active ingredient.

Further, the present invention provides liposomal formulations of the compounds disclosed herein and salts thereof. The technology for forming liposomal suspensions is well known in the art. When the compound or salt thereof is an aqueous-soluble salt, using conventional liposome technology, the same may be incorporated into lipid vesicles. In such an instance, due to the water solubility of the compound or salt, the compound or salt will be substantially entrained within the hydrophilic center or core of the liposomes. The lipid layer employed may be of any conventional composition and may either contain cholesterol or may be cholesterol-free. When the compound or salt of interest is water-insoluble, again employing conventional liposome formation technology, the salt may be substantially entrained within the hydrophobic lipid bilayer which forms the structure of the liposome. In either instance, the liposomes which are produced may be reduced in size, as through the use of standard sonication and homogenization techniques. Liposomal formulations containing the compounds disclosed herein or salts thereof, may be lyophilized to produce a lyophilizate which may be reconstituted with a pharmaceutically acceptable carrier, such as water, to regenerate a liposomal suspension.

Other pharmaceutical compositions may be prepared from the water-insoluble compounds disclosed herein, or salts thereof, such as aqueous base emulsions. In such an instance, the composition will contain a sufficient amount of pharmaceutically acceptable emulsifying agent to emulsify the desired amount of the compound or salt thereof. Particularly useful emulsifying agents include phosphatidyl cholines, and lecithin.

In addition to the active compounds, the pharmaceutical compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include methylparaben, propylparaben, and benzyl alcohol. The microbial preservative is typically employed when the formulation is placed in a vial designed for multidose use. Of course, as indicated, the pharmaceutical compositions of the present invention may be lyophilized using techniques well known in the art.

### 3. Subjects.

The present invention is primarily concerned with the treatment of human subjects, but the invention may also be carried out on animal subjects, particularly mammalian subjects such as mice, rats, dogs, cats, livestock and horses for veterinary purposes, and for drug screening and drug development purposes.

Subjects to be treated with active compounds, or administered active compounds, of the present invention are, in general, subjects in which an inflammatory cytokine such as tumor necrosis factor alpha (TNF-α) is to be inhibited, and/or in which a phosphodiesterase (PDE) such as phosphodiesterase II, III, IV, and/or V is to be inhibited.

Subjects in need of treatment with active agents as described herein include, but are not limited to, subjects afflicted with invasive diseases, infections, and inflammatory diseases or states, such as: septic shock, cachexia (or weight loss associated with chronic diseases such as Alzheimer's disease, cancer, or AIDS), rheumatoid arthritis, inflammatory bowel disease (including but not limited to Crohn's disease and ulcerative colitis), multiple sclerosis, congestive or chronic heart failure, psoriasis, asthma, non insulin-dependent diabetes mellitus, cerebral malaria, anemia associated with malaria, stroke, periodontitis, AIDS, and Alzheimer's disease. Subjects afflicted with such diseases are administered the active compound of the present invention (including salts thereof), alone or in combination with other compounds used to treat the said disease, in an amount effective to combat or treat the disease.

A particularly preferred category of diseases for treatment by the methods of the present invention are inflammatory diseases, or inflammations.

Exemplary inflammatory diseases also include, but are not limited to allergic rhinitis, allergic conjunctivitis, atopic dermatitis, eczema, and Behcet's disease.

While it is presently believed that the aforesaid diseases are treated by the inhibitory effect of the active compounds described herein on TNF-α production (and/or phosphodiesterase 4, kinases implicated in inflammation), applicants do not wish to be bound to any specific theory of the invention, and it is intended that the treatment of particular diseases described herein by active compounds described herein be encompassed by the present invention without regard to the underlying physiological mechanism by which such treatment is accomplished.

4. Dosage and routes of administration.

As noted above, the present invention provides pharmaceutical formulations comprising the active compounds (including the pharmaceutically acceptable salts thereof), in pharmaceutically acceptable carriers for oral, rectal, topical, buccal, parenteral, intramuscular, intradermal, or intravenous, inhalation and transdermal administration.

The therapeutically effective dosage of any specific compound, the use of which is in the scope of present invention, will vary somewhat from compound to compound, and patient to patient, and will depend upon the condition of the patient and the route of delivery. In general, a dosage from about 0.05 or 0.1 to about 20, 50 or 100 mg/kg subject body weight may be utilized to carry out the present invention. For example, a dosage from about 0.1 mg/kg to about 50 or 100 mg/kg may be employed for oral administration; or a dosage of about 0.05 mg/kg to 20 or 50 mg/kg, or more, may be employed for intramuscular injection. The duration of the treatment may be one or two dosages per day for a period of two to three weeks, or until the condition is controlled or treated. In some embodiments lower doses given less frequently can be used prophylactically to prevent or reduce the incidence of recurrence of the condition being treated.

The present invention is explained in greater detail in the following non-limiting Examples.

### Example 1

### 4-(2-(Trifluoromethyl)-1H-benzo[d]imidazol-1-yl)butylboronic acid

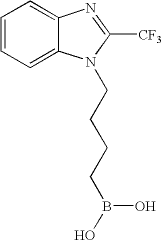

A 20 mL scintillation vial was charged with 2-(trifluoromethyl)benzimidazole (50 mg, 0.27 mmol, 1.0 equiv) and 95% sodium hydride (8 mg, 0.32 mmol, 1.2 equiv). Anhydrous dimethylformamide was added, and the reaction mixture was stirred for 10 min. A 1.0 M solution of 4-bromobutylboronic acid (53 mg, 0.30 mmol, 1.1 equiv) in dimethylformamide was added. The reaction was stirred at ambient temperature. After 5 days the reaction mixture was filtered through celite and concentrated in vacuo. The residue was purified by reverse-phase HPLC to afford 4-(2-(trifluoromethyl)-1H-benzo[d]imidazol-1-yl)butylboronic acid (43 mg, 53%): ¹H NMR (300 MHz, CD₃CN): δ 7.93 (d, J=8.0 Hz, 1H), 7.77 (d, J=8.0 Hz, 1H), 7.59 (t, J=7.4 Hz, 1H), 7.50 (m, 1H), 5.61 (s, 2H), 4.47 (t, J=7.7 Hz, 2H), 1.96 (pent, J=7.8 Hz, 2H), 1.57 (pent, J=7.8 Hz, 2H), 0.85 (t, J=7.9 Hz, 2H).

### Examples 2-4

### 5-(2-(Thiazol-4-yl)-1H-benzo[d]imidazol-1-yl)pentylboronic acid

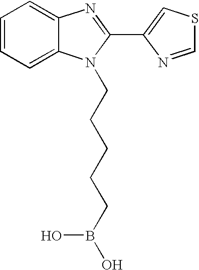

Cesium carbonate (486 mg, 1.50 mmol, 3.0 equiv) was added to a solution of thiabendazole (100 mg, 0.50 mmol, 1.0 equiv) in anhydrous dimethylformamide. After stirring for 10 min, a 1.0 M solution of 5-bromopentylboronic acid (145 mg, 0.75 mmol, 1.5 equiv) was added. The reaction mixture was stirred at ambient temperature. After 5 h, the reaction mixture was filtered. Silica gel diol (1.1 g, 3 equiv) was added to the filtrate and shaken for 30 min. The silica gel was washed with 30 mL of acetonitrile followed by 30 mL of 95:5 water-acetonitrile with 25 mmol trifluoroacetic acid. The aqueous wash was concentrated in vacuo, and the residue was purified by reverse-phase HPLC to afford 5-(2-(thiazol-4-yl)-1H-benzo[d]imidazol-1-yl)pentylboronic acid (110 mg, 70%).

A 1 dram vial was charged with thiabendazole (50 mg, 0.25 mmol, 1.0 equiv) and 95% sodium hydride (7.5 mg, 0.30 mmol, 1.2 equiv). Anhydrous dimethylformamide was added, and the reaction mixture was stirred for 10 min. A 1.0 M solution of 5-bromopentylboronic acid (53 mg, 0.27 mmol, 1.1 equiv) in anhydrous dimethylformamide was added, and the reaction mixture was stirred at ambient temperature. After 4 days the reaction mixture was filtered and concentrated in vacuo. The residue was purified by reverse-phase HPLC to afford 5-(2-(thiazol-4-yl)-1H-benzo[d]imidazol-1-yl)pentylboronic acid (10.0 mg, 13%): ¹H NMR (300 MHz, CD₃CN): δ 9.39 (br s, 1H), 8.73 (br s, 1H), 7.88 (m, 1H), 7.72 (m, 1H), 7.46 (m, 2H), 4.72 (t, J=7.6 Hz, 2H), 1.71 (m, 2H), 1.21 (m, 2H), 0.43 (t, J=6.9 Hz, 2H).

Thiabendazole (10 g, 49.75 mmol) was added to a suspension of cesium carbonate (48.5 g, 149 mmol, 3.0 equiv) in dimethylformamide. After stirring for 30 min, a solution of bromopentylboronic acid (15 g, 77 mmol) was added. The reaction mixture was stirred for 2 days, then DI water was added until precipitate formed, product was filtered, then washed with water and filtered again. White solid was dried via vacuum (15 g, yield 96%). ¹H NMR (300 MHz, d6-DMSO): δ 9.39 (br s, 1H), 8.73 (br s, 1H), 7.88 (m, 1H), 7.72 (m, 1H), 7.46 (m, 2H), 4.72 (t, J=7.6 Hz, 2H), 1.71 (m, 2H), 1.21 (m, 2H), 0.43 (t, J=6.9 Hz, 2H). Elemental analysis: C, 56.99%, H, 5.91%, N, 13.33%.

### Example 5

### 5-(5,6-dimethyl-1H-benzo[d]imidazol-1-yl)pentylboronic acid

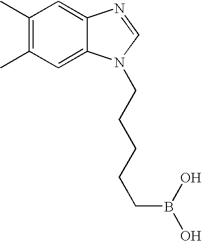

A suspension of 5,6-dimethylbenzimidazole (50 mg, 0.34 mmol) and potassium carbonate (70.9 mg, 0.51 mmol) in DMF (0.3 M) in a 40 mL scintillation vial was stirred for 30 min. A solution of 5-bromopentylboronic acid, (1 M, 0.0.38 mmol) was added and stirred at room temperature for 90 h. The reaction was filtered through celite and washed with DMF. The filtrate was evaporated and the residue was purified by HPLC to give 5-(5,6-dimethyl-1H-benzo[d]imidazol-1-yl)pentylboronic acid (12.4 mg, 14%). ¹H NMR (CD₃CN, 300 MHz) δ8.794 (s, 1H), 7.65 (s, 1H), 7.585 (s, 1H), 4.333 (t, 2H, J=7.4 Hz), 2.425 (s, 3H), 2.398 (s, 3H), 1.444-1.269 (m, 4H), 0.66 (t, 2H, J=7.5 Hz).

### Example 6

### 5-(1H-imidazo[4,5-c]pyridin-1-yl)pentylboronic acid

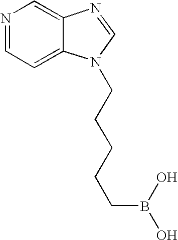

A suspension of 5-azabenzimidazole (50 mg, 0.42 mmol) and potassium carbonate (87.01 mg, 0.63 mmol) in DMF (0.3 M) in a 40 mL scintillation vial was stirred for 30 min. A solution of 5-bromopentylboronic acid, (1 M, 0.0.38 mmol) was added and stirred at room temperature for 90 h. The reaction was filtered through celite and washed with DMF. The filtrate was evaporated and the residue was purified by HPLC to give 5-(1H-imidazo[4,5-c]pyridin-1-yl)pentylboronic acid as a mixture of regioisomers (14.5 mg, 15%). ¹H NMR (CD₃CN) [symbol] 9.25 (s), 9.194 (s), 8.622 (s, 1H), 8.549-8.487 (m, 1H), 8.106 (d, J=6 Hz), 8.035 (d, J=6.3 Hz), 4.553 (t, J=7.4), 4.385 (p, J=7.1 Hz), 1.963-1.871 (m, 2H), 1.456-1.267 (m, 4H), 0.694-0.631 (m, 2H).

### Example 7

### 5-(2-(4-Methoxyphenyl)-1H-benzo[d]imidazol-1-yl)pentylboronic acid

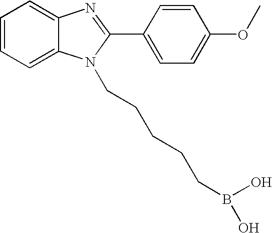

A 20 mL scintillation vial was charged with 2-(4-methoxyphenyl)-1H-benzo[d]imidazole (100 mg, 0.45 mmol, 1.0 eq), tetrabutylammonium iodide (16 mg, 0.04 mmol, 0.1 eq), and 95% sodium hydride (26 mg, 1.04 mmol, 2.3 eq). Tetrahydrofuran was added to the vial, and the reaction mixture was stirred until gas evolution was no longer evident. A 1.0 M solution 5-bromopentylboronic acid (96 mg, 0.49 mmol, 1.5 eq) was added via syringe. The reaction mixture was stirred on a J-chem shaker at 180 rpm. After 48 h the reaction mixture was concentrated in vacuo. The residue was purified using an ISCO combiflash (12 g SiO₂, 30 ml/min, ethyl acetate to 9:1 ethyl acetate-methanol). The appropriate fractions were concentrated in vacuo and the resulting oil was lyophilized from 3:1 acetonitrile-water to afford 5-(2-(4-methoxyphenyl)-1H-benzo[d]imidazol-1-yl)pentylboronic acid (53 mg, 35%) as a white powder: ¹H NMR (400 MHz, d₆-DMSO): δ 7.67 (m, 2H), 7.60 (m, 1H), 7.36 (s, 2H), 7.22 (m, 1H), 7.10 (m, 1H), 7.10 (m, 2H), 4.22 (t, J=7.3 Hz, 2H), 3.82 (s, 3H), 1.64 (pent, J=7.4 Hz, 2H), 1.22 (pent, J=7.6 Hz, 2H), 1.09 (m, 2H), 0.46 (t, J=7.6 Hz, 2H).

### Example 8

### 2-(3-Fluoro-4-methoxyphenyl)-1H-benzo[d]imidazole

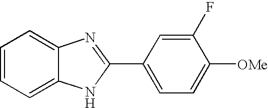

Samples of 3-fluoro-4-methoxybenzaldehyde (771 mg, 5 mmol) and 1,2-phenylenediamine (541 mg, 5 mmol) were suspended in nitrobenzene (2 mL) in a microwavable pressure tube (CEM). The mixture was subjected to microwave conditions (CEM Explorer, 200° C. and a hold time of 10 min). Upon cooling to room temperature, a large amount of a crystalline solid formed. The solid was filtered and triturated with hexane (3×20 mL) and hexane/EtOAc 4:1 (3×20 mL). The product was isolated as a tan solid (856 mg. 71%). ¹H NMR (400 MHz, CD₃CN): δ 7.84-7.89 (m, 2H), 7.60 (bs, 2 H), 7.22-7.27 (m, 3H), 3.96 (s, 3H).

### Example 9

### 2-(5-Bromopentyl)-4,4,5,5-tetramethyl-1,3,2-dioxaborolane

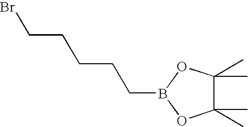

A solution of 5-bromopentylboronic acid (9.75 g, 50 mmol) and pinacol (5.91 g, 50 mmol) in acetonitrile (125 mL) was stirred at room temperature for 16 hr. The reaction mixture was concentrated under reduced pressure to give a dark gray residue. Purification using an Isco purification system (silica column, eluted with hexane/EtOAc 4:1) gave the product as a clear liquid (8.1 g, 58%). Visualization of the product in TLC analysis was achieved using anisaldehyde or KMnO₄ staining followed by heating. ¹H NMR (400 MHz, CD₃CN): [symbol] 3.48 (t, J=6.8 Hz, 2H), 1.82-1.86 (m, 2H), 1.40-1.42 (m, 4 H), 1.23 (s, 12H), 0.71-0.75 (m, 2H).

### Example 10

### 2-(3-Fluoro-4-methoxyphenyl)-1-(5-(4,4,5,5-tetramethyl-1,3,2-dioxaborolan-2-yl)pentyl)-1H-benzo[d]imidazole

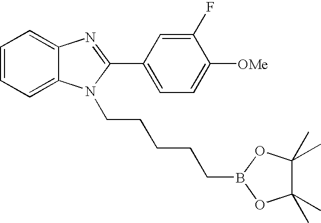

A suspension of 2-(3-fluoro-4-methoxyphenyl)-1H-benzo[d]imidazole (300 mg, 1.24 mmol), 2-(5-bromopentyl)-4,4,5,5-tetramethyl-1,3,2-dioxaborolan (687 mg, 2.48 mmol) and cesium carbonate (808 mg, 2.48 mmol) in DMF (2.5 mL) was stirred at room temperature for 22 hr. The reaction mixture was diluted with EtOAc (25 mL) and H₂O (25 mL). The organic phase was extracted with aqueous LiCl (10%, 25 mL). The organic phase was dried (Na₂SO₄). The solvent was removed to afford a brown residue. Purification using an Isco purification system (silica column, eluted with hexane/EtOAc 4:1) gave the product as a clear liquid (8.1 g, 58%). ¹H NMR (400 MHz, CD₃CN): δ 7.53-7.68 (m, 1H), 7.50-7.52 (m, 3H), 7.24-7.30 (m, 3H), 4.24-4.28 (m, 2H), 3.96 (m, 3H), 1.71-1.75 (m, 2H), 1.10-1.30 (m, 16H), 0.58-0.62 (m, 2H).

### Example 11

### 5-(2-(3-Fluoro-4-methoxyphenyl)-1H-benzo[d]imidazol-1-yl)pentylboronic acid

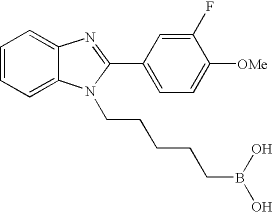

Samples of 2-(3-fluoro-4-methoxyphenyl)-1-(5-(4,4,5,5-tetramethyl-1,3,2-dioxaborolan-2-yl)pentyl)-1H-benzo[d]imidazole (810 mg, 1.85 mmol) and diethanolamine (2.1 g, 20 mmol) were combined in a microwavable pressure tube (CEM). The mixture was subjected to microwave conditions (CEM Explorer, 60° C. and a hold time of 10 min). LC-MS analysis showed some starting material. Another portion of diethanolamine (2.1 g, 20 mmol) was added to the viscous mixture. The mixture was again subjected to microwave conditions (60° C. and a hold time of 10 min). LC-MS analysis showed a trace of the starting material remaining. Thus, the reaction mixture was diluted with H₂O (50 mL) to form an emulsion. Extraction was performed sequentially using hexane (50 mL), hexane/EtOAc 4:1 (3×50 mL) and ether (2×50 mL). To the aqueous phase was added HCl (1M aqueous, 100 mL) followed by CH₂Cl₂ (100 mL). The mixture was stirred at room temperature for 20 min. The pH of the aqueous phase was adjusted to 8 using solid K₂CO₃. The organic phase was separated. The aqueous phase was extracted with CH₂Cl₂/EtOH 3:1 (3×100 mL). The organic phase was combined and dried (MgSO₄). The solvent was removed under reduced pressure to give an oily residue. Acetonitrile/H₂O 1:1 (20 mL) was added to the residue. After thorough mixing and solvent removal, an off-white solid was obtained. Trituration with hexane/EtOAc 4:1 (3×50 mL) afforded the material slightly contaminated with 2-(3-fluoro-4-methoxyphenyl)-1-(5-(4,4,5,5-tetramethyl-1,3,2-dioxaborolan-2-yl)pentyl)-1H-benzo[d]imidazole. The solid was then dissolved in acetone (5 mL) with heating. After cooling, the addition of hexane (30 mL) induced the precipitation of a white solid (250 mg, 38%). ¹H NMR 400 MHz, CD₃CN): δ 7.67-7.69 (m, 1H), 7.50-7.56 (m, 3H), 7.23-7.33 (m, 3H), 4.27 (t, J=8.0 Hz, 2H), 3.97 (s, 3H), 1.72-1.80 (m, 2H), 1.15-1.34 (m, 4 H), 0.60 (t, J=8.0 Hz, 2H).

### Example 12

### ethyl 6-(2-(thiazol-4-yl)-1H-benzo[d]imidazol-1-yl)hexanoate

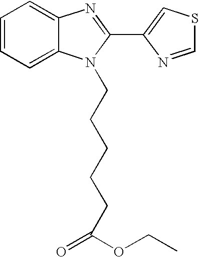

Cesium carbonate (2425 mg, 7.5 mmol, 3.0 equiv) was added to a solution of thiabendazole (500 mg, 2.48 mmol, 1.0 equiv) in anhydrous dimethylformamide. After stirring for 30 min, a solution of ethyl 5-bromohexanoate (1106 mg, 4.96 mmol, 2 equiv) was added. The reaction mixture was stirred for 3 hours. Then water (8:1) was added and this was extracted with ethyl acetate. The ethyl acetate solution was concentrated in vacuo and the residue was purified by silica gel column using ethyl acetate/hexane as an eluting solvent to afford ethyl 6-(2-(thiazol-4-yl)-1H-benzo[d]imidazol-1-yl)hexanoate.

(650 mg, 76%): ¹H NMR (300 MHz, d6-DMSO): δ 9.32 (d, J=1.76 Hz, 1H), 8.48 (d, J=1.76 Hz, 1H), 7.64 (t,d, J=7.03 Hz, 1.7 Hz 2H), 7.25 (m, 2H), 4.72 (t, J=7.3 Hz, 2H), 3.99 (q, J=7.03 Hz, 2H), 2.19 (t, J=7.3 Hz, 2H), 1.73 (pent, J=7.3 Hz, 2H), 1.476 (pent, J=7.62 Hz, 2H), 1.23 (m, 2H), 1.106 (t, J=7.03 Hz, 3H).

### Example 13

### N-hydroxy-6-(2-(thiazol-4-yl)-1H-benzo[d]imidazol-1-yl)hexanamide

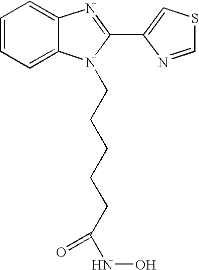

To a neat ethyl 6-(2-(thiazol-4-yl)-1H-benzo[d]imidazol-1-yl)hexanoate (400 mg, 1.16 mmol) N,O-Bis(trimethylsilyl)hydroxylamine (5,8 mmol, 1.03 g, 5 eq.) was added at room temperature. After stirring for 30 min a solution of 1N NaOH (2 ml) was added followed by the addition of methanol (˜7 ml). Then reaction mixture was concentrated via rotovap and then purified on silica gel column using methylene chloride/methanol as an eluting solvent (121 mg, 31%): ¹H NMR (300 MHz, d6-DMSO): δ 10.27 (s, 1H), 9.32 (d, J=2.345 Hz, 1H), 8.637 (s, 1H), 8.48 (d, J=1.759 Hz, 1H), 7.637 (t, J=8.793 Hz, 2H), 7.25 (m, 2H), 4.70 (t, J=7.33 Hz, 2H), 1.862 (t, J=7.33 Hz, 2H), 1.717 (t, J=7.33 Hz, 2H), 1.452 (t, J=7.33 Hz, 2H), 1.219 (m, 2H).

### Example 14

### ethyl 5-(2-(thiazol-4-yl)-1H-benzo[d]imidazol-1-yl)pentanoate

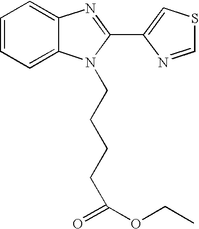

¹H NMR (300 MHz, d6-DMSO): δ 9.32 (d, J=1.759 Hz, 1H), 8.489 (d, J=2.345 Hz, 1H), 7.643 (t, J=6.741 Hz, 2H), 7.25 (m, 2H), 4.748 (t, J=7.034 Hz, 2H), 3.98 (q, J=7.6 Hz, 2H), 3.513 (t, J=6.448 Hz, 2H), 1.610 (pent, J=7.33 Hz, 2H), 1.477 (pent, J=7.622 Hz, 2H), 1.087 (t, J=7.034 Hz, 3H).

### Example 15

### N-hydroxy-5-(2-(thiazol-4-yl)-1H-benzo[d]imidazol-1-yl)pentanamide

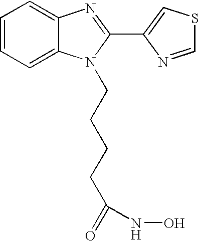

¹H NMR (300 MHz, d6-DMSO): δ 10.34 (broad, 1H), 9.438 (s, 1H), 8.754 (s, 1H), 7.88 (d, J=8.2 Hz, 1H), 7.76 (d, J=8.2 Hz, 1H), 7.47 (pent, J=5.5 Hz, 2H), 4.8 (t, J=7.034 Hz, 2H), 1.95 (t, J=7.3 Hz, 2H), 1.79 (pent, J=7.3 Hz, 2H), 1.52 (pent, J=7.62 Hz, 2H).

### Example 16

### ethyl 5-(2-(4-methoxyphenyl)-1H-benzo[d]imidazol-1-yl)pentanoate

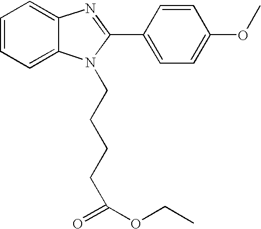

¹H NMR (300 MHz, d6-DMSO): δ 7.68 (d, J=8.79 Hz, 2H), 7.6 (m, 2H), 7.2 (m, 2H), 7.1 (d, J=8.79 Hz, 2H), 4.27 (t, J=7.3 Hz, 2H), 3.95 (q, J=7.034 Hz, 2H), 3.83 (s, 3H), 2.178 (t, J=7.3 Hz, 2H), 1.67 (m, 2H), 1.37 (pent, J=7.620 Hz, 2H), 1.096 (t, J=7.034 Hz, 3H).

### Example 17

### N-hydroxy-5-(2-(4-methoxyphenyl)-1H-benzo[d]imidazol-1-yl)pentanamide

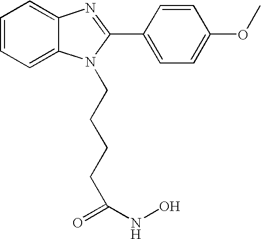

¹H NMR (300 MHz, d6-DMSO): δ 8.05 (d, J=7.62 Hz, 1H), 7.8 (d, J=8.79 Hz, 4H), 7.6 (m, 2H), 7.25 (d, J=8.79 Hz, 2H), 4.43 (t, J=7.3 Hz, 2H), 3.88 (s, 3H) 1.88 (t, J=7.034 Hz, 2H), 1.74 (m, 2H), 1.44 (pent, J=7.62 Hz, 2H).

### Example 18

### ethyl 6-(2-(4-methoxyphenyl)-1H-benzo[d]imidazol-1-yl)hexanoate

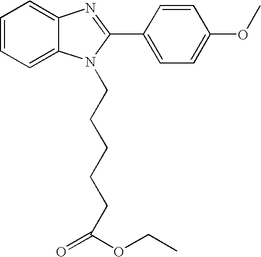

¹H NMR (300 MHz, d6-DMSO): δ 7.68 (d, J=8.79 Hz, 2H), 7.6 (m, 2H), 7.2 (m, 2H), 7.1 (d, J=8.79 Hz, 2H), 4.26 (q, J=7.3 Hz, 2H), 3.98 (m, 2H), 3.83 (s, 3H), 2.137 (t, J=7.3 Hz, 2H), 1.67 (m, 2H), 1.37 (m, 2H), 1.1 (m, 5H).

### Example 19

### N-hydroxy-6-(2-(4-methoxyphenyl)-1H-benzo[d]imidazol-1-yl)hexanamide

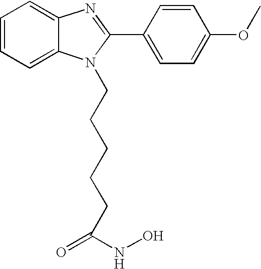

¹H NMR (300 MHz, d6-DMSO): δ 10.311 (broad, 1H), 7.856 (d, J=7.03 Hz, 1H), 7.76 (m, 3H), 7.433 (pent, J=5.8 Hz, 2H), 7.209 (d, J=8.79 Hz, 2H), 4.322 (t, J=7.3 Hz, 2H), 3.865 (s, 3H), 1.842 (t, J=7.3 Hz, 2H), 1.717 (pent, J=7.034 Hz, 2H), 1.385 (pent, J=7.3 Hz, 2H), 1.147 (m, 2H).

### Example 20

### 5-(5-cyano-1H-indol-1-yl)pentylboronic acid

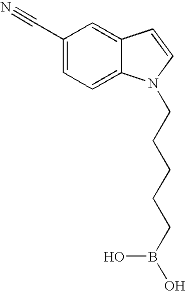

A 1 dram vial was charged with 5-cyanoindole (50 mg, 0.35 mmol, 1.0 equiv) and 95% sodium hydride (10.6 mg, 0.42 mmol, 1.2 equiv). Anhydrous dimethylformamide was added, and the reaction mixture was stirred for 10 min. A 1.0 M solution of 5-bromopentylboronic acid (75.4 mg, 0.39, 1.1 equiv) in dimethylformamide was added, and the reaction mixture was stirred at ambient temperature. After 4 days the reaction mixture was filtered and concentrated in vacuo. The residue was purified by reverse-phase HPLC to afford 5-(5-cyano-1H-indol-1-yl)pentylboronic acid (46.5 mg, 52%): ¹H NMR (300 MHz, CD₃CN): δ 7.99 (s, 1H), 7.54 (d, J=8.9 Hz, 1H), 7.36-7.45 (m, 2H), 6.58 (d, J=2.7 Hz, 1H), 4.18 (t, J=7.16 Hz, 2H), 1.79 (pent, J=7.3 Hz, 2H), 1.38 (m, 2H), 1.23 (m, 2H), 0.64 (t, J=7.6 Hz, 2H).

### Example 21

### 5-(4-Cyano-1H-indol-1-yl)pentylboronic acid

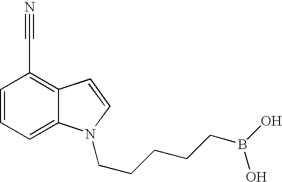

A 1 dram vial was charged with 4-cyanoindole (51 mg, 0.36 mmol, 1.0 equiv) and 95% sodium hydride (20.9 mg, 0.83 mmol, 2.3 equiv). Anhydrous dimethylformamide was added, and the reaction mixture was stirred for 10 min. A 1.0 M solution of 5-bromopentylboronic acid (76.9 mg, 0.39 mmol, 1.1 equiv) in dimethylformamide was added, and the reaction mixture was stirred at ambient temperature. After 2 days the reaction mixture was filtered and concentrated in vacuo. The residue was purified by reverse-phase HPLC to afford 5-(4-cyano-1H-indol-1-yl)pentylboronic acid: ¹H NMR (300 MHz, CD₃CN): δ 7.71 (m, 1H), 7.45 (m, 2H), 7.26 (t, J=8.0 Hz, 1H), 6.61 (d, J=2.1 Hz, 1H), 4.19 (t, J=7.15 Hz, 2H), 1.80 (pent, J=7.3 Hz, 2H), 1.38 (m, 2H), 1.24 (m, 2H), 0.64 (t, J=7.4 Hz, 2H).

### Example 22

### 5-(2-Methyl-1H-indol-1-yl)pentylboronic acid

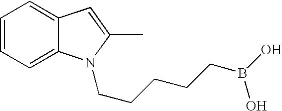

A 1 dram vial was charged with 2-methylindole (50 mg, 0.38 mmol, 1.0 equiv) and 95% sodium hydride (22.0 mg, 0.87 mmol, 2.3 equiv). Anhydrous dimethylformamide was added, and the reaction mixture was stirred for 10 min. A 1.0 M solution of 5-bromopentylboronic acid (81.1 mg, 0.42 mmol, 1.1 equiv) in dimethylformamide was added, and the reaction mixture was stirred at ambient temperature. After 2 days the reaction mixture was filtered and concentrated in vacuo. The residue was purified by reverse-phase HPLC to afford 5-(2-Methyl-1H-indol-1-yl)pentylboronic acid: ¹H NMR (300 MHz, CD₃CN): δ 7.43 (d, J=7.7 Hz, 1H), 7.31 (d, J=8.25 Hz, 1H), 7.07 (m, 1H), 6.97 (m, 1H), 6.18 (s, 1H), 4.07 (m, 2H), 2.40 (s, 3H), 1.69 (m, 2H), 1.35 (m, 4H), 0.66 (m, 2H).

### Example 23

### 5-(4-Fluoro-1H-indol-1-yl)pentylboronic acid

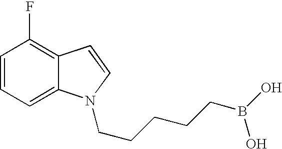

A 1 dram vial was charged with 4-fluoroindole (50 mg, 0.37 mmol, 1.0 equiv) and 95% sodium hydride (21.5 mg, 0.85 mmol, 2.3 equiv). Anhydrous dimethylformamide was added, and the reaction mixture was stirred for 10 min. A 1.0 M solution of 5-bromopentylboronic acid (79.3 mg, 0.41 mmol, 1.1 equiv) in dimethylformamide was added, and the reaction mixture was stirred at ambient temperature. After 2 days the reaction mixture was filtered and concentrated in vacuo. The residue was purified by reverse-phase HPLC to afford 5-(2-Methyl-1H-indol-1-yl)pentylboronic acid: ¹HNMR (300 MHz, CD₃CN): [symbol] 7.21 (m, 2H), 7.10 (m, 1H), 6.74 (m, 1H), 6.49 (m, 1H), 4.13 (m, 2H), 1.79 (m, 2H), 1.38 (m, 2H), 1.27 (m, 2H), 0.65 (m, 2H).

### Example 24

### 5-(4-Amino-1H-indol-1-yl)pentylboronic acid

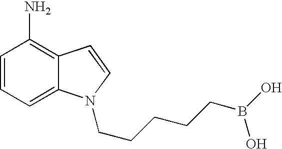

A 1 dram vial was charged with 4-aminoindole (51 mg, 0.39 mmol, 1.0 equiv) and 95% sodium hydride (22.4 mg, 0.89 mmol, 2.3 equiv). Anhydrous dimethylformamide was added, and the reaction mixture was stirred for 10 min. A 1.0 M solution of 5-bromopentylboronic acid (82.7 mg, 0.42 mmol, 1.1 equiv) in dimethylformamide was added, and the reaction mixture was stirred at ambient temperature. After 2 days the reaction mixture was filtered and concentrated in vacuo. The residue was purified by reverse-phase HPLC to afford 5-(2-Methyl-1H-indol-1-yl)pentylboronic acid: ¹H NMR (300 MHz, CD₃CN): δ 7.19 (d, J=3.3 Hz, 1H), 7.08 (m, 2H), 6.63 (d, J=7.2 Hz, 1H), 6.48 (d, J=2.8 Hz, 1H), 4.12 (t, J=7.2 Hz, 2H), 1.78 (pent, J=7.3 Hz, 2H), 1.38 (m, 2H), 1.25 (m, 2H), 0.64 (t, J=7.7 Hz, 2H).

### Example 25

### 5-(5-Fluoro-2-(3,4-dimethoxyphenyl)-1H-indol-1-yl)pentylboronic acid

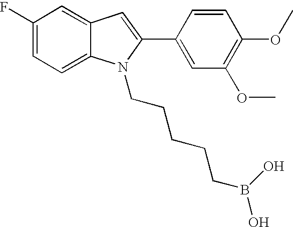

Sodium hydride (60 wt % dispersion in mineral oil, 81 mg, 2.02, 1.1 equiv) was added to a solution of 5-fluoro-2-(3,4-dimethoxyphenyl)-1H-indole (500 mg, 1.84 mmol, 1.0 equiv) in 8.2 mL of anhydrous dimethylformamide. The resulting yellow reaction mixture was stirred 10 min at ambient temperature. A solution of 2-(5-bromopentyl)-4,4,5,5-tetramethyl-1,3,2-dioxaborolane (561 mg, 2.02 mmol, 1.1 equiv) in 1.0 mL of anhydrous dimethylformamide was added via syringe. After 2 h the reaction mixture was partitioned with 200 mL of 1:1 water-ethyl acetate. The layers were separated, and the aqueous layer was extracted with ethyl acetate (2×100 mL). The combined organic layers were washed with aqueous lithium chloride and brine, dried over sodium sulfate, filtered, and concentrated in vacuo. The residue was purified on an ISCO combiflash (40 g SiO₂, 40 mL/min, 4:1 hexanes-ethyl acetate) to afford 5-fluoro-2-(3,4-dimethoxyphenyl)-1-(5-(4,4,5,5-tetramethyl-1,3,2-dioxaborolan-2-yl)pentyl)-1H-indole as a clear oil.

[symbol]

A solution/suspension of 5-fluoro-2-(3,4-dimethoxyphenyl)-1-(5-(4,4,5,5-tetramethyl-1,3,2-dioxaborolan-2-yl)pentyl)-1H-indole (114.5 mg, 0.245 mmol, 1.0 equiv) and diethanolamine (47 □L, 0.490 mmol, 2.0 equiv) in 5.0 mL of diethyl ether was heated at 40° C. After 20 h the milky reaction mixture was cooled to ambient temperature, and the precipitate was collected by filtration. The solids were washed with diethyl ether. The pasty white solid was stirred for 20 min in 10 mL of 1:1 dichloromethane-1 N aqueous hydrochloric acid. The layers were separated, and the aqueous layer was extracted with dichloromethane (4×10 mL). The combined organic layers were washed with saturated aqueous ammonium chloride, dried over sodium sulfate, filtered, and concentrated in vacuo. The residue was taken up in 3:1 acetonitrile-water and lyophilized to afford 5-(5-fluoro-2-(3,4-dimethoxyphenyl)-1H-indol-1-yl)pentylboronic acid as a white powder: ¹H NMR (400 MHz, CD₃CN): δ 7.49 (m, 1H), 7.27 (d, J=9.8 Hz, 2H), 7.07 (br s, 3H), 6.98 (t, J=9.6 Hz, 1H), 4.19 (t, J=6.1, 2H), 3.89 (s, 3H), 3.87 (s, 3H), 1.65 (m, 2H), 1.24 (m, 2H), 1.14 (m, 2H), 0.58 (m, 2H).

### Example 26

### ethyl 6-(5-cyano-1H-indol-1-yl)hexanoate

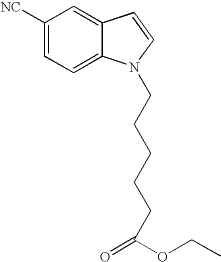

Cyanoindole (500 mg, 3.52 mmol) was added to a suspension of sodium hydride (1.1 eq. 148 mg of 60% dispersion in mineral oil) in dimethylformamide and the reaction was stirred for 10 min. Then ethyl 6-bromohexanoate (1.5 eq, 1.18 g, 5.28 mmol) was added dropwise. The reaction was stirred at ambient temperature for 5 hours. Then water (8:1) added and this was extracted with ethyl acetate. The ethyl acetate solution was concentrated in vacuo and the residue was purified by silica gel column using ethyl acetate/hexane as an eluting solvent to afford ethyl 6-(5-cyano-1H-indol-1-yl)hexanoate (850 mg, 85% yield). ¹H NMR (300 MHz, d6-DMSO): δ 8.057 (d, J=1.172 Hz, 1H), 7.65 (d, J=8.793 Hz, 1H), 7.6 (d, J=2.93 Hz, 1H), 7.5 (dd, J=1.759 Hz, 8.79 Hz, 1H), 6.6 (d, J=3.5 Hz, 1H), 4.2 (t, J=7.03 Hz, 2H), 3.98 (q, J=7.034 Hz, 2H), 2.2 (t, J=7.3, 2H), 1.72 (pent, J=7.62 Hz, 2H), 1.5 (pent, J=7.62 Hz, 2H), 1.2 (m, 2H), 1.1 (t, J=7.034 Hz, 3H).

### Example 27

### 6-(5-cyano-1H-indol-1-yl)-N-hydroxyhexanamide

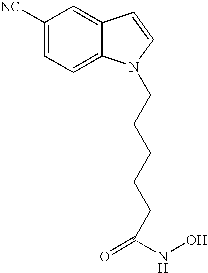

To a neat ethyl 6-(5-cyano-1H-indol-1-yl)hexanoate (850 mg, 2.99 mmol) N,O-Bis(trimethylsilyl)hydroxylamine (14.95 mmol, 2.65 g, 5 eq.) was added at room temperature. After stirring for 30 min solution of 1N NaOH (4 ml) was added followed by the addition of methanol. Then the reaction mixture was concentrated via rotovap and then purified on silica gel column using methylene chloride methanol as an eluting solvent to afford 310 mg (38% yield) of 6-(5-cyano-1H-indol-1-yl)-N-hydroxyhexanamide. ¹H NMR (300 MHz, d6-DMSO): δ 10.298 (broad, 1H), 8.656 (broad, 1H), 8.067 (d, J=1.172 Hz, 1H), 7.675 (d, J=8.207 Hz, 1H), 7.581 (d, J=3.517 Hz, 2H), 7.465 (d,d, J=8.79 Hz, 1.172 Hz, 2H), 6.578 (d, J=2.931 Hz, 1H), 4.197 (t, J=7.034 Hz, 2H), 1.88 (t, J=7.3 Hz, 2H), 1.717 (pent, J=7.3 Hz, 2H), 1.476 (pent, J=7.620 Hz, 2H), 1.167 (m, 2H).

### Example 28

### Synthesis of 2-substituted-2H-benzo[b][1,4]thiazin-3(4H)-ones

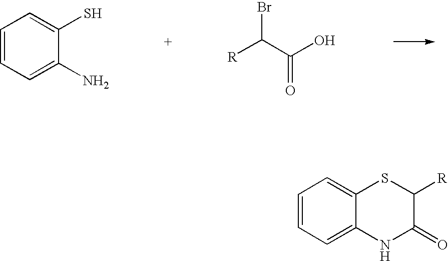

The thioaniline (1 mmol) and α-bromo-α-substituted acetic acid (0.9 mmol) is combined in xylenes (5.0 mL) and heated to 100° C. for six hours. After cooling the solvent is removed under reduced pressure, and the target product is purified on an HPLC-MS apparatus (Agilent) by mass directed fractionation.

### Example 29

### Synthesis of 5-(#-R-2,3-dihydro-3-oxobenzo[b][1,4](thia/oxe)zin-4-yl)pentylboronic acid

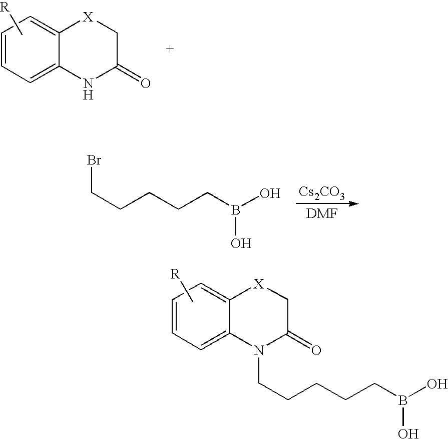

The parent ring (1.0 mmol), 5-bromo-1-pentylboronic acid (2.0 mmol), and cesium carbonate (2.5 mmol) are combined in 2.0 mL of DMF and shaken at ambient temperature for 48 hours. Alternatively, the 5-bromo-1-pentylboronic acid is added in 0.5 mmol aliquots every 12 hours for 48 hours. This increases both the conversion and yield. The reaction mixture is then filtered to remove the cesium carbonate, and the solvent is removed under reduced pressure. The target product is purified on an HPLC-MS apparatus (Agilent) by mass directed fractionation.

### Example 30

### 5-(6-fluoro-2,3-dihydro-3-oxobenzo[b][1,4]oxazin-4-yl)pentylboronic acid

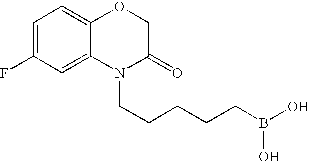

¹H NMR (400 MHz, CD₃CN): 6.98 (1H, dd), 6.92 (1H, dd), 6.75 (1H, dt), 4.55 (2H, s), 3.88 (2H, t), 1.62 (2H, m), 1.43 (2H, m), 1.34 (2H, m), 0.70 (2H, t).

### Example 31

### 5-(2,3-dihydro-3-oxobenzo[b][1,4]thiazin-4-yl)pentylboronic acid

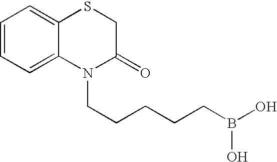

¹H NMR (400 MHz, CD₃CN): 7.41 (1H, m), 7.28 (2H, m), 7.05 (1H, m), 4.71 (2H, s), 3.37 (2H, m), 1.54 (2H, m), 1.34 (2H, m), 0.91 (2H, m), 0.70 (2H, t).

### Example 32

### 5-(7-chloro-2,3-dihydro-3-oxobenzo[b][1,4]thiazin-4-yl)pentylboronic acid

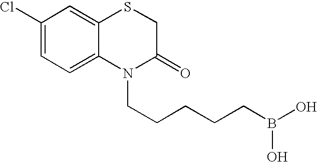

¹H NMR (400 MHz, CD₃CN): 7.44 (1H, d), 7.27 (1H, d), 7.22 (1H, s), 3.96 (2H, t), 3.39 (2H, s), 1.57 (2H, m), 1.38 (2H, m), 1.28 (2H, m), 0.67 (2H, t).

### Example 33

### 5-(2,3-dihydro-7-nitro-3-oxobenzo[b][1,4]oxazin-4-yl)pentylboronic acid

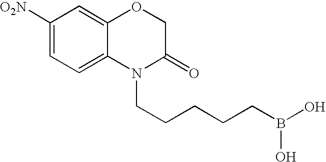

¹H NMR (400 MHz, CD₃CN): 7.92 (2H, m), 7.12 (1H, d), 4.73 (2H, s), 3.99 (2H, t), 1.66 (2H, m), 1.40 (4H, m), 0.71 (2H, t).

### Example 34

### 5-(2,3-dihydro-3-oxobenzo[b][1,4]oxazin-4-yl)pentylboronic acid

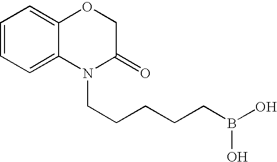

¹H NMR (400 MHz, CD₃CN): 7.13 (1H, d), 7.06 (2H, m), 7.00 (1H, m), 4.56 (2H, s), 3.91 (2H, t), 1.62 (2H, t), 1.38 (4H, m), 0.70 (2H, t).

### Example 35

### ethyl 2-(3,4-dihydro-3-oxo-4-(5-pentylboronic acid)-2H-benzo [b][1,4]thiazin-2-yl)acetate

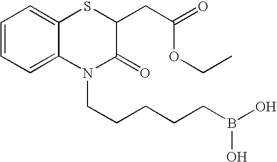

¹H NMR (400 MHz, CD₃CN): 7.41 (1H, dd), 7.31 (2H, m), 7.08 (1H, dt), 4.13 (2H, q), 3.97 (1H, dd), 3.81 (2H, 7), 2.89 (1H, dd), 2.54 (1H, dd) 1.57 (2H, m), 1.34 (4H, m), 1.23 (3H, t), 0.67 (2H, t).

### Example 36

### ethyl 6-(7-chloro-2,3-dihydro-3-oxobenzo[b][1,4]thiazin-4-yl)hexanoate

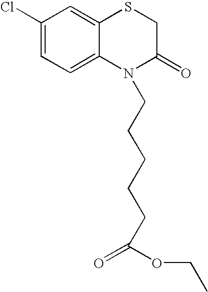

Cesium carbonate (2443 mg, 7.5 mmol, 3.0 equiv) was added to a solution of 7-chloro-2H-1,4-benzothiazin-3(4H)-one (500 mg, 2.5 mmol, 1.0 equiv) in anhydrous dimethylformamide. After stirring for 30 min, a solution of ethyl 5-bromohexanoate (1106 mg, 4.96 mmol, 2 equiv) was added. The reaction mixture was stirred for 3 hours. Then water (8:1) was added and this was extracted with ethyl acetate. The ethyl acetate solution was concentrated in vacuo and the residue was purified by silica gel column using ethyl acetate/hexane as an eluting solvent to afford ethyl 6-(7-chloro-3-oxo-2,3-dihydrobenzo[b][1,4]thiazin-4-yl)hexanoate (545 mg, 64% yield). ¹H NMR (300 MHz, d6-DMSO): δ 7.511 (m, 1H), 7.3 (m, 2H), 4.01 (m, 2H), 3.91 (t, J=7.3 Hz, 2H), 3.49 (s, 2H), 2.2 (t, J=7.3, 2H), 1.48 (m, 4H), 1.2 (m, 2H), 1.137 (t, J=0.134, 3H)

### Example 37

### 6-(7-chloro-2,3-dihydro-3-oxobenzo[b][1,4]thiazin-4-yl)-N-hydroxyhexanamide

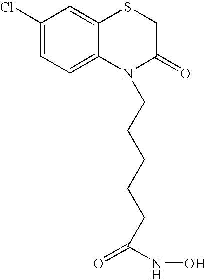

To a neat ethyl 6-(7-chloro-3-oxo-2,3-dihydrobenzo[b][1,4]thiazin-4-yl)hexanoate (500 mg, 1.45 mmol) N,O-Bis(trimethylsilyl)hydroxylamine (7.25 mmol, 1.3 g, 5 eq.) was added at room temperature. After stirring for 30 min a solution of 1N NaOH (2 ml) was added followed by the addition of methanol (˜7 ml). Then reaction mixture was concentrated via rotovap and then purified on silica gel column using methylene chloride/methanol as an eluting solvent (62 mg, 13%): ¹H NMR (300 MHz, d6-DMSO): δ 10.306 (s, 1H), 8.650 (s, 1H), 7.511 (m, 1H), 7.3 (m, 2H), 3.91 (t, J=7.3 Hz, 2H), 3.49 (s, 2H), 1.883 (t, J=7.3, 2H), 1.45 (m, 4H), 1.2 (m, 2H).

### Example 38

### In vitro receptor binding, enzyme and ADME-Tox assays of the compound of Example 20 (5-(5-cyano-1H-indol-1-yl)pentylboronic acid

This example shows the effects (5-(5-cyano-1H-indol-1-yl)pentylboronic acid in various in vitro receptor binding, enzyme and ADME-Tox assays. In each experiment, the respective reference compound was tested concurrently with 5-(5-cyano-1H-indol-1-yl)pentylboronic acid in order to assess the assay suitability. Reference compound were tested at several concentrations (for IC₅₀ or EC₅₀ value determination), and the data were compared with historical values previously determined.

Bind assay. The binding of (5-(5-cyano-1H-indol-1-yl)pentylboronic acid to the receptors was determined as described in Tables 1 and 2. The specific ligand binding to receptors is the difference between the total binding and the non-specific binding determined in the presence of an excess of unlabeled ligand. The results are expressed as the percent inhibition of control values percent in the presence of (5-(5-cyano-1H-indol-1-yl)pentylboronic acid as shown in Table 3. The mean values from two experiments, expressed as the percent of control binding was also determined (data not shown). The IC₅₀ values (concentration causing a half-maximal inhibition of control specific binding) and Hill coefficients (n_{H}) were determined by non-linear regression analysis of the competition curves using Hill equation curve fitting. The inhibition constants (Kᵢ) were calculated from the Cheng Prusoff equation (Kᵢ=IC₅₀/(1+(L/K_{D})), where L=concentration of radioligand in the assay, and K_{D}=affinity of the radioligand for the receptor) see Table 4

TABLE 1

| | | Reference | |
|---|---|---|---|
| Assay | Origin | Compound | Bibliography |
| A₁(h) | human | DPCPX | Townsend-Nicholson |
| | recombinant | | and Schofield (1994) |
| | (CHO cells) | | |
| A_{2A}(h) | human | NECA | Luthin et al. (1995) |
| | recombinant | | |
| | (HEK-293 | | |
| | cells) | | |
| A₃(h) | human | IB-MECA | Salvatore et al. (1993) |
| | recombinant | | |
| | (HEK-293 | | |
| | cells) | | |
| α₁ | rat cerebral | prazosin | Greengrass and |
| (non-selective) | cortex | | Bremner (1979) |
| α₂ | rat cerebral | yohimbine | Uhlen and Wikberg |
| (non-selective) | cortex | | (1991) |
| β₁(h) | human | atenolol | Levin et al. (2002) |
| | recombinant | | |
| | (HEK-293 | | |
| | cells) | | |
| β₂(h) | human | ICI 118551 | Smith and Teitler |
| | recombinant | | (1999) |
| | (Sf9 cells) | | |
| AT₁(h) | human | saralasin | Bergsma et al. (1992) |
| | recombinant | | |
| | (CHO cells) | | |
| AT₂(h) | human | saralasin | Tsuzuki et al. (1994) |
| | recombinant | | |
| | (Hela cells) | | |
| BZD (central) | rat cerebral | diazepam | Speth et al. (1979) |
| | cortex | | |
| B₁(h) | human | desArg¹⁰-KD | Jones et al. (1999) |
| | recombinant | | |
| | (CHO cells) | | |
| B₂(h) | human | NPC 567 | Pruneau et al. (1998) |
| | recombinant | | |
| | (CHO cells) | | |
| CB₁(h) | human | WIN 55212-2 | Matsuda et al. (1990) |
| | recombinant | | |
| | (HEK-293 | | |
| | cells) | | |
| CB₂(h) | human | WIN 55212-2 | Munro et al. (1993) |
| | recombinant | | |
| | (HEK-293 | | |
| | cells) | | |
| CCK_{A}(h) | human | CCK-8 | Bignon et al. (1999) |
| CCK₁ | recombinant | | |
| | (CHO cells) | | |
| CCK_{B}(h) | human | CCK-8 | Lee et al. (1993) |
| CCK₂ | recombinant | | |
| | (HEK-293 | | |
| | cells) | | |
| CRF₁ | rat pituitary | CRF | Okuyama et al. (1999) |
| | gland | | |
| D1(h) | human | SCH 23390 | Zhou et al. (1990) |
| | recombinant | | |
| | (CHO cells) | | |
| D2S(h) | human | (+)butaclamol | Grandy et al. (1989) |
| | recombinant | | |
| | (CHO cells) | | |
| D2(h) | human | (+)butaclamol | Mackenzie et al. |
| | recombinant | | (1994) |
| | (CHO cells) | | |
| D4.4(h) | human | clozapine | Van Tol et al. (1992) |
| | recombinant | | |
| | (CHO cells) | | |
| ET_{A}(h) | human | endothelin-1 | Buchan et al. (1994) |
| | recombinant | | |
| | (CHO cells) | | |
| ET_{B}(h) | human | endothelin-3 | Buchan et al. (1994) |
| | recombinant | | |
| | (CHO cells) | | |
| GABA | rat cerebral | GABA | Tsuji et al. (1988) |
| (non-selective) | cortex | | |
| AMPA | rat cereb-1 | L-glutamate | Murphy et al. (1987) |
| | cortex | | |
| Kainate | rat cerebral | kainic acid | Monaghan and |
| | cortex | | Cotman (1982) |
| NMDA | rat cerebral | CGS 19755 | Sills et al. (1991) |
| | cortex | | |
| H₁(h) | human | pyrilamine | Smit et al. (1996) |
| | recombinant | | |
| | (HEK-293 | | |
| | cells) | | |
| H₂(h) | human | cimetidine | Leurs et al. (1994) |
| | recombinant | | |
| | (CHO cells) | | |
| H₃ | rat cerebral | (R)α-Me-histamine | Arrang et al. (1990) |
| | cortex | | |
| I₁ | bovine adrenal | rilmenidine | Dontenwill et al. |
| (peripheral) | medulla glands | | (1999) |
| I₂ | rat cerebral | idazoxan | Brown et al. (1990) |
| (central) | cortex | | |
| LTD₄(h) | U-937 cells | LTD₄ | Frey et al. (1993) |
| MC₄(h) | human | NDP-α-MSH | Schioth et al. (1997) |
| | recombinant | | |
| | (HEK-293 | | |
| | cells) | | |
| M | rat cerebral | atropine | Richards (1990) |
| (non-selective) | cortex | | |
| NK₁(h) | U-373MG cells | [Sar⁹,Met(O₂)¹¹]-SP | Heuillet et al. (1993) |
| NK₂(h) | human | [N1e¹⁰]-NKA(4-10) | Aharony et al. (1993) |
| | recombinant | | |
| | (CHO cells) | | |
| NK₃(h) | human | SB 222200 | Suman-Chauhan et al. |
| | recombinant | | (1994) |
| | (CHO cells) | | |
| Y | rat cerebral | NPY | Goldstein et al. (1986) |
| (non-selective) | cortex | | |
| N (neuronal) | rat cerebral | nicotine | Pabreza et al. (1991) |
| (α-BGTX- | cortex | | |
| insensitive) | | | |
| Opiate | rat cerebral | naloxone | Childers et al. (1979) |
| (non-selective) | cortex | | |
| ORL1 (h) | human | nociceptin | Ardati et al. 1997) |
| (NOP) | recombinant | | |
| | (HEK-293 | | |
| | cells) | | |
| PCP | rat cerebral | MK 801 | Vignon et al. (1986) |
| | cortex | | |
| P2X | rat urinary | α,β-MeATP | Bo and Burnstock |
| | bladder | | (1990) |
| P2Y | rat cerebral | dATPαS | Simon et al. (1995) |
| | cortex | | |
| 5-HT | rat cerebral | serotonin | Peroutka and Snyder |
| (non-selective) | cortex | | (1979) |
| σ | rat cerebral | haloperidol | Shirayama et al. |
| (non-selective) | cortex | | (1993) |
| Glucocorticoid(h) | IM-9 cells | dexamethasone | Clark et al. (1996) |
| (GR) | (cytosol) | | |
| Estrogen(h) | MCF-7 cells | 17-β-estradiol | Sheen et al. (1985) |
| (ER) | (cytosol) | | |
| Progesterone(h) | MCF-7 cells | R 5020 | Eckert and |
| (PR) | (cytosol) | | Katzenellenbogen |
| | | | (1982) |
| Androgen(h) | LNCaP cells | methyltrienolone | Zava et al. (1979) |
| (AR) | (cytosol) | | |
| TRH | rat cerebral | TRH | Sharif and Burt (1983) |
| | cortex | | |
| V₁ₐ(h) | human | [d(CH₂)₁¹,Tyr(Me)₂]- | Tahara et al. (1998) |
| | recombinant | AVP | |
| | (CHO cells) | | |
| V₂(h) | human | AVP | Tahara et al. (1998) |
| | recombinant | | |
| | (CHO cells) | | |
| Ca²⁺ channel | rat cerebral | nitrendipine | Lee et al. (1984) |
| (L, DHP site) | cortex | | |
| Ca²⁺ channel | rat cerebral | diltiazem | Schoemaker and |
| (L, diltiazem site) | cortex | | Langer (1985) |
| (benzothiazepines) | | | |
| Ca²⁺ channel | rat cerebral | D 600 | Reynolds et al. (1986) |
| (L, verapamil site) | cortex | | |
| (phenylalkylamines) | | | |
| K⁺_{ATP} channel | rat cerebral | glibenclamide | Angel and Bidet 1991) |
| | cortex | | |
| K⁺_{V} channel | rat cerebral | α-dendrotoxin | Sorensen and |
| | cortex | | Blaustein (1989) |
| SK⁺_{Ca} channel | rat cerebral | apamin | Hugues et al. (1982) |
| | cortex | | |
| Na⁺ channel | rat cerebral | veratridine | Brown (1986) |
| (site 2) | cortex | | |
| Cl-channel | rat cerebral | picrotoxinin | Lewin et al. (1989) |
| | cortex | | |
| NE transporter(h) | human | protriptyline | Pacholczyk et al. |
| | recombinant | | (1991) |
| | (MDCK cells) | | |
| DA transporter(h) | human | BTCP | Pristupa et al. (1994) |
| | recombinant | | |
| | (CHO cells) | | |
| GABA transporter | rat cerebral | nipecotic acid | Shank et al. (1990) |
| | cortex | | |
| Choline transporter | rat striatum | hemicholinium-3 | Vickroy et al. (1984) |
| 5-HT transporter(h) | human | imipramine | Tatsumi et al. (1999) |
| | recombinant | | |
| | (HEK-293 cells) | | |

TABLE 2

| | | | | | | Method of |
|---|---|---|---|---|---|---|
| Assay | Ligand | Conc. | | Non Specific | Incubation | Detection |
| A₁(h) | [³H]DPCPX | 1 | nM | DPCPX | 60 min./ | Scintillation |
| | | | | (1 μM) | 22° C. | counting |
| A_{2A}(h) | [³H]CGS 21680 | 6 | nM | NECA | 90 min./ | Scintillation |
| | | | | (10 μM) | 22° C. | counting |
| A₃(h) | [¹²⁵I]AB-MECA | 0.1 | nM | IB-MECA | 90 min./ | Scintillation |
| | | | | (1 μM) | 22° C. | counting |
| α₁ | [³H]prazosin | 0.25 | nM | prazosin | 60 min./ | Scintillation |
| (non- | | | | (0.5 μM) | 22° C. | counting |
| selective) | | | | | | |
| α₂ | [³H]RX 821002 | 0.5 | nM | (−)epinephrine | 30 min./ | Scintillation |
| (non- | | | | (100 μM) | 22° C. | counting |
| selective) | | | | | | |
| β₁(h) | [³H](−)CGP 12177 | 0.15 | nM | alprenolol | 60 min./ | Scintillation |
| | | | | (50 μM) | 22° C. | counting |
| β₂(h) | [³H](−)CGP 12177 | 0.15 | nM | alprenolol | 60 min./ | Scintillation |
| | | | | (50 μM) | 22° C. | counting |
| AT₁(h) | [¹²⁵I][Sar¹,Iie⁸]-AT II | 0.05 | nM | angiotensin II | 60 min./ | Scintillation |
| | | | | (10 μM) | 37° C. | counting |
| AT₂(h) | [¹²⁵I]CGP 42112A | 0.05 | nM | angiotensin II | 180 min./ | Scintillation |
| | | | | (1 μM} | 37° C. | counting |
| BZD | [³H]flunitrazepam | 0.4 | nM | diazepam | 60 min./ | Scintillation |
| (central) | | | | (3 μM) | 4° C. | counting |
| B₁(h) | [³H]desArg¹⁰-KD | 0.35 | nM | desArg⁹[Leu⁸]- | 60 min./ | Scintillation |
| | | | | BK | 22° C. | counting |
| | | | | (10 μM) | | |
| B₂(h) | [³H]bradykinin | 0.2 | nM | bradykinin | 45 min./ | Scintillation |
| | | | | (1 μM) | 22° C. | counting |
| CB₁(h) | [³H]WIN 55212-2 | 2 | nM | WIN 55212-2 | 90 min./ | Scintillation |
| | | | | (10 μM) | 37° C. | counting |
| CB₂(h) | [³H]WIN 55212-2 | 0.8 | nM | WIN 55212-2 | 90 min./ | Scintillation |
| | | | | (5 μM) | 30° C. | counting |
| CCK_{A}(h) | [¹²⁵I]CCK-8 | 0.08 | nM | CCK-8 | 60 min./ | Scintillation |
| (CCK₁) | | | | (1 μM) | 22° C. | counting |
| CCK_{B}(h) | [¹²⁵I]CCK-8 | 0.025 | nM | CCK-8 | 60 min./ | Scintillation |
| (CCK₂) | | | | (1 μM) | 22° C. | counting |
| CRF₁ | [¹²⁵I]Tyr⁰-CRF | 0.1 | nM | CRF | 120 min./ | Scintillation |
| | | | | (1 μM) | 22° C. | counting |
| D1(h) | [³H]SCH 23390 | 0.3 | nM | SCH 23390 | 60 min./ | Scintillation |
| | | | | (1 μM) | 22° C. | counting |
| D2S(h) | [³H]spiperone | 0.3 | nM | (+)butaclamol | 60 min./ | Scintillation |
| | | | | (10 μM) | 22° C. | counting |
| D3(h) | [³H]spiperone | 0.3 | nM | (+)butaclamol | 60 min./ | Scintillation |
| | | | | (10 μM) | 22° C. | counting |
| D4.4(h) | [³H]spiperone | 0.3 | nM | (+)butaclamol | 60 min./ | Scintillation |
| | | | | (10 μM) | 22° C. | counting |
| ET_{A}(h) | [¹²⁵I]endothelin-1 | 0.03 | nM | endothelin-1 | 120 min./ | Scintillation |
| | | | | (0.1 μM) | 37° C. | counting |
| ET_{B}(h) | [¹²⁵I]endothelin-1 | 0.03 | nM | endothelin-1 | 120 min./ | Scintillation |
| | | | | (0.1 μM) | 37° C. | counting |
| GABA | [³H]GABA | 10 | nM | GABA | 20 min./ | Scintillation |
| (non- | | | | (100 μM) | 22° C. | counting |
| selective) | | | | | | |
| AMPA | [H]AMPA | 8 | nM | L-glutamate | 60 min./ | Scintillation |
| | | | | (1 mM) | 4° C. | counting |
| Kainate | [³H]kainic acid | 5 | nM | L-glutamate | 60 min./ | Scintillation |
| | | | | (1 mM) | 4° C. | counting |
| NMDA | [³H]CGP 39653 | 5 | nM | L-glutamate | 60 min./ | Scintillation |
| | | | | (100 μM) | 4° C. | counting |
| H₁(h) | [³H]pyrilamine | 3 | nM | pyrilamine | 60 min./ | Scintillation |
| | | | | (1 μM) | 22° C. | counting |
| H₂(h) | [¹²⁵I]APT | 0.2 | nM | tiotidine | 120 min./ | Scintillation |
| | | | | (100 μM) | 22° C. | counting |
| H₃ | [³H](R)α-Me- | 1 | nM | (R)α-Me- | 120 min./ | Scintillation |
| | histamine | | | histamine | 22° C. | counting |
| | | | | (5 μM) | | |
| I₁ | [³H]clonidine | 15 | nM | rilmenidine | 30 min./ | Scintillation |
| (peripheral) | (+10 μM RX821002) | | | (10 μM) | 22° C. | counting |
| I₂ | [³H]idazoxan | 2 | nM | cirazoline | 30 min./ | Scintillation |
| (central) | | | | (10 μM) | 22° C. | counting |
| LTD₄(h) | [³H]LTD₄ | 0.3 | nM | LTD₄ | 60 min./ | Scintillation |
| | | | | (1 μM) | 22° C. | counting |
| MC₄(h) | [¹²⁵I]NDP-α-MSH | 0.05 | nM | NDP-α-MSH | 60 min./ | Scintillation |
| | | | | (1 μM) | 37° C. | counting |
| M | [³H]QNB | 0.05 | nM | atropine | 120 min./ | Scintillation |
| (non- | | | | (1 μM) | 22° C. | counting |
| selective) | | | | | | |
| NK₁(h) | [¹²⁵I][Sar⁹,Met(O₂)¹¹]- | 0.15 | nM | [Sar⁹,Met(O₂)¹¹]- | 60 min./ | Scintillation |
| | SP | | | SP | 22° C. | counting |
| | | | | (1 μM) | | |
| NK₂(h) | [¹²⁵I]NKA | 0.1 | nM | [Nle¹⁰]-NKA | 90 min./ | Scintillation |
| | | | | (4-10) | 22° C. | counting |
| | | | | (10 μM) | | |
| NK₃(h) | [³H]SR 142801 | 0.2 | nM | SB 222200 | 90 min./ | Scintillation |
| | | | | (10 μM) | 22° C. | counting |
| Y | [³H]NPY | 0.5 | nM | NPY | 90 min./ | Scintillation |
| (non- | | | | (1 μM) | 22° C. | counting |
| selective) | | | | | | |
| N | [³H]cytosine | 1.5 | nM | nicotine | 75 min./ | Scintillation |
| (neuronal) | | | | (10 μM) | 4° C. | counting |
| (α-BGTX- | | | | | | |
| insensitive) | | | | | | |
| Opiate | [³H]naloxone | 1 | nM | naloxone | 40 | Scintillation |
| (non- | | | | (1 μM) | inin./22° C. | counting |
| selective) | | | | | | |
| ORL1(h) | [³H]nociceptin | 0.2 | nM | nociceptin | 60 min./ | Scintillation |
| (NOP) | | | | (1 μM) | 22° C. | counting |
| PCP | [³H]TCP | 5 | nM | MK 801 | 45 min./ | Scintillation |
| | | | | (10 μM) | 22° C. | counting |
| P2X | [³H]α,β-MeATP | 3 | nM | α,β-MeATP | 120 min./ | Scintillation |
| | | | | (10 μM) | 4° C. | counting |
| P2Y | [³⁵S]dATPαS | 10 | nM | dATPαS | 60 min./ | Scintillation |
| | | | | (10 μM) | 22° C. | counting |
| 5-HT | [³H]serotonin | 2 | nM | serotonin | 15 min./ | Scintillation |
| (non- | | | | (10 μM) | 37° C. | counting |
| selective) | | | | | | |
| σ | [³H]DTG | 8 | nM | haloperidol | 120 min./ | Scintillation |
| (non- | | | | (10 μM) | 22° C. | counting |
| selective) | | | | | | |
| Glucocorticoid | [³H]dexamethasone | 1.5 | nM | triamcinolone | 18 h./4° C. | Scintillation |
| (h) | | | | (10 μM) | | counting |
| (GR) | | | | | | |
| Estrogen(h) | [³H]estradiol | 1 | TIM | 17-(3- | 20 h./4° C. | Scintillation |
| (ER) | | | | estradiol | | counting |
| | | | | (6 μM) | | |
| Progesterone | [³H]R 5020 | 2 | nM | R 5020 | 20 h./4° C. | Scintillation |
| (h) | | | | (1 μM) | | counting |
| (PR) | | | | | | |
| Androgen | [³H]methyltrienolone | 0.5 | nM | mibolerone | 24 h./4° C. | Scintillation |
| (h) | | | | (1 μM) | | counting |
| (AR) | | | | | | |
| TRH | [³H]Me-TRH | 2 | nM | TRH | 6 h./4° C. | Scintillation |
| | | | | (30 μM) | | counting |
| V₁ₐ(h) | [³H]AVP | 0.3 | nM | AVP | 60 min./ | Scintillation |
| | | | | (1 μM) | 22° C. | counting |
| V₂(h) | [³H]AVP | 0.3 | nM | AVP | 90 min./ | Scintillation |
| | | | | (1 μM) | 22° C. | counting |
| Ca²⁺ | [³H](+)PN 200-110 | 0.04 | nM | nifedipine | 90 min./ | Scintillation |
| channel | | | | (1 μM) | 22° C. | counting |
| (L, DHP | | | | | | |
| site) | | | | | | |
| Ca²⁺ | [³H]diltiazern | 5 | nM | diltiazem | 120 min./ | Scintillation |
| channel | | | | (10 μM) | 22° C. | counting |
| (L, | | | | | | |
| diltiazem | | | | | | |
| site) | | | | | | |
| (benzothiazepines) | | | | | | |
| Ca²⁺ | [³H](−)D 888 | 0.5 | nM | D 600 | 60 min./ | Scintillation |
| channel | | | | (10 μM) | 22° C. | counting |
| (L, | | | | | | |
| verapamil | | | | | | |
| site) | | | | | | |
| (phenylalkylamines) | | | | | | |
| K⁺_{ATP} | [³H]glibenclamide | 0.1 | nM | glibenclamide | 60 min./ | Scintillation |
| channel | | | | (1 μM) | 22° C. | counting |
| K⁺_{V} | [¹²⁵I]α-dendrotoxin | 0.01 | nM | α-dendrotoxin | 30 min./ | Scintillation |
| channel | | | | (50 nM) | 22° C. | counting |
| SK⁺_{CA} | [¹²⁵I]apamin | 0.004 | nM | apamin | 30 min./ | Scintillation |
| channel | | | | (0.1 μM) | 0° C. | counting |
| Na⁺ | [³H]batrachotoxinin | 10 | nM | veratridine | 60 min./ | Scintillation |
| channel | | | | (300 μM) | 22° C. | counting |
| (site 2) | | | | | | |
| C1 channel | [³⁵S]TBPS | 3 | nM | picrotoxinin | 90 min./ | Scintillation |
| | | | | (20 μM) | 22° C. | counting |
| NE | [³H]nisoxetine | 1 | nM | desipramine | 60 min./ | Scintillation |
| transporter | | | | (1 μM) | 4° C. | counting |
| (h) | | | | | | |
| DA | [³H]GBR12935 | 0.5 | nM | BTCP | 120 min./ | Scintillation |
| transporter | | | | (10 μM) | 4° C. | counting |
| (h) | | | | | | |
| GABA | [³H]GABA | 10 | nM | GABA | 30 min./ | Scintillation |
| transporter | (+10 μM | | | (1 mM) | 22° C. | counting |
| | isogavacine) | | | | | |
| | (+10 μM baclofen) | | | | | |
| Choline | [³H]hemicholinium-3 | 3 | nM | hemicholinium-3 | 30 min./ | Scintillation |
| transporter | | | | (10 μM) | 22° C. | counting |
| 5-HT | [³H]imipramine | 2 | nM | imipramine | 30 min./ | Scintillation |
| transporter | | | | (10 μM) | 22° C. | counting |
| (h) | | | | | | |

TABLE 3

| | Test | % Inhibition |
|---|---|---|
| | Concentration | of Control |
| Assay | (M) | Specific Binding |
| A₁(h) | 1.0E−05 | 7 |
| A_{2A}(h) | 1.0E−05 | 16 |
| A₃(h) | 1.0E−05 | −12 |
| α₁ (non-selective) | 1.0E−05 | 21 |
| α₁ (non-selective) | 1.0E−05 | 7 |
| β₁(h) | 1.0E−05 | 7 |
| β₁(h) | 1.0E−05 | 5 |
| AT₁(h) | 1.0E−05 | −3 |
| AT₂(h) | 1.0E−05 | 3 |
| BZD (central) | 1.0E−05 | 18 |
| B₁(h) | 1.0E−05 | −5 |
| B₂(h) | 1.0E−05 | 18 |
| CB₁(h) | 1.0E−05 | −2 |
| CB₂(h) | 1.0E−05 | −12 |
| CCK_{A}(h) (CCK₁) | 1.0E−05 | 7 |
| CCK_{B}(h) (CCK₂) | 1.0E−05 | 5 |
| CRF₁ | 1.0E−05 | −23 |
| D1(h) | 1.0E−05 | 0 |
| D2S(h) | 1.0E−05 | 23 |
| D3(h) | 1.0E−05 | 4 |
| D4.4(h) | 1.0E−05 | 1 |
| 937033-1 | | |
| ET_{A}(h) | 1.0E−05 | −25 |
| 937033-1 | | |
| ET_{B}(h) | 1.0E−05 | 10 |
| 937033-1 | | |
| GABA (non-selective) | 1.0E−05 | −15 |
| 937033-1 | | |
| AMPA | 1.0E−05 | −3 |
| Kainate | 1.0E−05 | −6 |
| NMDA | 1.0E−05 | 19 |
| H₁(h) | 1.0E−05 | 6 |
| H₂(h) | 1.0E−05 | 15 |
| H₃ | 1.0E−05 | 0 |
| I₁ (peripheral) | 1.0E−05 | −6 |
| I₂ (central) | 1.0E−05 | 1 |
| LTD₄(h) | 1.0E−05 | 12 |
| MC₄ (h) | 1.0E−05 | 2 |
| M (non-selective) | 1.0E−05 | −4 |
| NK₁(h) | 1.0E−05 | 12 |
| NK₂(h) | 1.0E−05 | −2 |
| NK₃(h) | 1.0E−05 | 3 |
| Y (non-selective) | 1.0E−05 | 14 |
| N (neuronal) (α-BGTX-insensitive) | 1.0E−05 | 12 |
| Opiate (non-selective) | 1.0E−05 | 20 |
| ORL1(h) (NOP) | 1.0E−05 | 0 |
| PCP | 1.0E−05 | 5 |
| P2X | 1.0E−05 | 14 |
| P2Y | 1.0E−05 | −19 |
| 5-HT (non-selective) | 1.0E−05 | −1 |
| cs (non-selective) | 1.0E−05 | 5 |
| Glucocorticoid(h) (GR) | 1.0E−05 | −7 |
| Estrogen(h) (ER) | 1.0E−05 | 3 |
| Progesterone(h) (PR) | 1.0E−05 | 79 |
| Androgen(h) (AR) | 1.0E−05 | 40 |
| TRH | 1.0E−05 | −17 |
| V₁ₐ(h) | 1.0E−05 | 24 |
| V₂(h) | 1.0E−05 | 5 |
| Ca²⁺channel (L, DHP site) | 1.0E−05 | −10 |
| Ca²⁺channel (L, diltiazem site) | 1.0E−05 | −1 |
| (benzothiazepines) | | |
| Ca²⁺ channel (L, verapamil site) | 1.0E−05 | −12 |
| (phenylalkylamines) | | |
| K⁺_{ATP} channel | 1.0E−05 | 25 |
| K⁺ᵥ channel | 1.0E−05 | −5 |
| SK⁺_{ca} channel | 1.0E−05 | −15 |
| Na⁺ channel (site 2) | 1.0E−05 | 13 |
| 937033-1 | | |
| C1 channel | 1.0E−05 | 26 |
| NE transporter(h) | 1.0E−05 | 15 |
| DA transporter(h) | 1.0E−05 | 47 |
| GABA transporter | 1.0E−05 | −13 |
| Choline transporter | 1.0E−05 | 1 |
| 937033-1 | | |
| 5-HT transporter(h) | 1.0E−05 | 90 |

TABLE 4

| | IC₅₀ | Kᵢ | |
|---|---|---|---|
| Assay/Reference Compound | (M) | (M) | n_{H} |
| A₁(h)/DPCPX | 3.4E−08 | 2.1E−08 | 1.0 |
| A_{2A}(h) NECA | 3.7E−08 | 3.0E−08 | 1.0 |
| A₃₍ₕ₎ IB-MECA | 5.2E−09 | 3.6E−09 | 0.9 |
| α₁(non-selective)/prazosin | 3.4E−09 | 9.0E−10 | 1.6 |
| α₂ (non-selective)/yohimbine | 6.8E−08 | 2.9E−08 | 1.1 |
| β₁(h)/atenolol | 1.7E−07 | 1.2E−07 | 0.7 |
| β₂(h)/ICI 118551 | 1.9E−09 | 8.4E−10 | 1.3 |
| AT₁(h)/saralasin | 1.8E−09 | 1.3E−09 | 1.1 |
| AT₂(h)/saralasin | 2.4E−10 | 9.1E−11 | 0.8 |
| BZD (central)/diazepam | 2.2E−08 | 1.9E−08 | 1.2 |
| B₁(h)/desArg10-KD | 6.7E−10 | 1.3E−10 | 1.1 |
| B₂(h)/NPC 567 | 2.2E−08 | 1.4E−08 | 0.6 |
| CB₁(h)/WIN 55212-2 | 2.0E−08 | 1.5E−08 | 1.9 |
| CB₂(h)/WIN 55212-2 | 4.3E−09 | 1.5E−09 | 0.7 |
| CCK_{A}(h) (CCKI)/CCK-8 | 5.5E−10 | 4.1E−10 | 1.2 |
| CCK_{B}(h) (CCK2)/CCK-8 | 4.0E−09 | 2.6E−09 | 1.3 |
| CRF₁/CRF | 6.8E−09 | 2.8E−09 | 0.6 |
| D1(h)/SCH 23390 | 3.2E−10 | 1.3E−10 | 1.2 |
| D2S(h)/(+)butaclamol | 1.4E−08 | 5.0E−09 | 1.2 |
| D3(h)/(+)butaclamol | 2.7E−08 | 6.0E−09 | 1.0 |
| D4.4 (1)/clozapine | 8.4E−08 | 3.6E−08 | 1.2 |
| ET_{A}(h)/endothelia-1 | 2.4E−10 | 2.3E−10 | 1.1 |
| ET_{B}(h)/endothelia-3 | 2.3E−10 | 2.0E−10 | 2.4 |
| GAGA (non-selective)/GAGA | 3.9E−08 | 2.4E−08 | 1.3 |
| AMPA/L-glutamate | 1.3E−06 | 1.2E−06 | 1.4 |
| Kainate/kainic acid | 3.9E−08 | 3.1E−08 | 0.8 |
| NMDA/CGS 19755 | 1.6E−06 | 1.3E−06 | 0.7 |
| H₁(h)/pyrilamine | 4.9E−09 | 1.8E−09 | 1.1 |
| H₂(h) | 3.6E−07 | 3.4E−07 | 0.9 |
| cimetidine | | | |
| H₃ | 2.9E−09 | 1.2E−09 | 1.0 |
| (R)a-Me-histamine | | | |
| I₁ (peripheral) | 1.9E−07 | 9.6E−08 | 0.9 |
| rilmenidine | | | |
| I₂ (central) | 7.4E−09 | 4.9E−09 | 0.9 |
| idazoxan | | | |
| LTD₄(h) | 1.3E−09 | 8.7E−10 | 0.7 |
| LTD₄ | | | |
| MC₄(h)/NDP-a-MSH | 2.5E−10 | 2.0E−10 | 0.9 |
| M (non-selective)/atropine | 6.1E−10 | 1.0E−10 | 1.2 |
| NK₁(h)/[Sar⁹,Met(O₂)¹¹]-SP | 3.9E−10 | 1.8E−10 | 0.9 |
| NK₂(h)/N1e¹⁰]-NKA(4-10) | 6.0E−09 | 3.2E−09 | 0.7 |
| NK₃(h)/SB 222200 | 6.5E−08 | 3.3E−08 | 1.1 |
| Y (non-selective)/NPY | 9.9E−10 | 7.6E−10 | 1.3 |
| N (neuronal) (α-BGTX-insensitive)/nicotine | 8.9E−09 | 4.9E−09 | 1.1 |
| Opiate (non-selective)/naloxone | 1.1E−09 | 7.6E−10 | 1.3 |
| ORL1(h) (NOP)/nociceptin | 5.1E−09 | 2.3E−09 | 2.7 |
| PCP/MK 801 | 3.0E−09 | 2.8E−09 | 0.8 |
| P2X/α,β-MeATP | 8.1E−09 | 3.8E−09 | 0.6 |
| P2Y/dATPαS | 1.0E−07 | 5.2E−08 | 1.3 |
| 5-HT (non-selective)/serotonin | 2.2E−09 | 1.2E−09 | 0.9 |
| σ (non-selective)/haloperidol | 4.6E−08 | 3.6E−08 | 0.6 |
| Glucocorticoid(h) (GR)/dexamethasone | 2.6E−09 | 1.3E−09 | 1.1 |
| Estrogen(h) (ER)/17-β-estradiol | 9.1E−10 | 5.9E−11 | 1.0 |
| Progesterone(h) (PR)/R 5020 | 8.9E−09 | 3.0E−09 | 1.1 |
| Androgen(h) (AR)/methyltrienolone | 4.5E−09 | 3.6E−09 | 1.5 |
| TRH/TRH | 4.9E−08 | 3.0E−08 | 0.9 |
| V₁ₐ(h)/[d(CH₂)₅¹,Tyr(Me)₂]-AVP | 3.4E−09 | 2.1E−09 | 1.5 |
| V₂(h)/AVP | 1.1E−09 | 6.2E−10 | 0.9 |
| Ca²⁺ channel (L, DHP site)/nitrendipine | 9.3E−10 | 3.1E−10 | 1.5 |
| Ca²⁺ channel (L, diltiazem site) | 3.3E−08 | 3.0E−08 | 0.9 |
| (benzothiazepines)/diltiazem | | | |
| Ca²⁺ channel (L, verapamil site) | 5.OE−09 | 8.4E−10 | 0.6 |
| (phenylalkylamines)/D 600 | | | |
| K⁺_{ATP} channel/glibenclamide | 2.5E−09 | 8.3E−10 | 1.4 |
| K⁺ᵥ channel/α-dendrotoxin | 1.3E−09 | 1.1E−09 | 3.1 |
| SK⁺_{ca} channel/apamin | 1.9E−11 | 1.2E−11 | 1.2 |
| Na⁺ channel (site 2)/veratridine | 4.6E−06 | 4.1E−06 | 1.1 |
| C1 channel/picrotoxinin | 3.5E−07 | 2.9E−07 | 0.9 |
| NE transporter(h)/protriptyline | 1.4E−08 | 1.1E−08 | 1.7 |
| DA transporter(h)/BTCP | 1.7E−08 | 1.0E−08 | 0.5 |
| GABA transporter/nipecotic acid | 3.4E−06 | 3.4E−06 | 0.9 |
| Choline transporter/hemicholinium-3 | 8.0E−09 | 5.5E−09 | 0.7 |
| 5-HT transporter(h)/imipramine | 1.2E−08 | 7.2E−09 | 1.0 |

Enzyme assays. The effect of (5-(5-cyano-1H-indol-1-yl)pentylboronic acid on the enzymes of Table 5 was determined with the using the experimental conditions described in Table 6.

TABLE 5

| | | Reference | |
|---|---|---|---|
| Assay | Origin | Compound | Bibliography |
| Phosphodiesterase 1 | bovine brain | 8-methoxy-IBMX | Nicholson et al. |
| | | | (1989) |
| Phosphodiesterase 2(h) | differentiated U- | EHNA | Torphy et al. (1992) |
| | 937 cells | | |
| Phosphodiesterase 3(h) | human platelets | milrinone | Weishaar et al. |
| | | | (1986) |
| Phosphodiesterase 4(h) | U-937 cells | rolipram | Torphy et al. (1992) |
| Phosphodiesterase 5(h) | human platelets | dipyridamole | Weishaar et al. |
| | | | (1986) |
| Adenylyl cyclase | rat brain | forskolin | Salomon et al. |
| (basal) | | | (1974) |
| Guanylyl cyclase | bovine lung | sodium | Wolin et al. (1982) |
| (basal) | | nitroprusside | |
| Protein kinase C | rat brain | staurosporine | Hannun et al. (1985) |
| Acetylcholinesterase(h) | human | neostigmine | Ellman et al. (1961) |
| | recombinant | | |
| | (HEK-293 cells) | | |
| Catechol- | porcine liver | Ro 41-0960 | Muller-Enoch et al. |
| O-methyl transferase | | | (1976) |
| GABA transaminase | rat brain | AoAA | Losher (1981) |
| MAO-A(h) | human placenta | clorgyline | Weyler and Salach |
| | | | (1985) |
| MAO-B(h) | human platelets | deprenyl | Uebelhack et al. |
| | | | (1998) |
| Phenylethanolamine- | bovine adrenal | LY 78335 | Betito et al. (1993) |
| N-methyl transferase | medulla | | |
| Tyrosine hydroxylase | rat striatum | 3-iodo L-tyrosine | Nagatsu et al. (1964) |
| ATPase (Na⁺/K⁺) | dog kidney | ouabain | Fiske and Subbarow |
| | | | (1925) |

TABLE 6

| | Substrate/Stimulus/ | | Reaction | Method of |
|---|---|---|---|---|
| Assay | Tracer | Incubation | Product | Detection |
| Phosphodiesterase 1 | [³H]cAMP + | 30 min./30° C. | [³H]5′AMP | Scintillation |
| | cAMP (1 μM) | | | counting |
| Phosphodiesterase 2(h) | [³H]cAMP + | 30 min./30° C. | [³H]5′AMP | Scintillation |
| | cAMP (1 | | | counting |
| | .tM) | | | |
| Phosphodiesterase 3(h) | [³H]cAMP + | 30 min./30° C. | [³H]5′AMP | Scintillation |
| | cAMP (0.1 μM) | | | counting |
| Phosphodiesterase 4(h) | [³H]cAMP + | 30 min./30° C. | [³H]5′AMP | Scintillation |
| | cAMP (1 μM) | | | counting |
| Phosphodiesterase 5(h) | [³H]cGMP + | 30 min./30° C. | [³H]5′GMP | Scintillation |
| | cGMP (1 μM) | | | counting |
| Adenylyl cyclase | ATP | 30 min./30° C. | cAMP | RIA |
| (basal) | (0.5 mM) | | | |
| Guanylyl cyclase | GTP | 15 min./30° C. | cGMP | RIA |
| (basal) | (0.1 mm) | | | |
| Protein kinase C | [γ³³P]ATP + | 20 min./30° C. | [γ³³P]histone H₁ | Scintillation |
| | histone H₁ | | | counting |
| | (200 μg/ml) | | | |
| Acetylcholinesterase(h) | AMTCh | 30 min./37° C. | thio-conjugate | Photometry |
| | (50 μM) | | | |
| Catechol- | esculetin | 30 min./37° C. | scopoletin | Fluorimetry |
| O-methyl transferase | (1 μM) | | | |
| GABA transaminase | GABA (9 mM) + | 60 min./37° C. | succinic | Fluorimetry |
| | α- | | semialdehyde | |
| | ketoglutarate (9 mM) | | | |
| MAO-A(h) | kynuramine | 30 min./30° C. | 4-Ohquinoline | Photometry |
| | (0.15 mM) | | | |
| MAO-B(h) | benzylamine | 45 min./37° C. | benzaldehyde | Photometry |
| | (0.5 mM) | | | |
| Phenylethanolamine- | [¹⁴C]SAM | 20 min./37° C. | [¹⁴C]metanephrine | Scintillation |
| N-methyl transferase | (4 μM) + | | | counting |
| | normetanephrine | | | |
| | (28 mM) | | | |
| Tyrosine hydroxylase | [³H]tyrosine | 40 min./37° C. | [³H]H₂O | Scintillation |
| | (10 μM) | | | counting |
| ATPase (Na⁺/K⁺) | ATP | 60 min./37° C. | Pi | Photometry |
| | (2 mM) | | | |

Enzyme Results. The mean values for the inhibitory effects of (5-(5-cyano-1H-indol-1-yl)pentylboronic acid on the assayed enzymes is summarized in Table 7. The IC₅₀ value for each reference compound is indicated in Table 8. Each is within accepted limits of the historic average ±0.5 log units. The mean values for the stimulatory effects of (5-(5-cyano-1H-indol-1-yl)pentylboronic acid summarized Table 9. The EC₅₀ value for each reference compound is indicated in Table 10. Each is within accepted limits of the historic average ±0.5 tog units.

TABLE 7

| | Test | |
|---|---|---|
| | Concentration | % Inhibition of |
| Assay | (M) | Control Values |
| Phosphodiesterase 1 | 1.0E−05 | 7 |
| Phosphodiesterase 2(h) | 1.0E−05 | 95 |
| Phosphodiesterase 3(h) | 1.0E−05 | 95 |
| Phosphodiesterase 4(h) | 1.0E−05 | 91 |
| Phosphodiesterase 5(h) | 1.0E−05 | 94 |
| Protein kinase C | 1.0E−05 | 0 |
| Acetylcholinesterase(h) | 1.0E−05 | 11 |
| Catechol-0-methyl | 1.0E−05 | −5 |
| transferase | | |
| GABA transaminase | 1.0E−05 | −2 |
| MAO-A(h) | 1.0E−05 | 6 |
| MAO-B(h) | 1.0E−05 | −8 |
| Phenylethanolamine- | 1.0E−05 | 1 |
| N-methyl transferase | | |
| Tyrosine hydroxylase | 1.0E−05 | 4 |
| ATPase (Na⁺/K⁺) | 1.0E−05 | 8 |

TABLE 8

| Assay | IC₅₀ | |
|---|---|---|
| Reference Compound | (M) | n_{H} |
| Phosphodiesterase 1 | 2.4E−06 | 0.5 |
| 8-methoxy-IBMX | | |
| Phosphodiesterase 2(h) | 4.1E−06 | 0.4 |
| EHNA | | |
| Phosphodiesterase 3(h) | 2.7E−07 | 0.8 |
| milrinone | | |
| Phosphodiesterase 4(h) | 7.1E−07 | 0.9 |
| rolipram | | |
| Phosphodiesterase 5(h) | 2.0E−06 | 1.5 |
| dipyridamole | | |
| Protein kinase C | 9.2E−08 | 1.2 |
| staurosporine | | |
| Acetylcholinesterase(h) | 3.3E−08 | 1.5 |
| neostigmine | | |
| Catechol-O-methyl transferase | 5.1E−08 | 1.7 |
| Ro 41-0960 | | |
| GABA transaminase | 2.1E−07 | 1.1 |
| AoAA | | |
| MAO-A(h) | 4.2E−08 | 1.1 |
| clorgyline | | |
| MAO-B(h) | 8.9E−08 | 0.7 |
| deprenyl | | |
| Phenylethanolamine-N-methyl transferase | 3.9E−05 | 1.4 |
| LY 78335 | | |
| Tyrosine hydroxylase | 9.9E−07 | 1.1 |
| 3-iodo L-tyrosine | | |
| ATPase (Na⁺/K⁺) | 9.2E−07 | 1.2 |
| ouabain | | |

TABLE 9

| | Test | % Stimulation |
|---|---|---|
| Assay | Concentration (M) | Relative to Control |
| Adenylyl cyclase | 1.0E−05 | −3 |
| (basal) | | |
| Guanylyl cyclase | 1.0E−05 | 0 |
| (basal) | | |

TABLE 10

| | EC₅₀ | |
|---|---|---|
| Assay/Reference Compound | (M) | n_{H} |
| Adenylyl cyclase (basal)/forskolin | 1.1E−05 | 1.0 |
| Guanylyl cyclase (basal)/sodium nitroprusside | 4.2E−06 | 0.6 |

ADME-Tox: In vitro Metabolism. The ADME-Toxicology in vitro metabolism of (5-(5-cyano-1H-indol-1-yl)pentylboronic acid was determined using the procedures cited in Table 11. The mean values from two experiments of the effects of 1.0E-05(M) (5-(5-cyano-1H-indol-1-yl)pentylboronic acid on receptors is summarized in Table 4.

TABLE 11

| | | Reference | |
|---|---|---|---|
| Assay | Source | Compound | Bibliography |
| CYPIA2 Inhibition | Human | furafylline | Crespi et al. |
| (CEC substrate) | recombinant | | (1997) |
| | (1.25 pmol/mL) | | |
| CYP2C9 Inhibition | Human | sulfaphenazole | Crespi et al. |
| (7-MFC substrate) | recombinant | | (1997) |
| | (15 pmol/mL) | | |
| CYP2C19 | Human | tranylcypromine | Ono et al. |
| Inhibition | recombinant | | (1996) |
| (CEC substrate) | (10 pmol/mL) | | |
| CYP2D6 Inhibition | Human | quinidine | Ono et al. |
| (7-MFC substrate) | recombinant | | (1996) |
| | (50 pmol/mL) | | |
| CYP3A4 Inhibition | Human | ketoconazole | Stresser et |
| (BFC substrate) | recombinant | | al. (2000) |
| | (2.5 pmol/mL) | | |

TABLE 12

| | | | Detected | Analytical |
|---|---|---|---|---|
| Assay | Substrate Cofactor | Incubation | Component | Method |
| CYP1A2 | CEC (5 μM), | 0 and 30 min, | CHC | Fluorimetry |
| Inhibition | NADP (1.3 mM), | 37° C. | | |
| (CEC substrate) | G6P (3.3 mM), | | | |
| | G6PDHase (0.4 U/mL) | | | |
| CYP2C9 | MFC (50 μM), | 0 and 80 min, | HFC | Fluorimetry |
| Inhibition | NADP (1.3 mM), | 37° C. | | |
| (7-MFC substrate) | G6P (3.3 mM), | | | |
| | G6PDHase (0.4 U/mL) | | | |
| CYP2C 19 | CEC (25 μM), | 0 and 60 min, | CHC | Fluorimetry |
| Inhibition | NADP (1.3 mM), | 37° C. | | |
| (CEC substrate) | G6P (3.3 mM), | | | |
| | G6PDHase (0.4 U/mL) | | | |
| CYP2D6 | 7-MFC (50 μM), | 0 and 60 min., | HFC | Fluorimetry |
| Inhibition | NADP (1.3 mM), | 37° C. | | |
| (7-MFC substrate) | G6P (3.3 mM), | | | |
| | G6PDHase (0.4 U/mL) | | | |
| CYP3A4 | BFC (50 μM), | 0 and 30 min, | HFC | Fluorimetry |
| Inhibition | NADP (1.3 mM), | 37° C. | | |
| (BFC substrate) | G6P (3.3 mM), | | | |
| | G6PDHase (0.4 U/mL) | | | |

Abbreviations:
BFC: 7-Benzyloxy-4-(trifluoromethyl)-coumarin; from Discovery Labware, catalog number 451730
CEC: 3-Cyano-7-ethoxycoumarin, from Molecular Probes, catalog number C-684
CHC: 3-Cyano-7-hydroxycoumarin
CYP: Cytochrome P450
G6P: D-Glucose-6-phosphate, from Sigma, catalog number G-7772
G6PDHase: Glucose-6-phosphate dehydrogenase, from Sigma, catalog number G-4134
HFC: 7-Hydroxy-4-trifluoromethylcoumarin
MFC: 7-Methoxy-4-trifluoromethylcoumarin, from Sigma, catalog number T-3165
NADP: β-Nicotinamide adenine dinucleotide phosphate, from Sigma, catalog number N-0505

Abbreviations: BFC: 7-Benzyloxy-4-(trifluoromethyl)-coumarin; from Discovery Labware, catalog number 451730

CEC: 3-Cyano-7-ethoxycoumarin, from Molecular Probes, catalog number C-684
CHC: 3-Cyano-7-hydroxycoumarin

### CYP: Cytochrome P450

G6P: D-Glucose-6-phosphate, from Sigma, catalog number G-7772
G6PDHase: Glucose-6-phosphate dehydrogenase, from Sigma, catalog number G-4134
HFC: 7-Hydroxy-4-trifluoromethylcoumarin
MFC: 7-Methoxy-4-trifluoromethylcoumarin, from Sigma, catalog number T-3165
NADP: β-Nicotinamide adenine dinucleotide phosphate, from Sigma, catalog number N-0505

Results ADME-Tox: In Vitro Metabolism. The mean values for the effects of 5-(5-cyano-1H-indol-1-yl)pentylboronic acid are summarized in Table 13. The data obtained with the reference compounds is shown Table 14.

TABLE 13

| | Test | % Inhibition |
|---|---|---|
| Assay | Concentration (M) | of Control Values |
| CYP1A2 Inhibition | 1.0E−05 | 40 |
| (CEC substrate) | | |
| CYP2C9 Inhibition | 1.0E−05 | 73 |
| (7-MFC substrate) | | |
| CYP2C 19 Inhibition | 1.0E−05 | 52 |
| (CEC substrate) | | |
| CYP2D6 Inhibition | 1.0E−05 | 42 |
| (7-MFC substrate) | | |
| CYP3A4 Inhibition | 1.0E−05 | 97 |
| (BFC substrate) | | |

TABLE 14

| Assay | IC₅₀ | |
|---|---|---|
| Reference Compound | (M) | n_{H} |
| CYP1A2 Inhibition (CEC substrate) | 5.5E−06 | 0.6 |
| furafylline | | |
| CYP2C9 Inhibition (7-MFC substrate) | 2.5E−07 | 1.0 |
| sulfaphenazole | | |
| CYP2C 19 Inhibition (CEC substrate) | 3.0E−06 | 0.7 |
| tranylcypromine | | |
| CYP2D6 Inhibition (7-MFC substrate) | 4.8E−08 | 0.9 |
| quinidine | | |
| CYP3A4 Inhibition (BFC substrate) | 7.3E−07 | 1.4 |
| ketoconazole | | |

ADME-Tox: For QT Prolongation the general procedure is shown in Table 15 and the experimental condition are shown in Table 16. In the event that a negative (<5% inhibition) compound was tested, the reference compound was perfused into the bath to ensure blockade of the HERG current, thereby eliminating false negative results. For positive (active) compounds, controls with 10 nM E-403 1 were performed in separate cells (same clone). E-4031: from Wako, catalog number 052-06523. For patch-clamp, the incubation conditions were applied until steady-state was achieved.

TABLE 15

| | | Reference | |
|---|---|---|---|
| Assay | Cells | Compound | Bibliography |
| K⁺ channel | HEK-293 cell line stably | E-4031 | Zhou et al. |
| (HERG) | expressing HERG | | (1998) |
| (patch-clamp) | | | |

TABLE 16

| | | | Method of |
|---|---|---|---|
| Assay | Incubation | Conditions (mM) | Detection |
| K⁺ channel | 10-20 min, | Pipette: 130 KC1, 10 NaCI, 1 MgC1₂, 10 | Whole-cell |
| (HERG) | 22-24° C. | EGTA, 5 MgATP, 10 HEPES (pH | patch-clamp |
| (patch- | | adjusted to 7.2 with 1 N KOH) | |
| clamp) | | Bath: 137 NaCl, 4 KC1, 1.8 CaC1₂, I MgC1₂, | |
| | | 10 D(+)-Glucose, 10 HEPES | |
| | | (pH adjusted to 7.4 with 1 N NaOH) | |

For HERG (patch-clamp) studies cultured cells (1-3 days) were used for recordings. The cells were cultured in DMEM/F 12+10% FBS. For recording, cells were plated on collagen-coated coverslips at low density (about 10⁴ cells/mL). The cells were held at −80 mV and depolarized to +20 mV for two seconds, followed by a one second pulse to −40 mV to reveal the tail current. This paradigm was delivered once every eight seconds (0.125 Hz) to monitor the current amplitude. After the current amplitude stabilized, the test compound was delivered to the extracellular medium by bath perfusion. During superfusion, the cell was repetitively stimulated with the protocol described above, and the current amplitude was continuously monitored. Data were acquired and analyzed by using pClamp (Axon Instruments) and Excel (Microsoft), and are reported as mean and individual values. The degree of inhibition (%) was obtained by measuring the tail current amplitude before and after drug perfusion (the difference current was normalized to control and multiplied by 100 to obtain the percent of inhibition).

Results. ADME-Tox: QT Prolongation Tables 17 contains the mean experimental values for the test compound. By adopting a general potency ranking system (Roche et al. ChemBioChem 2002, 3, 455-459) (Low, IC₅₀>10 μM; Moderate, 1 μM<IC₅₀<10 μM; and High, IC₅₀<1 μM), and based on the experimental findings, the test compound can be classified as a moderate-potency HERG-channel blocker.

TABLE 17

| | Inhibition of | |
|---|---|---|
| Test | Tail Current | |
| Concentration | (%) | Potency |
| (μM) | MEAN | Ranking |
| 1 | 31.1 | Moderate |

### Example 39

### The effect of the compound of Example 7 5-(2-(4-Methoxyphenyl)-1H-benzo[d]imidazol-1-yl)pentylboronic acid in various in vitro phosphodiesterase and ADME-Tox assays

The in vitro pharmacology of 5-(2-(4-Methoxyphenyl)-1H-benzo[d]imidazol-1-yl)pentylboronic acid was determined with several enzymes as described in Table 19 using the experimental conditions of Table 18.

TABLE 18

| | | Reference | |
|---|---|---|---|
| Assay | Origin | Compound | Bibliography |
| Phosphodiesterase 1 | bovine brain | 8-methoxy- | Nicholson et al. |
| | | IBMX | (1989) |
| Phosphodiesterase 2 (h) | differentiated | EHNA | Torphy et al. |
| | U-937 cells | | (1992) |
| Phosphodiesterase 3 (h) | human | milrinone | Weishaar et al. |
| | platelets | | (1986) |
| Phosphodiesterase 4 (h) | U-937 cells | rolipram | Torphy et al. |
| | | | (1992) |
| Phosphodiesterase 5 (h) | human | dipyridamole | Weishaar et al. |
| | platelets | | (1986) |
| Phosphodiesterase 6 | bovine retina | zaprinast | |

TABLE 19

| | Substrate/Stimulus/ | | Reaction | Method |
|---|---|---|---|---|
| Assay | Tracer | Incubation | Product | of Detection |
| Phosphodiesterase 1 | [³H]cAMP + | 30 min./ | [³H]5′ AMP | Scintillation |
| | cAMP | 30° C. | | counting |
| | (1 μM) | | | |
| Phosphodiesterase 2 (h) | [³H]cAMP + | 30 min./ | [³H]5′ AMP | Scintillation |
| | cAMP (1 μM) | 30° C. | | counting |
| Phosphodiesterase 3 (h) | [³H]cAMP + | 30 min./ | [³H]5′ AMP | Scintillation |
| | cAMP | 30° C. | | counting |
| | (0.1 μM) | | | |
| Phosphodiesterase 4 (h) | [³H]cAMP + | 30 min./ | [³H]5′ AMP | Scintillation |
| | cAMP | 30° C. | | counting |
| | (1 μM) | | | |
| Phosphodiesterase 5 (h) | [³H]cGMP + | 30 min./ | [³H]5′ GMP | Scintillation |
| | cGMP | 30° C. | | counting |
| | (1 μM) | | | |
| Phosphodiesterase 6 | [³H]cGMP + | 30 min./ | [³H]5′ GMP | Scintillation |
| | cGMP (2 μM) | 30° C. | | counting |

Results. The IC₅₀ values determined for 5-(2-(4-Methoxyphenyl)-1H-benzo[d]imidazol-1-yl)pentylboronic acid are indicated in Table 20. The corresponding inhibition curves were also determined (data not shown). The IC₅₀ value for each reference compound is indicated in Table 21. Each is within accepted limits of the historic average ±0.5 log units. Table 21 contains the mean experimental values for the QT prolongation study as performed as described above. By adopting a general potency ranking system (Roche et al. ChemBioChem 2002, 3, 455-459) (Low, IC₅₀>10 μM; Moderate, 1 μM<IC₅₀<10 μM; and High, IC₅₀<1 μM), and based on the experimental findings, the test compound can be classified as moderate/high-potency HERG-channel blocker.

TABLE 20
IC₅₀ Determination: Summary Results

| Assay | IC_{50 (M)} | n_{H} | Flags |
|---|---|---|---|
| Phosphodiesterase 1 | | | N.C. |
| Phosphodiesterase 2 (h) | 1.1E−07 | 0.8 | |
| Phosphodiesterase 3 (h) | | | N.C. |
| Phosphodiesterase 4 (h) | 4.2E−07 | 1.0 | |
| Phosphodiesterase 5 (h) | 9.6E−07 | 0.7 | |
| Phosphodiesterase 6 | 4.7E−06 | 0.9 | |

N.C. Not calculable.
IC50 value is not calculable because of less than 25% inhibition at the highest tested concentration.

TABLE 21

| | INHIBITION OF TAIL | |
|---|---|---|
| Test Concentration | CURRENT (%) | Potency |
| (μM) | MEAN | Ranking |
| 1 | 49.7 | Moderate/High |

### Example 40

### Biological Example

### Inhibition of TNF-α Production By Peripheral Blood Monocyte Cells (PMBC)

PMBC in RPMI 1640 Cell Culture Medium (containing 1% Penicillin and 1% Streptomycin) are aliquoted into 96-well plates at 5×10⁵ cells/well and pre-incubated with test compounds for 30 minutes at 37° C. After incubation, 1 ug/mL LPS is added to each well to stimulate TNF-α production and the plate is incubated for 24 hours at 37° C. After incubation, the supernatant is removed and the TNF-α secreted is quantified using EIA detection kits commercially available from R&D Systems (USA). The results from this assay are expressed as percent inhibition of control activity, with the control being stimulated wells with no test compound. Dexamethasone is used as a standard reference compound in the assay and is tested with each experiment. All test compounds are diluted from 10 mM stock solutions in 100% DMSO.

TABLE 22
TNF-α IC50 Values

| Example | | |
|---|---|---|
| Number(s) | Compound | IC₅₀ |
| 2-4 | 5-(2-(Thiazol-4-yl)-1H-benzo[d]imidazol-1-yl)pentylboronic acid | 560 nM |
| 7 | 5-(2-(4-Methoxyphenyl)-1H-benzo[d]imidazol-1-yl)pentylboronic | 200 nM |
| | acid | |
| 11 | 5-(2-(3-Fluoro-4-methoxyphenyl)-1H-benzo[d]imidazol-1- | 500 nM |
| | yl)pentylboronic acid | |
| 20 | 5-(5-cyano-1H-indol-1-yl)pentylboronic acid | 750 nM |
| 27 | 6-(5-cyano-1H-indol-1-yl)-N-hydroxyhexanamide | 900 nM |
| 30 | 5-(6-fluoro-2,3-dihydro-3-oxobenzo[b][1,4]oxazin-4- | 260 nM |
| | yl)pentylboronic acid | |
| 31 | 5-(2,3-dihydro-3-oxobenzo[b][1,4]thiazin-4-yl)pentylboronic | 700 nM |
| | acid | |
| 32 | 5-(7-chloro-2,3-dihydro-3-oxobenzo[b][1,4]thiazin-4- | 80 nM |
| | yl)pentylboronic acid | |
| 33 | 5-(2,3-dihydro-7-nitro-3-oxobenzo[b][1,4]oxazin-4- | 74 nM |
| | yl)pentylboronic acid | |
| 34 | 5-(2,3-dihydro-3-oxobenzo[b][1,4]oxazin-4-yl)pentylboronic acid | 360 nM |
| 35 | ethyl 2-(3,4-dihydro-3-oxo-4-(5-pentylboronic acid)-2H- | 810 nM |
| | benzo[b][1,4]thiazin-2-yl)acetate | |

### Example 41

### Effects of Several Compounds in Various In Vitro Cell Biology Assays

The procedures used in the various assays are shown in Table 23. In each experiment, the respective reference compound was tested concurrently with the test compounds in order to assess the assay suitability. It was tested at several concentrations (for IC₅₀ value determination).

TABLE 23

| | | Reference | |
|---|---|---|---|
| Assay | Origin | Compound | Reference |
| IFN-γ secretion (h) | PBMC | dexamethasone | Andre et al. (1996) |
| TNF-α secretion | PBMC | dexamethasone | Schindler et al. (1990) |
| (h) | | | |
| IL-1β secretion (h) | PBMC | cycloheximide | Schindler et al. (1990) |
| IL-2 secretion (h) | PBMC | dexamethasone | Konno et al. (1994) |
| IL-4 secretion (h) | PBMC | dexamethasone | Endo et al. (1993) |
| IL-6 secretion (h) | PBMC | dexamethasone | Schindler et al. (1990) |
| IL-10 secretion (h) | PBMC | dexamethasone | Rigano et al. (1995) |
| IL-8 secretion (h) | PBMC | dexamethasone | Schindler et al. (1990) |
| Cell viability (h) | PBMC | erythromycin | Mosmann (1983) |

The experimental condition used in the assays are shown in Table 24. The test compounds were assayed at 3×10⁻⁶ M.

TABLE 24

| | | | Reaction | Method of |
|---|---|---|---|---|
| Assay | Substrate/Stimulus/Tracer | Incubation | Product | Detection |
| IFN-γ secretion | PHA (2 μg/ml) | 24 h/37° C. | IFN-γ | EIA |
| (h) | | | | |
| TNF-α | LPS (1 μg/ml) | 24 h/37° C. | TNF-α | EIA |
| secretion (h) | | | | |
| IL-1β | LPS (1 μg/ml) | 24 h/37° C. | IL-1β | EIA |
| secretion (h) | | | | |
| IL-2 secretion | PHA (20 μg/ml) | 48 h/37° C. | IL-2 | EIA |
| (h) | | | | |
| IL-4 secretion | ConA (20 μg/ml) | 48 h./37° C. | IL-4 | EIA |
| (h) | | | | |
| IL-6 secretion | LPS (1 μg/ml) | 24 h./37° C. | IL-6 | EIA |
| (h) | | | | |
| IL-10 | PHA (3 μg/ml) | 48 h./37° C. | IL-10 | EIA |
| secretion (h) | | | | |
| IL-8 secretion | LPS (1 μg/ml) | 24 h./37° C. | IL-8 | EIA |
| (h) | | | | |
| Cell viability (h) | MTT (0.5 mg/ml) | 24 h./37° C. | formazan | Photometry |

Results. The mean values for the effects of the test compounds are summarized in tables 25. The results are expressed as a percent of control values and as a percent inhibition of control values obtained in the presence of the test compounds.

TABLE 25

| | | % Inhi- |
|---|---|---|
| | | bition |
| | | of |
| | Test | Control |
| Codes | Compound | Values |
| IFN-γ secretion (h) (PBMC) | | |
| 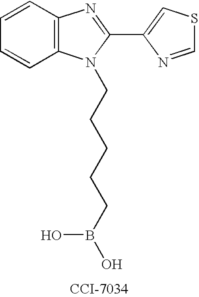 | | 21 |
| 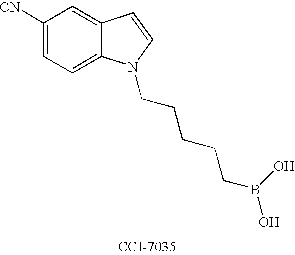 | | 16 |
| 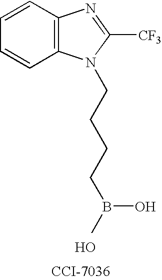 | | −6 |
| 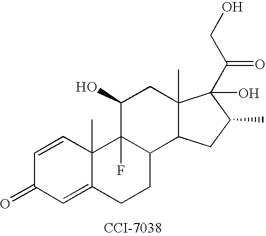 | | 68 |
| 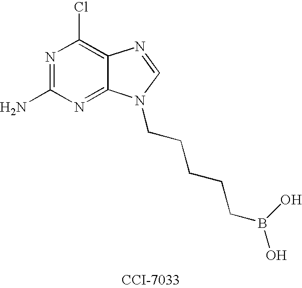 | | 56 |
| 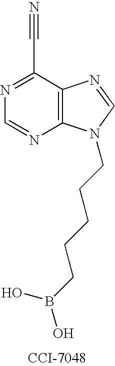 | | 14 |
| IL-1 □ secretion (h) (PBMC) | | |
| 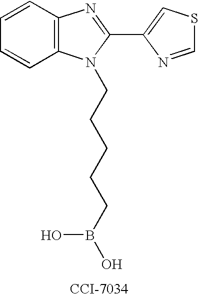 | | −67 |
| 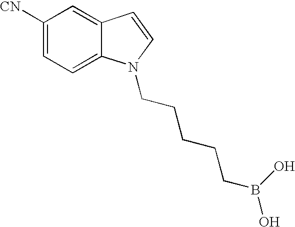 | | |
| 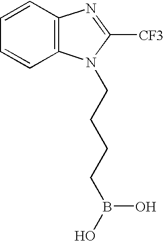 | | |
| 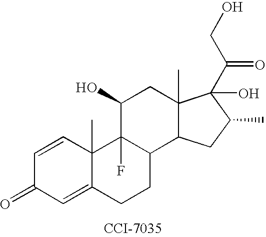 | | −12 |
| CCI-7036 | | 25 |
| CCI-7038 | | 68 |
| CCI-7033 | | −11 |
| 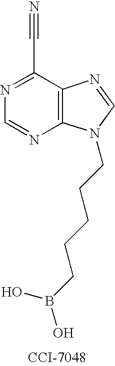 | | −66 |
| IL-2 secretion (h) (PBMC) | | |
| 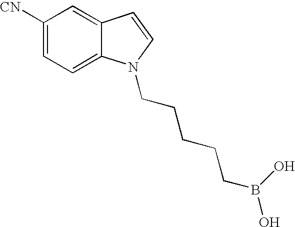 | | |
| 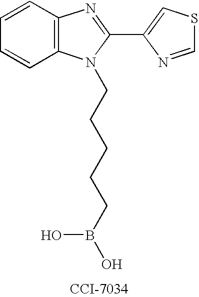 | | 56 |
| 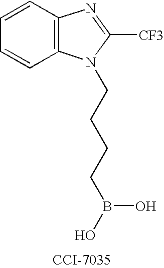 | | 72 |
| 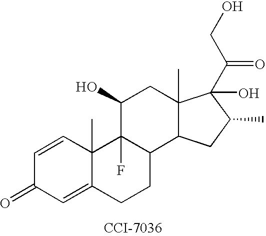 | | 18 |
| 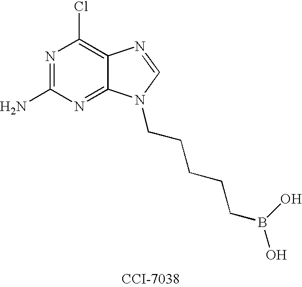 | | 78 |
| CCI-7033 | | 63 |
| CCI-7048 | | 23 |
| IL-4 secretion (h) (PBMC) | | |
| 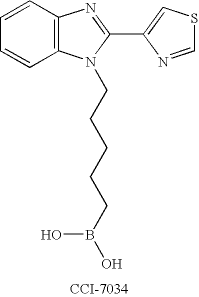 | | |
| 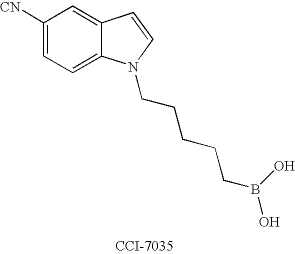 | | |
| 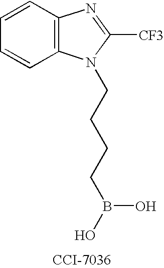 | | |
| 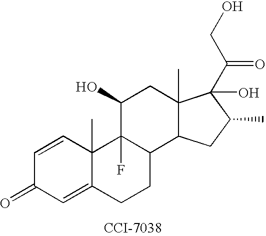 | | 118 |
| 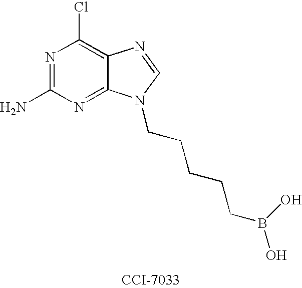 | | −30 |
| CCI-7048 | | -45 |
| IL-6 secretion (h) (PBMC) | | |
| CCI-7034 | | −89 |
| 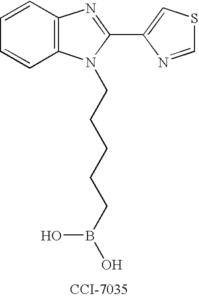 | | −61 |
| 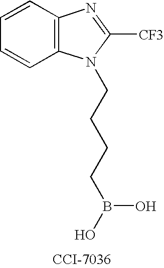 | | −26 |
| 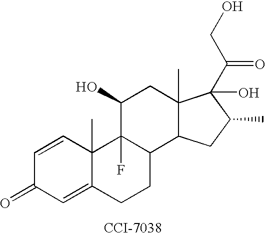 | | 57 |
| 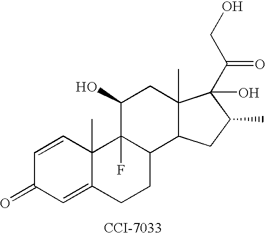 | | −70 |
| 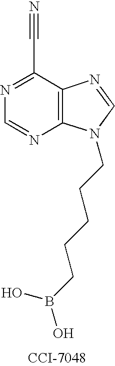 | | −30 |
| IL-10 secretion (h) (PBMC) | | |
| CCI-7034 | | 42 |
| 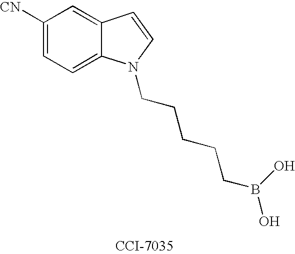 | | 36 |
| 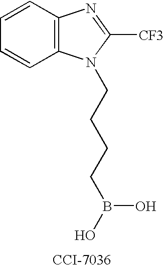 | | 17 |
| 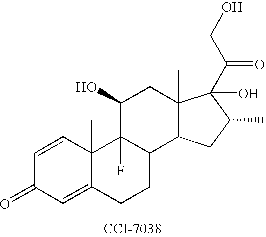 | | 62 |
| 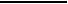 | | 50 |
| 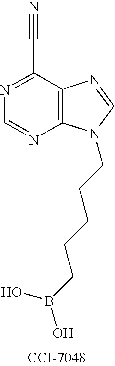 | | 13 |
| IL-8 secretion (h) (PBMC) | | |
| CCI-7034 | | −32 |
| 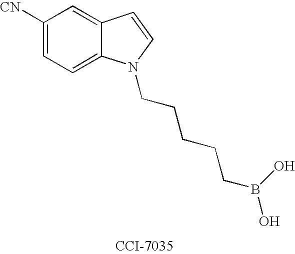 | | −63 |
| 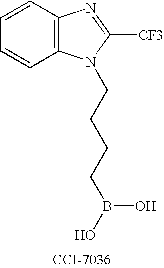 | | −29 |
| 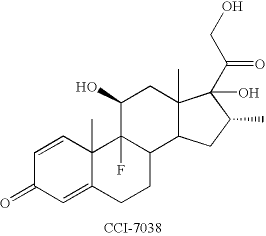 | | 67 |
| 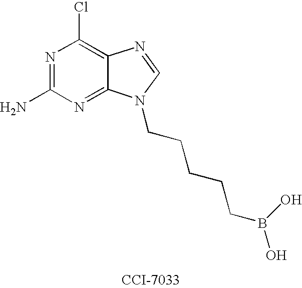 | | 8 |
| 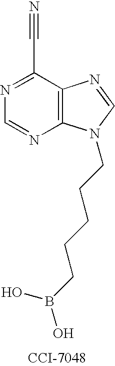 | | −33 |
| | | % |
| | | Cyto- |
| Assay | Compound | toxicity |

| | |
|---|---|
| Cell viability (h) (PBMC/24 h) | |
| CCI-7034 | −9 |
| 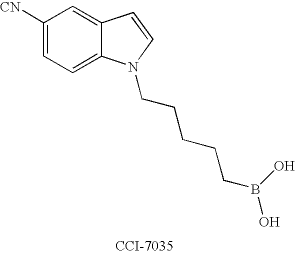 | 0 |
| 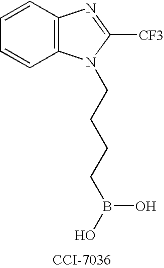 | −6 |
| 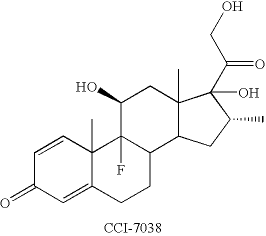 | 6 |
| 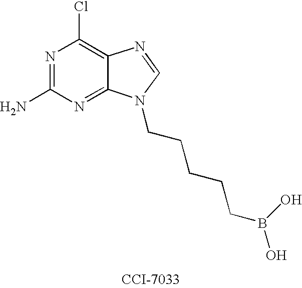 | 6 |
| 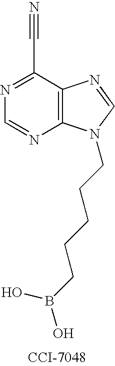 | 5 |

The IC₅₀ values (concentration causing a half-maximal inhibition of control values) and Hill coefficients (n_{H}) were determined by non-linear regression analysis of the inhibition curves using Hill equation curve fitting, a summary of the data is shown in Table 26. The IC₅₀ value for each reference compound is indicated was determined (data not shown) and was within accepted limits of the historic average ±0.5 log units.

TABLE 26

| Assay Compound | IC₅₀ | | Flags |
|---|---|---|---|
| TNF-α secretion (h) (PBMC) | | | |
| 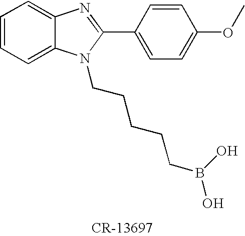 | 200 | (nM) | |
| 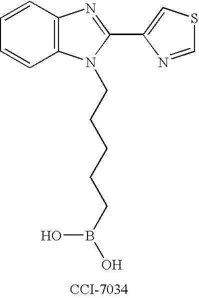 | | | >3.0E-06 (M) |
| 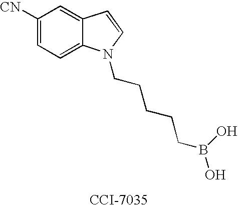 | 1.5E-06 | (M) | |
| 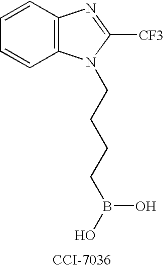 | | | N.C. |
| 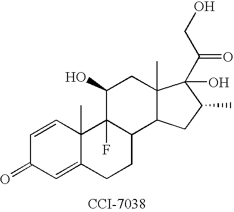 | 3.6E-08 | (M) | |
| CCI-7033 | 1.3E-06 | (M) | |

>Conc. Above the highest test concentration. IC50 value is above the highest tested concentration. Dose response curve has an inhibitory shape with less than 50% inhibition at the highest tested concentration
N.C. Not calculable. IC50 value is not calculable because of less than 25% inhibition at the highest tested concentration.

### Example 41

Effects of CCI-7155 and CCI-7156, and sulfasalazine, in a rat model of colitis provoked by challenge with trinitrobenzene sulphonic acid (TNBS).

The model of colitis that is provoked by intracolonic instillation of trinitrobenzene sulphonic acid (TNBS) as described by Morris and associates and Boughton-Smith and colleagues, and is now widely used and well-characterised (Boughton-Smith et al, 1988a,1998b; Morris et al, 1989; Reuter et al, 1996; Kiss et al, 1997; Fries et al, 1998; Galvez et al, 2000; Ballinger et al, 2000; Whittle et al, 2003). The inflammatory response provoked by TNBS is considered to reproduce many of the macroscopic, histological, and immunological hallmarks of clinical colitis. Thus, open ulceration may be produced, with transmural inflammation and thickening of the bowel wall. Histological features include distorted crypt architecture, crypt atrophy, granulomata, giant cells, basal lymphoid aggregates and the presence of an inflammatory infiltrate (Morris et al, 1989; Yamada et al, 1992; Hoffmann et al, 1997; Torres et al, 1999; Neurath et al, 2000, Villegas et al, 2003). Thus, the model has been used and validated for studying colonic inflammation and therefore to address aspects of the pathogenesis of IBD, as is the industry standard for evaluating potential novel therapeutic agents for this utility (Whittle et al. 2003).

In the present example, the effects of the following compounds were evaluated in the TNBS model.

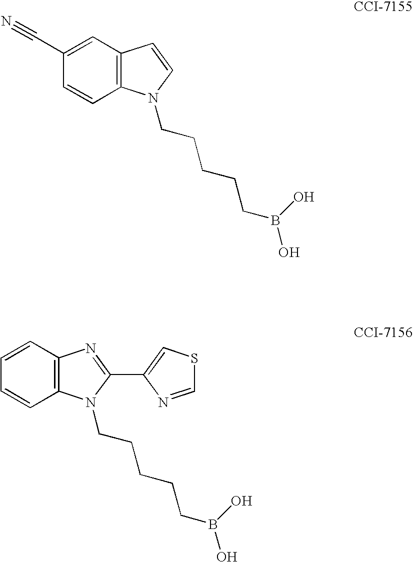

The effects of CCI-7155 and CCI-7156, were evaluated in the TNBS model at one and two oral dose levels, administered twice a day by gavage, treatment commencing 1 day prior to challenge. A low intracolonic concentration of TNBS (10 mg) was used, known to produce reproducible yet not unduly severe mucosal injury in the colon, determined 3 days after instillation. Colonic macroscopic injury has been assessed, as has colonic weight as a reflection of colonic oedema and wet/dry weight to determine colonic water content, along with determination of myeloperoxidase (MPO) activity (Bradley et al, 1982) as an index of white cell infiltration for the evaluation of tissue injury. In addition to the test compounds [CCI-7155 and CCI-7156], the actions of sulfasalazine, a well-established and currently used treatment has also been evaluated in this model.

### Methods and Protocol

TNBS Challenge—Male Wistar rats (230-280 g) were randomised into groups of 8-10 before commencement of the study. Food was withdrawn 18 h (overnight) before TNBS administration, but the rats were allowed free access to drinking water. On the morning of the day of challenge, Day 0, the rats were transiently anaesthetised with ether and TNBS (10 mg in 0.25 ml of 50% ethanol) was instilled approximately 6-8 cm into the colon using a soft plastic catheter inserted in the rat rectum. The rats were allowed to recover with free access to food and drinking water. At the end of the experiment, 72 h after TNBS administration (i.e. on the morning of Day 3, between 9.00 and 11.00), the distal colon was dissected, and the distal 8 cm photographed and stored appropriately for subsequent analyses.

The following primary parameters were measured in the main study: (a) macroscopic scoring of distal 8 cm of colon; (b) myeloperoxidase levels in segments of distal 8 cm of colon. In addition, the weight of the colonic segment was assessed as an indirect and non-specific marker of oedema, and this was supported by measurement of the wet/dry ratio as an index of water content. The body weight of the animals was also determined and expressed as % change from the day of challenge.

Treatments. All challenged groups were dosed orally twice daily from Day −1. The groups for study were:

(a) Vehicle control 0.5% carboxy methyl cellulose (CMC) p.o., twice daily from Day −1

(b) sulfasalazine 25 mg/kg, p.o., twice daily from Day −1 (50 mg/kg/day total)

(c) CCI-7155 25 mg/kg, p.o., twice daily from Day −1 (50 mg/kg/day total)

(d) CCI-7155 50 mg/kg, p.o., twice daily from Day −1 (100 mg/kg/day total)

(e) CCI-7156 50 mg/kg, p.o., twice daily from Day −1 (100 mg/kg/day total)

A further group of non-challenged and non-treated animals was used for baseline measurement of colonic MPO.

The compounds were thus administered orally, twice daily and given on Day −b 1 before TNBS administration, on Day 0, the day of TNBS administration and on Day 1 and 2. Tissues were removed 72 h after TNBS administration (on Day3). Dosing was performed once in the morning (Between 9:00 and 11:00) and once in the late afternoon (between 18:00 and 20:00).

Preparation of Compound. CCI-7155 and CCI-7156 were suspended in 0.5% w/v carboxy methyl cellulose in sterile water, to produce a smooth suspension as instructed, and administered in a volume of 2 ml/kg (˜0.5 ml per rat per dose).

Preparation of Sulfasalazine. the Doses of Sulfasalazine Used in the Present study were derived from previously published work with this agent in the TNBS model (Boughton-Smith et al, 1988; Sykes et al, 1999; Galvez et al, 2000; Bobin-Dubigeon et al, 2001). Sulfasalazine was suspended in carboxy methyl cellulose (0.5% w/v in sterile water; Sigma Chemical Co, compound reference M-0262) and administered p.o. in a volume of 0.5 ml. In previous studies in these laboratories, this concentration of carboxy methyl cellulose (CMC) had no significant effect on the extent of colitis as determined by macroscopic injury and changes in inflammatory markers following TNBS challenge.

Animal Husbandry: Male Wistar rats (270±30 g body weight) were used throughout.

Rats were maintained in air-conditioned with 20 air changes per hour and constantly monitored environment with temperature 21+2° C. The rooms were illuminated by fluorescent light on a 12 hour light/dark cycle, fed pelleted rat No. 1 maintenance diet RM1(E) and water ad libitum. Rats were housed in groups of 3-5 in polypropylene cages with animal bedding of graded cellulose wood fibres.

Macrocopic Injury. The distal 8 cm portion of the colon (measured from the rectum) was removed, opened longitudinally and gently rinsed with ice-cold phosphate buffer (PBS; pH 7.4), blotted, weighed (Scaltec, Germany) and photographed (Samsung, Digimax 340, digital camera). The tissue was then cut into longitudinal strips, each strip being thus 8 cm long and included the whole of the zone of injury. Each tissue was weighed and stored at −30° C. for the subsequent determination of myeloperoxidase activity, while a segment was also dried at 120° C. for 24 h for the determination of wet weight/dry weight ratio.

The extent of macroscopically apparent damage, involving regions of haemorrhagic necrosis, was determined in a randomised manner from the colour images via computerised planimetry (Scion Image B4.02 version; Scion Corp.). Data on the macroscopic measurements are shown in the Appendix I. Examples of the macroscopic appearance of the colon following challenge are shown in the Appendix II. The area of macroscopically visible mucosal damage was calculated and expressed as the percentage of the total colonic segment area under study.

Myeloperoxidase Activity. The myeloperoxidase activity was determined using the method described by Bradley (Bradley et al, 1982) with minor modifications. The 8 cm longitudinal strips of the colon were weighed, homogenised (Ultra turrax, T25, 2×30 sec; 250 mg colon/1 ml buffer) in ice-cold phosphate buffer (50 mM, pH 6.0), freeze thawed three times and centrifuged (15,000×g 15 min. at 4° C.). A 12 μl aliquot of the supernatant was mixed with 280 μl phosphate buffer (50 mM, pH 6) containing 0.167 mg/ml of O-adenosine dihydrochloride and the reaction started with 10 μl 0.03% hydrogen peroxide and assayed spectrophotometrically (Benchmark Microplate reader, Bio-Rad Lab.; λ=490 nm) after 90 sec. shaking. The standards used for preparation of the standard curve were 0, 0.05, 0.1, 0.2, 0.3, 0.4 and 0.5 U peroxidase/ml phosphate buffer. Myeloperoxidase activity (MPO) was expressed as mU/mg protein or wet weight of tissue.

Protein determination. The method used is that described by Bradford (Bradford, 1976). Thus, 20 μl of the diluted samples (25× or 50× with distilled water) was mixed with 980 μl distilled water and 200 μl Bradford reagent added to each sample. After mixing and a 10 min incubation, the samples were assayed spectrophotometrically (Benchmark Microplate reader, Bio-Rad Lab; λ=595 nm). The standard curve was 0, 2, 4, 6, 8 and 101 bovine serum albumin/ml distilled water. Protein was expressed as mg protein/ml.

Reagents and Materials. Trinitrobenzene sulphonic acid was obtained from Fluka (Chemie AG, Buchs, Switzerland). The Bradford protein assay was from BIO-RAD. All other assay reagents were from Sigma Chemical Company.

Statistical Evaluation. Results shown in the figures are expressed as mean±S.E.M. from n rats per experimental group. For statistical comparisons, the two-tailed Student's t-test and the analysis of variance with the Bonferoni test were used, where appropriate. P<0.05 was taken as significant.

### Results

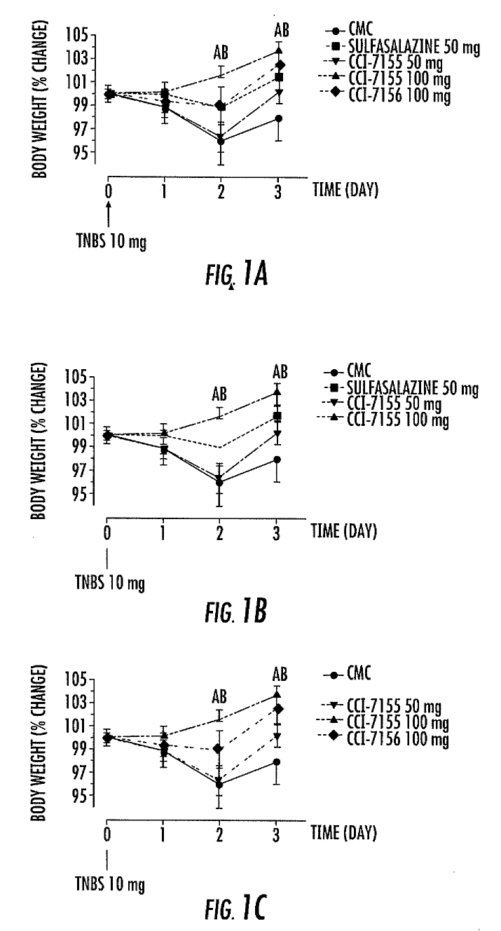

In FIGS. 1-5 the labels have the following meanings:
TNBS=2,4,6-Trinitrobenzenesulfonic acid solution (10 mg);
CMC=carboxy methyl cellulose vehicle;
CMC group=TNBS+0.5% CMC (0.5 ml/rat p.o.);
Sulfasalazine=TNBS+Sulfasalazine treated group (50 mg/kg/day p.o.)
CCI-7155 50=TNBS+CCI-7155 treated group (50 mg/kg/day p.o.)
CCI-7155 100=TNBS+CCI-7155 treated group (100 mg/kg/day p.o.)
CCI-7156 100=TNBS+CCI-7156 treated group (100 mg/kg/day p.o.)
Control=non-treated, non-challenged absolute control.

Body Weight. FIGS. 1A-C show the effects of CCI-7155 (50 and 100 mg/kg/day p.o.), CCI-7156 (100 mg/kg/day p.o.) and sulfasalazine (50 mg/kg/day p.o.) on body weight, expressed a % change in body weight at Day 0. Compounds were given in divided doses in a twice a day dosing schedule. Results are expressed as mean S.E.M.; n=9-10; significance is shown as aP<0.05 compared with 0.5% CMC group bP<0.05 compared with CCI-7155 50 mg group.

Following challenge with TNBS, there was a fall in body weight observed in the vehicle-challenge control group over the 3 day period, with the fall in body weight reaching its peak after 2 days (FIGS. 1A-C). In contrast, there was no fall in body weight in the absolute control group that received no treatment nor was challenged with TNBS (data not shown). Treatment with CCI-7155 (50 and 100 mg/kg/day, administered orally in divided doses of 25 and 50 mg/kg b.i.d respectively) caused a dose-dependent attenuation of this fall in body weight, as shown in FIGS. 1A-C. The effects of the higher dose of CCI-7155 were significantly different from the TNBS control group at both Day 2 and Day 3 post-challenge (P<0.05). The effects of CCI-7156 (100 mg/kg/day administered orally in divided doses of 50 mg/kg b.i.d) reached marginal significance (P<0.056) at Day 3 post-challenge (data not shown). Treatment with sulfasalazine (50 mg/kg/day administered orally in divided doses of 25 mg/kg b.i.d), while appearing to attenuate the body weight loss (FIGS. 1A-B), did not reach statistical significance for this action at any of the time points (data not shown).

Macroscopic Colonic Injury. In this study following intracolonic instillation of TNBS (10 mg), the area of colonic injury, determined 72 h after challenge in the control group of rats that had only received the 0.5% CMC vehicle p.o. involved 26±3% (n=9) of the total colonic area of the segment studied, determined by computerized planimetric measurement. There was no detectable macroscopic injury in the colons from the non-challenged group of rats (data not shown). The macroscopic appearance of the colonic mucosa following challenge and with the various treatments was assessed (data not shown). FIG. 2 shows the effects of CCI-7155 (50 and 100 mg/kg/day p.o.), CCI-7156 (100 mg/kg/day p.o.) and sulfasalazine (50 mg/kg/day p.o.) on macroscopic injury in the colon. Results are expressed as mean±S.E.M.; n=9-10; ^{a}p<0.05 compared with 0.5% CMC group ^{b}P<0.05 compared with CCI-7155 50 mg group ^{c}P<0.05 compared with Sulfasalazine 50 mg group.

Treatment with CCI-7155 (50 and 100 mg/kg/day administered orally in divided doses) caused a dose-dependent reduction in the area of colonic injury (FIG. 2). This reduction in TNBS-induced colonic damage was statistically significant for both doses (P<0.001 and P<0.0001 respectively) as shown in FIG. 2.

Treatment with CCI-7156 (100 mg/kg/day administered orally in divided doses) caused a reduction in the area of colonic injury (FIG. 2). This reduction in TNBS-induced colonic damage was statistically significant (P<0.001) as shown in FIG. 2.

Treatment with sulfasalazine (50 mg/kg/day administered orally in divided doses) also significantly (P<0.001) reduced the extent of macroscopic injury, as shown FIG. 2.

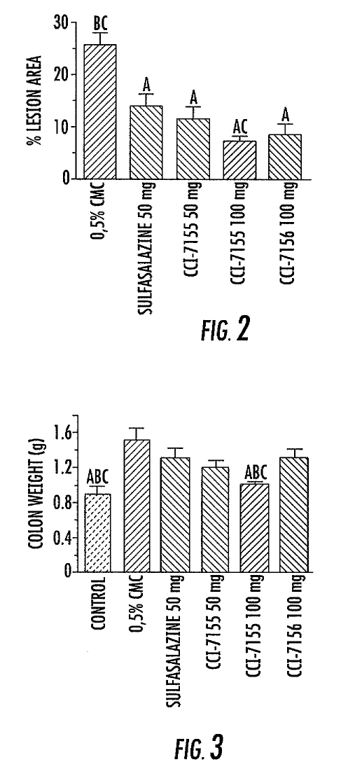

Colon Weight. As an indirect index of inflammatory oedema in the colonic tissue, the weight of the colonic segments was determined at the end of the study. FIG. 3 shows the effects of CCI-7155 (50 and 100 mg/kg/day p.o.), CCI-7156 (100 mg/kg/day p.o.) and sulfasalazine (50 mg/kg/day p.o.) on colon weight. Compounds were given in divided doses in a twice a day dosing schedule. Results are expressed as mean±S.E.M.; n=9-10; ^{a}P<0.05 compared with 0.5% CMC group ^{b}P<0.05 compared with CCI-7155 50 mg group ^{c}P<0.05 compared with Sulfasalazine 50 mg group.

As shown in FIG. 3, the colonic weight in the groups challenged with TNBS was significantly higher than that of non-challenged colon (absolute control) for a comparable tissue section. Treatment with CCI-7155 caused a dose-dependent reduction in the colon weight (FIG. 3). With the higher dose of CCI-7155, the reduction in the colonic weight of the standard segment was statistically significant, whereas that achieved by the lower dose was not (FIG. 3). Treatment with CCI-7156 did not cause a significant reduction in the colon weight (FIG. 3). A significant reduction in colon weight was also not observed in the sulfasalazine group (FIG. 3), despite the reduction in damage seen in those tissues.

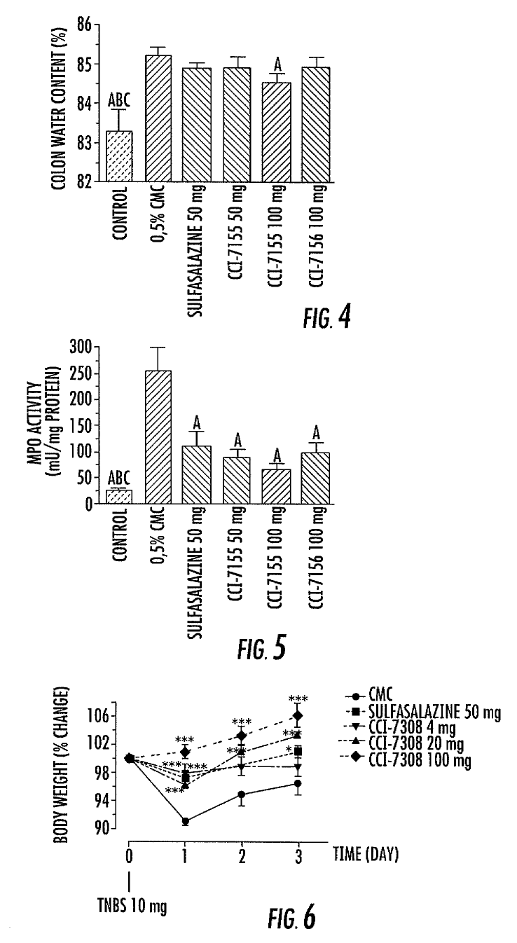

Colon Wet/Dry Weight. As an index of water content in the colonic tissue, the weight of the colonic segments was determined at the end of the study both wet and after oven-drying. FIG. 4 shows the effects of CCI-7155 (50 and 100 mg/kg/day p.o.), CCI-7156 (100 mg/kg/day p.o.) and sulfasalazine (50 mg/kg/day p.o.) on water content in the colon. Compounds were given in divided doses in a twice a day dosing schedule. Results are expressed as mean±S.E.M.; n=9-10; ^{a}P<0.05 compared with 0.5% CMC group ^{b}P<0.05 compared with CCI-7155 50 mg group ^{c}P<0.05 compared with Sulfasalazine 50 mg group.

As shown in FIG. 4, the colonic water content in the groups challenged with TNBS was significantly higher than that of non-challenged colon (absolute control) for a comparable tissue section. As with the colon weight, treatment with CCI-7155 caused a dose-dependent reduction in the colonic water content (FIG. 4). With the higher dose of CCl-7155, the reduction in the colonic water content was significant, whereas that achieved by the lower dose was not (FIG. 3). Treatment with CCI-7156 did not cause a significant reduction in the colonic water content (FIG. 4). Likewise, a significant reduction in colon weight was also not observed in the sulfasalazine group (FIG. 4), again despite the reduction in damage seen in those tissues.

Colonic MPO Levels. FIG. 5. shows the effects of CCI-7155 (50 and 100 mg/kg/day given p.o. in divided doses, b.i.d.), CCI-7156 (100 mg/kg/day given p.o. in divided doses, b.i.d.) and sulfasalazine (50 mg/kg/day given p.o. in divided doses, b.i.d) on MPO levels in the colon, expressed as mU/mg protein. Compounds were given in divided doses in a twice a day dosing schedule. Results are expressed as mean±S.E.M.; n=9-10; aP<0.05 compared with 0.5% CMC group bP<0.05 compared with CCI-7155 50 mg, cP<0.05 compared with Sulfasalazine 50 mg group

The level of MPO activity determined in the colonic tissue from rats in the unchallenged control group was significantly increased in the TNBS-challenged group (from 28±4 to 254±48 mU/mg protein; P<0.001), as shown in FIG. 5. Treatment with CCI-7155 caused a dose-dependent fall in the elevated MPO levels, with a significant (P<0.01) reduction in colonic MPO levels at both doses, as shown in FIG. 5. Likewise, treatment with CCI-7156 caused significant fall in the elevated MPO levels (FIG. 5). Treatment with sulfasalazine significantly reduced the elevated colonic levels of MPO as can be seen in FIG. 5. The extent of this reduction in MPO levels was in the same range as that brought about by the two experimental compounds (data not shown). The data for MPO has also been expressed as mU/g wet tissue (data not shown) and the relative changes between the groups were identical.

As described above, the intra-colonic instillation of TNBS (10 mg), caused a subchronic colitis in the rat. This macroscopic injury in the colon, determined 72 h after challenge, consists of areas of haemorrhagic necrosis, with evidence of tissue inflammation and hyperaemia. In the present study, the degree of macroscopic injury involved a mean of 25% of the measured colonic mucosa. Such a moderate degree of injury is useful in the first stage analysis of novel therapeutic compounds for this utility, as it allows sensitive detection of any preventative activity on the lesion development.

Oral administration of novel compound CCI-7155 caused a significant dose-dependent fall in the extent of macroscopically assessed TNBS-induced colonic damage, as did the single dose level of CCI-7156 evaluated.

The macroscopic injury provoked by TNBS was accompanied by a substantial increase in the levels of MPO in the colon, which is an index of leukocyte infiltration into the inflamed tissue (Morris et al, 1989; Reuter et al, 1996; Kiss et al, 1997). As with the macroscopic injury, both CCI-7155 and CCI-7156 caused significant reduction in colonic MPO activity, and the relative activity of these compounds on this parameter appeared comparable to that on macroscopic injury.

The elevated weight of the colonic segments following TNBS challenge, as an indirect index of oedema, was also dose-dependently reduced by the daily treatment with CCl-7155, a significant effect being observed at the higher dose. Likewise, the wet/dry ratio of the colon segment as an index of water content, was also significantly reduced by the higher dose of CCI-7155. This may reflect actions of this agent on the generalised inflammatory response in the tissue, with white-cell infiltration and oedema being attenuated. Despite the macroscopic injury being attenuated, none of the other treatment groups showed a reduction in these latter indirect parameters.

The fall in body weight that followed the challenge with TNBS was attenuated by CCI-7155 in a dose-dependent manner, with the higher dose of CCI-7155 preventing the fall in body weight at Day 2 and 3 post challenge. Although the effect of CCI-7156 on body weight change at Day 3 was near-significant, the data in the other groups was suggestive of an effect at some time-points, but this did not reach statistical significance.

Although sulfasalazine was introduced in clinical practice in the 1940's, the precise mechanism of its therapeutic action is a continuing discussion point but is, as yet, still unclear. It is known that the compound acts as a pro-drug, arriving essentially unchanged to the colon where it is cleaved by indigenous bacteria into its two constituent products, sulfapyridine and 5-aminosalicylic acid (5-ASA, mesalamine) by action on its azo linkage. It is considered that 5-ASA is the active moiety, being released in high concentrations locally, and a number of delivery formulations of 5-ASA are in current clinical use (Schroeder, 2003). Despite this widespread clinical use, experimental studies with sulfasalazine have produced inconsistent findings of efficacy in a range of IBD models, including TNBS-induced colitis. Thus, this compound has been found to have variable effects on several of the indices of TNBS-colitis, depending on the dose and schedule utilized (Boughton-Smith et al, 1988a; Sykes et al, 1999; Galvez et al, 2000; Bobin-Dubigeon et al, 2001). Studies on the putative active species, 5-ASA are even less clear as to the activity and reproducibility of effects in colitis models (Galvez et al, 2000; Tozaki et al, 2002).

Regarding the dose of sulfasalazine used in the current study, the clinical dose for the 500 mg tablets of the marketed form, Salazopyrin™, is 2-4 tablets×4 times a day for the treatment of active disease in IBD. Thus, this is a dose range of 4-8 g/day; based on an average body weight of 75 kg, the lower dose is thus 53 mg/kg/day. Indeed, the paediatric doses are given as 40-60 mg/kg/day for acute flare-up. Although pharmacokinetic differences between rat and humans have to be taken into account, the dose level used in the rats is thus close that that used in therapeutics.

In the present work, sulfasalzine at the dose of 50 mg/kg/day significantly reduced the degree of colonic injury and reduced the elevated MPO levels in the colonic tissue. As can be seen from the data, the activity of CCI-7155 and CCI-7156 on both parameters was comparable to that of sulfasalazine.

### Example 42

### The Effects of the CCI-7308 or Sulfasalazine in a Rat Model of Colitis Provoked by Trinitrobenzene Sulphonic Acid (TNBS)

In the present example, the effects of the following compound CCI-7308 was evaluated in the TNBS model.

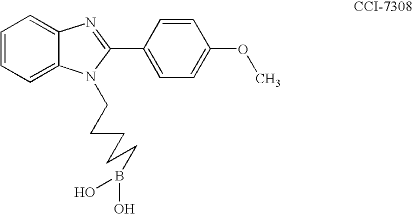

In the study a low intracolonic concentration of TNBS (10 mg) was used, known to produce reproducible yet not unduly severe mucosal injury in the colon, determined 3 days after instillation. In the study, colonic macroscopic injury has been assessed, as has colonic weight as a reflection of colonic oedema, along with determination of MPO activity (Bradley et al, 1982) as an index of white cell infiltration for the evaluation of tissue injury.

The pathogenesis of the inflammatory bowel diseases, including ulcerative colitis and Crohn's disease, is still not fully understood but it is likely that pro-inflammatory cytokine release and derangement of the immune response play a role in the inflammatory processes (Kappeler & Mueller, 2000; Papadakis et al, 2000). The colonic levels of the cytokine, tumour necrosis factor-α (TNF-α) have been shown to be increased in TNBS-induced colitis (Ameho et al, 1997; Ribbons et al, 1997; Sykes et al, 1999; Sun et al, 2001; Ten Hove et al, 2001; Villegas et al, 2003). Pharmacological studies using a number of putative inhibitors of the synthesis of TNF-α have suggested efficacy in reducing damage in TNBS-induced colitis in the rat (Bobin-Dubigeon et al, 2001). In the current study, the colonic levels of TNF-α after TNBS challenge have therefore also been determined.

### Methods and Protocol

The methods and protocol used were substantially similar to those in Example 39, with some differences described below.

TNBS Challenge—Male Wistar rats (270-330 g) were randomised into groups of 10-11 before commencement of the study.

The following primary parameters were measured in the study:

(a) macroscopic scoring of distal 8 cm of colon

(b) myeloperoxidase levels in segments of distal 8 cm of colon

(c) TNF-α levels in segments of distal 8 cm of colon.

In addition, the weight of the colonic segment was assessed as an indirect and non-specific marker of oedema. The body weight of the animals was also determined and expressed as % change from the day of challenge.

Treatments. All challenged groups were dosed orally twice daily from Day −1. The groups for study were:

(a) Vehicle control 0.5% carboxy methyl cellulose (CMC) p.o., twice daily from Day −1

(b) sulfasalazine 25 mg/kg, p.o., twice daily from Day −1 (50 mg/kg/day total)

(c) CCI-7308 2 mg/kg, p.o., twice daily from Day −1 (4 mg/kg/day total)

(d) CCI-7308 10 mg/kg, p.o., twice daily from Day −1 (20 mg/kg/day total)

(e) CCI-7308 50 mg/kg, p.o., twice daily from Day −1 (100 mg/kg/day total).

Colon homogenates for cytokine measurements. The colonic tissue samples were thawed, weighed and homogenized (Ultra-turrax, T25, 2×30 sec on ice) in 4 volumes (250 mg colon/ml buffer) of a modified a Greenburg buffer (300 mmol/L NaCl, 15 mmol/L Tris, 2 mmol/L MgCl, 2 mmol/L Triton X-100, 20 ng/ml pepstatin A, 20 ng/ml leupeptin, 20 ng/ml aprotonine; pH: 7.4). Tissue homogenates were lysed for 30 min. on ice, and then centrifuged (10 min., 14, 000×g). The aliquots of the supernatant were stored at −20° C. until use (Ten Hove et al., 2001).

Tumour Necrosis Factor-a Activity. The TNF-α levels were determined with quantitative TNF-α solid-phase Enzyme Linked ImmunoSorbent Assay (ELISA), which is based on the sandwich principle (HyCult biotechnology b. V;. Cat number: HK102). The TNF-α standards used were 0, 8.2, 20.5, 51.2, 128, 320, 800 and 2000 pg/ml. At the end of the ELISA assay, the samples were measured spectrophotometrically (Benchmark Microplate reader, Bio-Rad Lab; λ=450 nm). The samples were diluted 2 or 4 times with the sample buffer included in the kit. The TNF-α values were expressed as pg/mg protein. This commercially available kit (Hycult Biotechnology b.v. Uden, The Netherlands. Catalogue number: HK10k) used had a range of the standard curve of 0-2000 pg/ml with minimum detection level of 10 pg/ml of TNF-α.

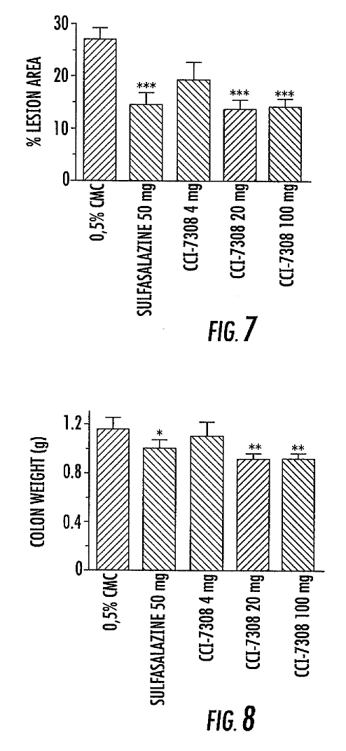

RESULTS. In FIGS. 6-9 the labels have the following meanings:

TNBS=2,4,6-Trinitrobenzenesulfonic acid solution (10 mg)

CMC=carboxy methyl cellulose vehicle

CMC=TNBS+0.5% CMC (0.5 ml/rat p.o.)

Sulfasalazine=TNBS+Sulfasalazine treated group (50 mg/kg/day p.o.)

CCI-7308 4=TNBS+CCI-7308 treated group (4.0 mg/kg/day p.o.)

CCI-7308 20=TNBS+CCI-7308 treated group (20 mg/kg/day p.o.)

CCI-7308 100=TNBS+CCI-7308 treated group (100 mg/kg/day p.o.)

Body Weight. Effects of CCI-7308 (4, 20 and 100 mg/kg/day p.o.) or sulfasalazine (50 mg/kg/day p.o.) on body weight, expressed a % change in body weight at Day 0. Compounds were given in divided doses in a twice a day dosing schedule. Results are expressed as mean±S.E.M.; n=9-11; *P<0.05, **P<0.01, ***P<0.001 compared with CMC group.

Following challenge with TNBS, there was a fall in body weight observed in the CMC vehicle-challenge control group over the 3 day period, with the fall in body weight reaching its peak after the first day post-challenge (FIG. 6). Treatment with CCI-7308 (4, and 100 mg/kg/day, administered orally in divided doses of 2, 10 and 50 mg/kg b.i.d respectively) caused a dose-dependent attenuation of this fall in body weight, as shown in FIG. 6. The effect of the lower dose of CCI-7308 was significantly different from the challenged CMC control group on Day 1, while the intermediate dose was significantly different from the CMC group on Days 1, 2 and 3 (P<0.001) as shown in FIG. 6.

The effects of CCI-7156 (100 mg/kg/day administered orally in divided doses of 50 mg/kg b.i.d) also were significant (P<0.001) on Days 1, 2 and 3 post-challenge (data not shown). Treatment with sulfasalazine (50 mg/kg/day administered orally in divided doses of 25 mg/kg b.i.d), attenuated the body weight loss following TNBS challenge (FIG. 6), which reached statistical significance at Day 1, 2 and 3 (data not shown).

Macroscopic Colonic Injury. FIG. 7 shows the effects of CCI-7038 (4, 20 and 100 mg/kg/day p.o) or sulfasalazine (50 mg/kg/day p.o.) on macroscopic injury in the colon. Compounds were given in divided doses in a twice a day dosing schedule. Results are expressed as mean±S.E.M.; n=9-11; ***P<0.001 compared with CMC group

In this study following intracolonic instillation of TNBS (10 mg), the area of colonic injury, determined 72 h after challenge in the control group of rats that had only received the 0.5% CMC vehicle p.o. involved 27±3% (n=11) of the total colonic area of the segment studied, determined by computerized planimetric measurement. There was no detectable macroscopic injury in the colons from a non-challenged group of rats (data not shown). The macroscopic appearance of the colonic mucosa following challenge and with the various treatments was determined.

Treatment with CCI-7308 (4, 20 and 100 mg/kg/day administered orally in divided doses) caused a dose-dependent reduction in the area of colonic injury (FIG. 7). This reduction in TNBS-induced colonic damage was statistically significant for both the and 100 mg/kg/day doses (P<0.001 for both), whereas that for the lower dose did not reach significance, as shown in FIG. 7. The data suggests that the maximal effect on this parameter was achieved with the intermediate dose of 20 mg/kg/day (FIG. 7), there being no significant effect between the actions of 20 and 100 mg/kg/day (FIG. 7). Treatment with sulfasalazine (50 mg/kg/day administered orally in divided doses) also significantly (P<0.001) reduced the extent of macroscopic injury, as shown in FIG. 7. The degree of inhibition with sulfasalazine was comparable to that achieved with the intermediate dose of CCI-7308 of 20 mg/kg/day (FIG. 7).

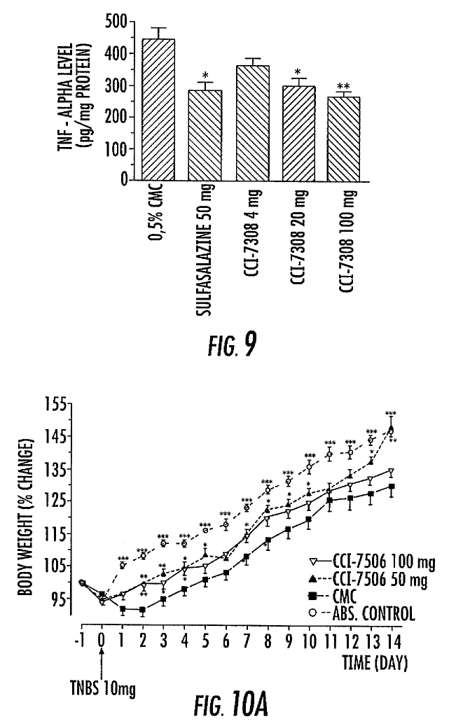

Colon Weight. FIG. 8 shows the effects of CCI-7038 (4, 20 and 100 mg/kg/day p.o) or sulfasalazine (50 mg/kg/day p.o.) on colon weight. Compounds were given in divided doses in a twice a day dosing schedule. Results are expressed as mean±S.E.M.; n=9-11;

*P<0.05, **P<0.01 compared with CMC group.

As an indirect index of inflammatory oedema in the colonic tissue, the weight of the standard colonic segments was determined at the end of the study. Treatment with CCl-7308 caused a dose-dependent reduction in the colon weight (FIG. 8). With the intermediate and higher dose of CCI-7308, the reduction in the colonic weight of the standard segment was statistically significant, whereas that achieved by the lower dose was not (FIG. 8). A significant (P<0.05) reduction in colon weight was also observed in the sulfasalazine group (FIG. 8).

Colonic MPO Levels. FIG. 8 shows the effects of CCI-7038 (4, 20 and 100 mg/kg/day p.o) or sulfasalazine (50 mg/kg/day p.o.) on MPO activity in the colon, expressed as mU/mg protein. Compounds were given in divided doses in a twice a day dosing schedule. Results are expressed as mean±S.E.M.; n=9-11; ***P<0.001 compared with CMC group. The level of MPO activity determined in the colonic tissue from the TNBS-challenged group was 273±25 mU/mg protein, as shown in FIG. 8). In a separate control study with colonic tissue from non-treated, non-challenged rats, the basal MPO activity was 44±12 mU/mg protein (n=11), significantly lower than that determined following TNBS challenge.

Treatment with CCI-7308 (4, 20 and 100 mg/kg/day) caused a dose-dependent fall in the elevated MPO activity, with a significant (P<0.001) reduction in colonic MPO levels at all three dose levels, as shown in FIG. 8. Treatment with sulfasalazine significantly reduced the elevated colonic levels of MPO as can be seen in FIG. 8. The extent of this reduction in MPO levels by sulfasalazine was, however, significantly less than that brought about by the higher dose of CCI-7308 (data not shown).

The data for MPO has also been expressed as mU/g wet tissue (data not shown), the relative changes between the groups were identical.

Colonic TNF-α Levels. FIG. 9 shows the effects of CCI-7038 (4, 20 and 100 mg/kg/day p.o) or sulfasalazine (50 mg/kg/day p.o.) on TNF-α levels in the colon, expressed as pg/mg protein. Compounds were given in divided doses in a twice a day dosing schedule. Results are expressed as mean±S.E.M.; n=9-11; *P<0.05, **P<0.01, compared with CMC group

The level of TNF-α in the colonic tissue from TNBS-challenged rats, determined after 3 days was 445±49 pg/mg protein (FIG. 9). In a separate control study with colonic tissue from non-treated, non-challenged rats, the basal TNF-α level was 16±4 pg/mg protein (n=11), substantially lower than that determined in colonic tissue following TNBS challenge.

Treatment with CCI-7308 dose-dependently reduced the level of TNF-α in the colonic tissues, with the effects of the intermediate and higher dose of achieving significance (FIG. 9), while those of the lower dose did not (data not shown).

A very similar pattern was observed when the data was expressed as TNF-α, pg/g wet tissue with the reduction in levels, with the low doses of not reaching significance, while those with the intermediate and higher doses did (data not shown).

Treatment with sulfasalazine also significantly reduced the elevated colonic levels of TNF-α as can be seen in FIG. 9. The extent of this reduction in TNF-α levels by sulfasalazine was not significantly different from that brought about by the intermediate or higher dose of CCI-7308 (data not shown).

As in previous studies, the intra-colonic instillation of TNBS (10 mg) caused a subchronic colitis in the rat. This macroscopic injury in the colon, determined 72 h after challenge, consists of areas of haemorrhagic necrosis, with evidence of tissue inflammation and hyperaemia. In the present study, the degree of macroscopic injury involved a mean of 27% of the measured colonic mucosa, and allows sensitive detection of any preventative activity on the lesion development.

Oral administration of the compound CCI-7308 in a twice a day regimen commencing one day prior to TNBS challenge, caused a significant dose-dependent fall in the extent of macroscopically assessed TNBS-induced colonic damage. The findings suggest that the intermediate dose of CCI-7308 of 20 mg/kg/day in divided doses is probably close to the maximal effect, with the higher dose of 100 mg/kg/day producing only a comparable degree of inhibition of lesion area. There was no evidence of a bell-shaped dose response curve within the dose range studied with this compound. Thus this agent may provide a broad therapeutic window for its effective dose-range.

The macroscopic injury provoked by TNBS was accompanied by a substantial increase in the levels of MPO in the colon, which is an index of leukocyte infiltration into the inflamed tissue (Morris et al, 1989; Reuter et al, 1996; Kiss et al, 1997), and reached levels comparable to those reported in the previous study for Nuada (Whittle and Varga, 2004). As with the macroscopic injury, CCI-7308 caused significant and dose-dependent reduction in colonic MPO activity. Interestingly, a significant reduction in MPO was also observed with the lower dose of CCI-7308 that did not significantly reduce the macroscopic lesions. This could reflect the differences of the statistical variances within the data for each of the parameters from these two groups. Whether this finding could also indicate a primary action of CCI-7308 at these lower doses on acute neutrophils influx into the inflammatory site is unknown and would require further investigation.

The elevated weight of the colonic segments following TNBS challenge as an indirect index of oedema, was also dose-dependently reduced by the daily treatment with CCI-7308, a significant effect being observed at the intermediate and higher dose. This may reflect actions of this agent at such doses on the generalised inflammatory response in the tissue, with both white-cell infiltration and oedema being attenuated at these doses.

The fall in body weight that followed the challenge with TNBS was attenuated by CCI-7308 in a dose-dependent manner, with the intermediate and higher doses preventing the fall in body weight on all 3 days post-challenge.

In the present study sulfasalazine at the dose of 50 mg/kg/day significantly reduced the degree of colonic injury and reduced the elevated MPO levels in the colonic tissue. As can be seen from the data, in general, the profile of activity of CCI-7038 on both parameters was similar to that of sulfasalazine, although lower doses of CCI-7308 were effective. Preliminary indication of relative potency from a comparison of the respective molecular weights, would suggest that CCI-7308 is some 2.5 times as active as sulfasalazine in reducing macroscopic injury.

The clinical dose for the 500 mg tablets of the marketed form, Salazopyrin™, is 2-4 tablets×4 times a day for the treatment of active disease in IBD. Based on an average body weight of 75 kg, and the dose range of 4-8 g/day; the lower dose is thus 53 mg/kg/day, while the paediatric doses are given as 40-60 mg/kg/day for acute flare-up. Although pharmacokinetic differences between rat and humans would have to take into account, the effective dose level of sulfasalazine used in the rat in the current study of 50 mg/kg/day, is thus within the range used in the therapeutic control of IBD. This suggests that this model can be predictive of the therapeutic effect of novel agents in colitis.

The elevated colonic levels of TNF-α following challenge with TNBS, a known endogenous mediator of colitis and a good biomarker of disease activity, was significantly reduced by sulfasalazine, as reported previously by others (Ameho et al, 1997; Ribbons et al, 1997; Sykes et al, 1999; Sun et al, 2001; Ten Hove et al, 2001; Villegas et al, 2003). Moreover, in the present work, CCI-7308 significantly reduced the TNF-α levels in a dose-dependent manner, with the intermediate and higher doses reaching significance. The degree of inhibition of the TNF-α levels by CCI-7308 was comparable to that produced by sulfasalazine.

The pre-treatment of rats with a single intravenous dose of infliximab (Remicade™), a therapeutic protein targeting TNF-α, was found to reduce the macroscopic injury, colonic MPO and TNF-α levels observed 8 days after TNBS challenge (Woodruff et al, 2003). The degree of inhibition with infliximab may be comparable to the range to that seen with CCI-7308 at the intermediate and higher doses in the current work.

This study indicates that CCI-7038, given by oral gavage twice daily commencing 1 day prior to challenge, dose-dependently reduces the degree of tissue injury therapeutic activity in this 3-day rat model of colitis, reducing macroscopic colonic injury at both the intermediate and higher doses employed (20 and 100 mg/kg/day). The biomarkers of colonic inflammation, MPO and also TNF-α, the latter being a known inflammatory mediator involved in colitis, were also dose-dependently reduced in the inflamed tissue by these doses of CCI-7308. Overall, the findings suggest that a dose of CCI-7308 of 20 mg/kg/day in divided doses is close to the maximal effective dose in this model. This profile of actions of CCI-7038 were comparable to those seen with sulfasalazine, an agent used widely in the clinic in the therapy of IBD, and estimates of relative potency suggest a 2.5-fold greater activity with CCI-7308 on macroscopic injury, and probably the other biomarkers.

### Example 43

### Comparison of the Effects of CCI-7506, CCI-7507, Sulfasalazine or Infliximab in a Rat Model of Chronic Colitis Provoked by Trinitrobenzene Sulphonic Acid (TNBS) Over 14 Days

In the present example, the effects of the following compounds were evaluated in the TNBS model.

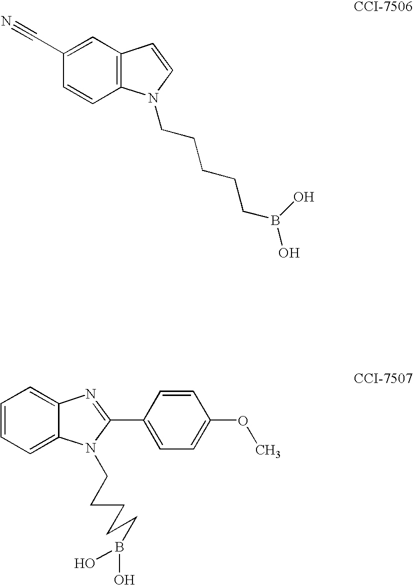

In the chronic model of colitis, assessment of the colonic inflammation is made 14 days or longer, after the intracolonic challenge with TNBS (Boughton-Smith et al, 1988a, 1988b; Wallace et al, 1989; Rachmilewitz et al, 1989; Wallace and Keenan, 1990; Ameho et al, 1987; Sans et al, 1999; Sun et al, 2001; Maric et al, 2003; Moreels et al, 2004; Gonzalez et al, 2004). This model allows treatment with experimental agents to commence following the establishment of the colonic injury, typically 24 hours after the TNBS challenge (Galvez et al, 2000, Villegas, 2003, Gonzalez et al, 2004). The model should therefore identify the ability of the experimental compounds to accelerate the diminution of the inflammatory response and to promote healing of the colonic lesions. This model thus has relevance additional to the acute model, as the clinical correlate is the therapeutic intervention in patients with existing IBD not in remission or with flare-up, to reduce the crisis. This contrasts with the acute TNBS model where the compounds are administered one or two days prior to challenge, the clinical correlate being the use of prophylactic therapy to prevent flare-up and maintain remission in IBD patients.

In this current study, the low intracolonic challenge concentration of TNBS used in the acute studies was also used for the chronic study over a 14 day period. This concentration and timing was based on the findings from pilot studies where a range of concentrations of TNBS and treatment conditions were evaluated over a 14 day period. The dose of TNBS (10 mg) in rats starved for 12 hours, proved to yield significant colonic injury after 14 days, not dissimilar from that with the high dose of 30 mg, yet substantially reduced the high incidence of mortality and diarrhoea observed with the higher dose in the model and as reported by others with this higher dose over sub-chronic periods (Woodruff et al, 2003).

The methods and protocol used were substantially similar to those in Example 39-40, with some differences described below.

TNBS Challenge. Male Wistar rats (average body weight, 210 g) were randomised into groups before commencement of the study. In all groups, including the non-challenged and non-treated absolute control group, food was withdrawn for 12 h before TNBS administration (i.e. overnight on Day −1), but the rats were allowed free access to drinking water.

On the morning of the day of challenge (Day 0, between 9.00 and 1 1.00 a.m.), the rats were transiently anaesthetised with ether and the TNBS solution (10 mg in 0.25 ml of 50% ethanol) was instilled approximately 6-8 cm into the colon using a soft plastic catheter inserted in the rat rectum. The rats were allowed to recover with free access to food and drinking water. At the end of the experiment, on the morning of Day 14, between 9.00 and 11.00), the colon was dissected, and the distal 8 cm photographed and immediately processed or stored appropriately for subsequent analyses.

The following primary parameters were measured in the study: macroscopic scoring of distal 8 cm of colon; myeloperoxidase levels in segments of distal 8 cm of colon; TNF-α levels in segments of distal 8 cm of colon.

In addition, the weight of the standard colonic segment was assessed as an indirect and non-specific marker of oedema. The body weight of the animals was also determined each evening of the study, starting on Day-1, and also on the morning of Day 14. The data is shown graphically as the % change from the weight on Day-1, prior to challenge.

Treatments. The TNBS challenged groups for study were: (a) Vehicle control 0.5% carboxy methyl cellulose (CMC) p.o., twice daily from Day; (b) CCI-7506 25 mg/kg, p.o., twice daily from Day 1 (50 mg/kg/day total); (c) CCI-7506 50 mg/kg, p.o., twice daily from Day 1 (100 mg/kg/day total); (d) CCI-7507 12.5 mg/kg, p.o., twice daily from Day 1 (25 mg/kg/day total); (e) CCI-7507 25 mg/kg, p.o., twice daily from Day 1(50 mg/kg/day total); (f) Sulfasalazine 25 mg/kg, p.o., twice daily from Day 1 (50 mg/kg/day total); (g) Infliximab 3 mg/kg, single slow i.v. injection, on Day 1 and Day7.

The experimental compounds that were administered orally were given twice a day from Day 1, i.e. 24 h following TNBS administration, for the remainder of the 14 day experimental period. Infliximab was administered by slow i.v. injection of Day 1 and on Day 7. This latter group of rats was also administered 0.5% w/v CMC (0.5 ml p.o.) twice a day from Day 1. Dosing was performed once in the morning (between 9:00 and 11:00) and once in the late afternoon (between 18:00 and 21:00). In addition, a group of rats that were non-treated and non-challenged, were also evaluated for base-line measurements.

Preparation and Dose of Infliximab. The dose of infliximab (Remicade; Centecor-Schering Plough) of 3 mg/kg as a slow intravenous injection used in this protocol, is comparable to that used in the clinical studies on IBD. This does has also been used in the experimental setting in vivo to attenuate the response to TNF-α in acute or chronic inflammatory conditions in the rat (Kulmatycki et al, 2001; Woodruff et al, 2003) and in our own in-house studies in the acute TNBS model. Infliximab was dissolved in the supplied diluent, sterile saline for injection, immediately prior to use, as indicated in the technical documents supplied with the material.

Macrocopic Injury. Macroscopic injury was performed as described above. In addition to the quantitative measurement of area of damage, the degree of colonic damage was also assessed in a randomised blinded fashion using a Damage Score, utilizing a 1-5 scale than has been adapted from that used previously (Boughton-Smith et al, 1988a):0═No Damage; 1=One region of localized inflammation or thickening (No ulcers); 2=Linear ulceration, but no significant inflammation; 3=Linear ulceration with inflammation at one site; 4=Two or more sites of ulceration and/or inflammation (Ulcers present in at least one site); 5=Two or more sites of ulceration and inflammation or one major site of ulceration and inflammation extending >1 cm along the length of the colon.

Results. In the following figures the labels have the following meaning. TNBS=2,4,6-Trinitrobenzenesulfonic acid solution (10 mg); CMC=carboxy methylcellulose; Abs. control=non-challenged and non-treated;

CMC=TNBS+0.5% CMC (b.i.d., 0.5 ml/rat p.o.)

CCI-7506-50 mg=TNBS+CCI-7506 treated group (50 mg/kg/day p.o. total dose)

CCI-7506-100 mg=TNBS+CCI-7506 treated group (100 mg/kg/day p.o. total dose)

CCI-7507-25 mg=TNBS+CCI-7507 treated group (25 mg/kg/day p.o. total dose)

CCI-7507-50 mg=TNBS+CCI-7507 treated group (50 mg/kg/day p.o. total dose)

SASP=TNBS+Sulfasalazine treated group (50 mg/kg/day p.o. total dose)

Infliximab=TNBS+Infliximab (3 mg/kg i.v. on Day 1 and Day 7)+0.5% CMC (b.i.d., 0.5 ml/rat p.o.)

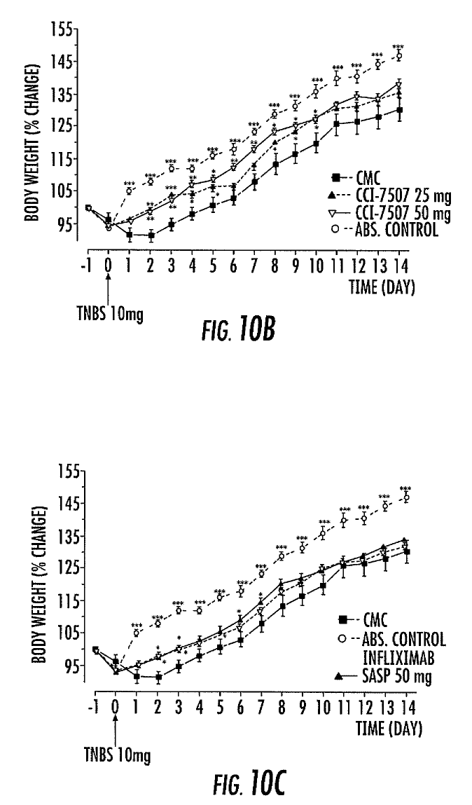

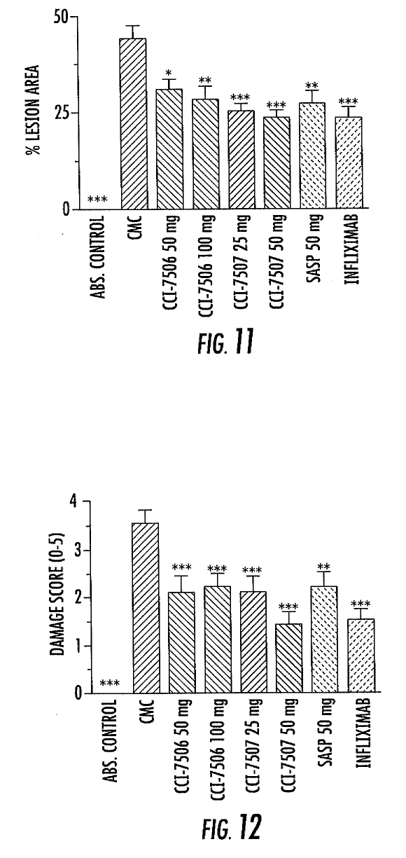

Body Weight. FIGS. 10A-10C show the effects of CCI-7506 (50 and 100 mg/kg/day p.o.), CCI-7507 (25 and 50 mg/kg/day p.o.), sulfasalazine (50 mg/kg/day p.o.) or infliximab (3 mg/kg i.v on Day 1 and 7) on body weight over 14 days, expressed a % change of the body weight at Day −1, prior to TNBS challenge on Day 0. The orally administered compounds were given in divided doses in a twice a day dosing schedule, commencing in the morning of Day 1, i.e. 24 h after TNBS challenge. All groups including the non-challenged, non-treated absolute control group were starved for 12 h overnight on Day −1. Results are expressed as mean±S.E.M., n=11-15 for the test groups and n=6 for the absolute control group; statistical significance is shown as *P<0.05, **P<0.01, ***P<0.001 compared with the challenged control CMC group.

In the non-challenged and non-treated absolute control group, there was an apparent small fall in body weight of the rats on Day 0 from its value on Day-1, presumably as a consequence of the 12 h period of food removal overnight. From Day 1 onwards, the body weight in this group progressively increased and was then significantly different for the CMC challenged group at all time points up to 14 days (FIGS. 1A-1C). As in the absolute control group, there was an apparent initial small transient fall in body weight on Day 0 compared with Day-1 in all of the TNBS-challenged groups (FIGS. 1A-1C). There was no statistically significant difference between any of the groups on Day 0 (data not shown)

Following challenge with TNBS, there was a further fall in body weight observed in the CMC-challenged control group on Day 1 (P<0.001), reaching its peak on Day 2 post-challenge. The body weight then recovered progressively during the 14 day period, and by Day 4, was no longer significantly different from the value at Day-1. There was no significant difference in the % change in body weights between any of the challenged groups on Day 1 (data not shown).

Treatment with CCI-7506 (50 and 100 mg/kg/day, administered orally in divided doses of 25 and 50 mg/kg b.i.d respectively) commencing 24 h after TNBS challenge on Day 1, caused an attenuation of this fall in body weight, as shown in FIG. 10A. The change in body weights of the lower dose of CCI-7506 was significantly different from those in the challenged CMC control group on Days 2 to 10 and also on Days 13 and 14. With the higher dose, the changes in the fall in body weight were likewise attenuated, and were significantly different from the CMC group on Days 2, 3 and 4 (P<0.05) as shown in FIG. 1C.

CCI-7507 (25 and 50 mg/kg/day, administered orally in divided doses of 12.5 and 25 mg/kg b.i.d) also attenuated the TNBS-induced fall in body weight (FIG. 1B). With the lower dose, the change in body weight was significantly different from that in the CMC challenged group (P<0.05) on Days 2, 3, 4, 8, 9 and 10 post-challenge. With the higher dose of CCI-7507 (50 mg/kg/day), the change in body weight was significantly different form the CMC group on all days from Day 2 to 10.

Treatment with sulfasalazine (50 mg/kg/day administered orally in divided doses of 25 mg/kg b.i.d), also attenuated the body weight loss following TNBS challenge (FIG. 10C), which reached statistical significance compared with the CMC challenged group on at Day 2, 3, 6 and 7.

Intravenous injection of infliximab (3 mg/kg on Day 1 and on Day 7) attenuated the fall in body weight following TNBS, with the change compared to the CMC challenged group being significant on Day 2 and 3 (FIG. 10C).

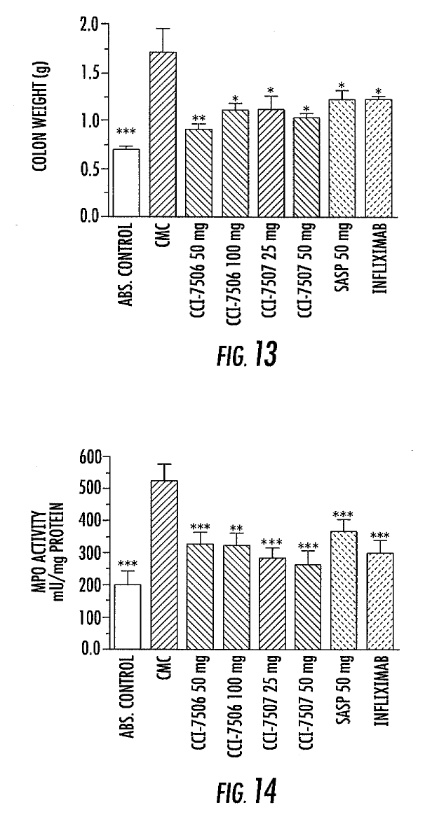

Macroscopic Colonic Injury. Area of Damage. FIG. 11 shows the effects of CCl-7506 (50 and 100 mg/kg/day p.o.), CCI-7507 (25 and 50 mg/kg/day p.o.), sulfasalazine (50 mg/kg/day p.o.) or infliximab (3 mg/kg i.v on Day 1 and 7) on macroscopic injury in the colon, determined 14 days after TNBS challenge, as assessed as the colonic lesion area, % of the total area measured. The orally administered compounds were given in divided doses in a twice a day dosing schedule, commencing on Day 1, i.e. 24 h after TNBS challenge. Results are expressed as mean±S.E.M., n=1 1-15 for the test groups and n=6 for the absolute control group; statistical significance is shown as *P<0.05, **P<0.01, ***P<0.001 compared with the challenged control CMC group.

In the group challenged with TNBS (10 mg) on Day 0, followed by the vehicle 0.5% CMC vehicle p.o. twice a day, commencing from Day 1, i.e. 24 h after challenge, the area of injury determined after 14 days involved 44±4% (n=14) and of the total colonic area of the segment studied, determined by computerized planimetric measurement (FIG. 11). In a pilot study, administration of the vehicle 0.5% CMC vehicle p.o., twice a day for Day 1, had no significant effect on the area of colonic injury induced by TNBS (10 mg) after 14 days. There was no detectable macroscopic injury in the colons in the non-challenged, non-treated absolute control group of rats (FIG. 11).

Treatment with CCI-7506 (50 and 100 mg/kg/day administered orally in divided doses), commencing 24 h after the TNBS challenge, caused a dose-dependent reduction in the area of colonic injury observed on Day 14 (FIG. 11). This reduction in TNBS-induced colonic damage was statistically significant for both doses (P<0.01; see Appendix I, Table 10 for full tabular data). Treatment with CCI-7507 (25 and 50 mg/kg/day administered orally in divided doses) commencing 24 h after challenge likewise caused a dose-dependent reduction in the area of colonic injury observed at Day 14 (FIG. 11). This reduction in TNBS-induced colonic damage was statistically significant for both doses (P<0.001; data not shown). The effect of CCI-7507 at the dose of 50 mg/kg/day was significantly (P<0.05) greater than that observed with CCI-7506 at that same dose (data not shown). Treatment with sulfasalazine (50 mg/kg/day administered orally in divided doses) commencing 24 h after challenge, significantly (P<0.001) reduced the extent of macroscopic injury seen at Day 14 after challenge, as shown in FIG. 11. This effect was not significantly different from that observed with any of the other active treatment groups. Intravenous injection of infliximab (3 mg/kg on Day 1 and on Day 7 after challenge) significantly attenuated the area of injury following TNBS, observed on Day 14 (FIG. 11). This effect was not significantly greater than that observed with CCI-7506, CCI-7507 or sulfasalazine (FIG. 12).

Macroscopic Damage Score. In addition to the area of visible injury, the degree of macroscopic colonic injury was assessed using as Damage Score (scale 1-5), as shown in FIG. 12. Results are expressed as mean±S.E.M., n=11-15 for the test groups and n=6 for the absolute control group; statistical significance is shown as **P<0.01, ***P<0.001 compared with the challenged control CMC group.

As can be seen, the scores in the treatment groups closely followed the profile of that determined by the quantitative measurement of area of damage. Thus, CCI-7506 (50 and 100 mg/kg/day) and CCI-7507(25 and 50 mg/kg/day) at both doses reduced the damage score observed in the colons at Day 14, as did both sulfasalazine (50 mg/kg/day) and infliximab (3 mg/kg on Day 1 and 7), as shown in FIG. 12.

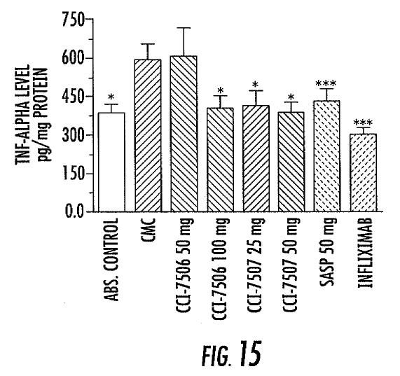

Colon Weight. As an indirect index of inflammatory oedema in the colonic tissue, the weight of the standard colonic segments was determined at the end of the study. The colonic weight in the CMC challenged group was significant higher at Day 14 than that in the non-challenged, non-treated group (FIG. 13). Results are expressed as mean±S.E.M., n=11-15 for the test groups and n=6 for the absolute control group; statistical significance is shown as *P<0.05, **P<0.01, ***P<0.001 compared with the challenged control CMC group.

Treatment with CCI-7506 at both 50 and 100 mg/kg/day caused a significant reduction in the colon weight determined on Day 14 (FIG. 13). CCI-7507 (25 and 50 mg/kg/day) also significantly reduced the colonic weight of the standard segment at both doses compared with that in the CMC group (FIG. 13). A significant (P<0.05) reduction in colon weight was also observed in the sulfasalazine group and in the infliximab group (FIG. 13).

Colonic MPO Levels. The level of MPO activity in the colonic tissue from the TNBS-challenged group determined after 14 days was 521±56 mU/mg protein, being significantly different from that determined in the absolute control group, as shown in FIG. 14. Results are expressed as mean±S.E.M., n=11-15 for the test groups and n=6 for the absolute control group; statistical significance is shown as **P<0.01, ***P<0.001 compared with the challenged control CMC group.

Treatment with CCI-7506 (50 and 100 mg/kg/day) caused a significant fall in the elevated MPO activity determined at 14 days, at both dose levels, as shown in FIG. 14. Treatment with CCI-7507 (25 and 50 mg/kg/day) also caused a significant and dose-dependent fall in the elevated MPO activity at both dose levels, as shown in FIG. 14. Treatment with sulfasalazine (50 mg/kg/day) significantly (P<0.001) reduced the elevated colonic levels of MPO as can be seen in FIG. 14. This effect was not significantly different from that observed with any of the other active treatment groups (data not shown). Intravenous injection of infliximab (3 mg/kg on Day 1 and on Day 7 after challenge) significantly attenuated the increase in MPO, following TNBS, observed on Day 14 (FIG. 14). This effect was not significantly greater than that observed with the lower or higher doses of either CCI-7506 or CCI-7507, or that with sulfasalazine (FIG. 14).

Colonic TNF-α Levels. Challenge with TNBS significantly elevated the levels of TNF-α in the colonic tissue determined after 14 days compared with the absolute control group(FIG. 15). Results are expressed as mean±S.E.M., n=11-15 for the test groups and n=6 for the absolute control group; statistical significance is shown as *P<0.05, ***P<0.001 compared with the challenged control CMC group. The level of TNF-α in the colonic tissue from TNBS-challenged rats, determined after 14 days after challenge was 589±66 pg/mg protein (FIG. 15).

Treatment with CCI-7506 dose-dependently reduced the level of TNF-α in the colonic tissues (FIG. 15). Thus, whereas CCI-7506 (50 mg/kg/day) had no significant effect on the colonic TNF-α levels, the higher dose of 100 mg/kg/day did achieve significance (FIG. 15). Administration of either dose of CCI-7507 (25 and 50 mg/kg/day) caused a significant reduction in the elevated levels of TNF-α determined at Day 14, as shown in FIG. 15. Treatment with sulfasalazine also significantly reduced the elevated colonic levels of TNF-α observed on Day 14 following TNBS challenge, as can be seen in FIG. 15. Intravenous injection of infliximab (3 mg/kg on Day 1 and on Day 7 after challenge) significantly attenuated the increase in TNF-α levels FIG. 15. This effect was not significantly greater than that observed with either of the doses of CCI-7507, but was significantly greater than that achieved with the lower dose of CCI-7506 (50 mg/kg/day) or with sulfasalazine.

This Example indicates that both CCI-7506 (50 and 100 mg/kg/day) and CCl-7507 (25 and 50 mg/kg/day) given by oral gavage twice daily commencing 1 day following challenge, dose-dependently reduce the degree of tissue injury a 14 day rat model of colitis, reducing macroscopic colonic injury at the doses of both agents employed. The biomarkers of colonic inflammation, colon weight and colonic MPO levels, along with TNF-α, the latter being an inflammatory mediator involved in colitis, were also reduced in the inflamed tissue by these doses of CCI-7506 and CCI-7507. This profile of pharmacological actions of CCI-7506 and CCI-7507 were comparable to those seen with sulfasalazine, an agent used widely in the clinic in the therapy of IBD, and preliminary estimates could suggest a greater activity of CCI-7507 on macroscopic injury, and the other key biomarkers in the chronic study. Moreover, the profile of activity with the compounds was also comparable to those of infliximab.

### Example 44

### Anti-Inflammatory Activity of a Representative Presently Disclosed Compound in a Mouse Ear Edema Model

The anti-inflammatory activity of 5-(2-(4-Methoxyphenyl)-1H-benzo[d]imidazol-1-yl)pentylboronic acid topically applied in a dose-response model of arachidonic acid-induced ear edema was assessed.

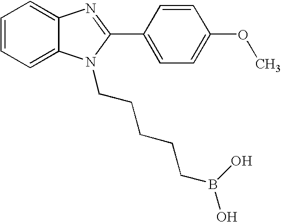

### Summary of Procedures:

This example was carried out in male BALB/c mice. 42 BALB/c mice (Harlan Sprague-Dawley, Inc., male, PO # 452036, R #2449, 5-6 weeks) were received, individually examined, and housed in four cages of ten mice each and one cage containing two mice. Each animal was in apparent good health: no clinical signs of disease or distress. The animals were placed in quarantine with daily inspections.

The animals were examined and appeared to be free of clinical symptoms of disease or distress. The mice were released to routine maintenance. No deaths were recorded during the quarantine period.

An aliquot of a representative presently disclosed compound was stored in an amber glass vial and stored at 25° C. Because the material was not soluble in acetone, 69.9 mg of 5-(2-(4-Methoxyphenyl)-1H-benzo[d]imidazol-1-yl)pentylboronic acid was dissolved in 1.389-mL Graves Grain alcohol (190 proof) to prepare a 5% solution. 0.1 mL of this solution was diluted into 0.9 mL 190 proof alcohol to prepare a 0.5% solution.

30 mg indomethacin (Sigma Cat. I-7378, lot 60K0745) was dissolved in 5 ml 0.1 M NaHCO₃ to prepare a 6 mg/mL solution.

48.9 μL arachidonic acid (Sigma Cat. A-9673. lot 057K₁₆₂₀) was dissolved in 450 μL 190 proof alcohol to prepare a 100 mg/mL solution.

The mice were numbered and weighed. The mice in Groups 1, 3, 4 were topically treated on both sides of both ears with either 25 μL 190 proof alcohol, 5% 5-(2-(4-Methoxyphenyl)-1H-benzo[d]imidazol-1-yl)pentylboronic acid, or 0.5% 5-(2-(4-Methoxyphenyl)-1H-benzo[d]imidazol-1-yl)pentylboronic acid. The mice in Group 2 were injected intraperitoneally with 5 mL/kg indomethacin (30 mg/kg). Thirty minutes after 190 proof alcohol, indomethacin, or 5-(2-(4-Methoxyphenyl)-1H-benzo[d]imidazol-1-yl)pentylboronic acid application/administration, 5 μL of the arachidonic acid solution was applied to the dorsal and ventral sides of the right ear. The contralateral ear was treated with 190 proof alcohol. One hour after alcohol/arachidonic acid application, the mice were euthanized, the ears removed and the ear weights recorded.

Results:

Arachidonic Acid Challenge:

In response to a topical application of arachidonic acid to the right ears of mice, a 3-fold increase in ear weight was recorded one hour later. The quantative difference between the arachidonic acid- and the vehicle-treated ears was 50.2±4.2 mg.

Prophylactic Treatment with Indomethacin:

Intraperitoneal injection with 30 mg/kg indomethacin thirty minutes prior to arachidonic acid challenge resulted in a significant (p=5×10⁻¹⁴) 75% inhibition of the irritant-induced ear edema.

Prophylactic Treatment with 5-(2-(4-Methoxyphenyl)-1H-benzo[d]imidazol-1-yl)pentylboronic acid:

Prophylactic topical treatment with 5-(2-(4-Methoxyphenyl)-1H-benzo[d]imidazol-1-yl)pentylboronic acid, thirty minutes prior to challenge with arachidonic acid resulted in a dose-dependent inhibition of the response to the irritant. At the highest concentration (5%) a significant (p=2×10⁻⁹) 46% inhibition was observed, whereas at the 0.5% concentration only a 12% (p=0.06) inhibition was measured.

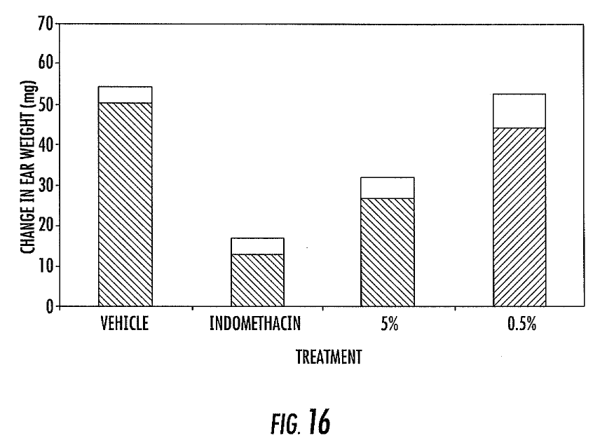

The results are summarized in Tables 27-29. Significance (p-value) was calculated using Student's T-test. The effect of prophylactic topical treatment with a representative presently disclosed compound on arachidonic acid-induced murine ear edema is shown in FIG. 16.

TABLE 27

| | | | Ear Weight (mg) | |
|---|---|---|---|---|
| Mouse | Weight (g) | Treatment | Right (+AA) | Left (−AA) |
| 1 | 21 | Vehicle, topical | 80 | 26 |
| 2 | 21 | | 71 | 23 |
| 3 | 22 | | 71 | 24 |
| 4 | 22 | | 75 | 24 |
| 5 | 20 | | 66 | 22 |
| 6 | 21 | | 90 | 32 |
| 7 | 22 | | 74 | 24 |
| 8 | 19 | | 67 | 21 |
| 9 | 20 | | 73 | 22 |
| 10 | 21 | | 78 | 25 |
| 1 | 21 | Indomethacin | 44 | 23 |
| 2 | 20 | 30 mg/kg, i.p. | 32 | 20 |
| 3 | 21 | | 33 | 21 |
| 4 | 19 | | 35 | 22 |
| 5 | 19 | | 32 | 23 |
| 6 | 19 | | 31 | 22 |
| 7 | 22 | | 34 | 24 |
| 8 | 22 | | 32 | 22 |
| 9 | 22 | | 38 | 24 |
| 10 | 23 | | 43 | 26 |
| 1 | 21 | 5-(2-(4-Methoxy- | 50 | 23 |
| | | phenyl)-1H- | | |
| | | benzo[d]imidazol-1- | | |
| | | yl)pentylboronic acid | | |
| 2 | 20 | 5%, topical | 48 | 24 |
| 3 | 21 | | 57 | 22 |
| 4 | 19 | | 42 | 23 |
| 5 | 21 | | 55 | 22 |
| 6 | 21 | | 51 | 20 |
| 7 | 21 | | 53 | 24 |
| 8 | 21 | | 49 | 23 |
| 9 | 21 | | 44 | 22 |
| 10 | 21 | | 44 | 21 |

TABLE 28

| | | | Ear Weight (mg) | |
|---|---|---|---|---|
| Mouse | Weight (g) | Treatment | Right (+AA) | Left (−AA) |
| 1 | 20 | 5-(2-(4-Methoxy- | 64 | 20 |
| | | phenyl)-1H- | | |
| | | benzo[d]imidazol-1- | | |
| | | yl)pentylboronic | | |
| | | acid | | |
| 2 | 24 | 0.5%, topical | 76 | 22 |
| 3 | 20 | | 63 | 20 |
| 4 | 21 | | 55 | 20 |
| 5 | 20 | | 82 | 34 |
| 6 | 21 | | 78 | 23 |
| 7 | 20 | | 50 | 20 |
| 8 | 20 | | 62 | 22 |
| 9 | 22 | | 77 | 24 |
| 10 | 20 | | 60 | 21 |

TABLE 29

| | Change in Ear Weight (mg) | | | |
|---|---|---|---|---|
| Treatment | Average | SD | p-value | % Inhibition |
| Vehicle | 50.2 | 4.2 | N/A | N/A |
| Indomethacin, 30 mg/kg, ip | 12.7 | 3.8 | 5 × 10⁻¹⁴ | 74.7 |
| 5% 5-(2-(4-Methoxyphenyl)- | 26.9 | 5.1 | 2 × 10⁻⁹ | 46.4 |
| 1H-benzo[d]imidazol-1- | | | | |
| yl)pentylboronic acid | | | | |
| 0.5% 5-(2-(4- | 44.1 | 8.4 | 0.06 | 12.2 |
| Methoxyphenyl)- | | | | |
| 1H-benzo[d]imidazol-1- | | | | |
| yl)pentylboronic acid | | | | |

All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

The foregoing is illustrative of the present invention, and is not to be construed as limiting thereof. The invention is defined by the following claims, with equivalents of the claims to be included therein. Although the foregoing subject matter has been described in some detail by way of illustration and example for purposes of clarity of understanding, it will be understood by those skilled in the art that certain changes and modifications can be practiced within the scope of the appended claims.

That which is claimed is:

1. A method of inhibiting an inflammatory cytokine in a subject in need thereof, the method comprising administering to the subject a compound selected from the group consisting of:
(a) a compound of Formula I or Formula II 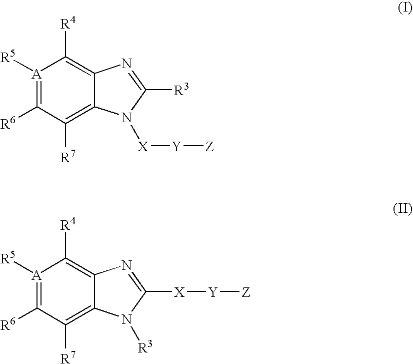
(b) a compound of Formula III, Formula IV, or Formula V: 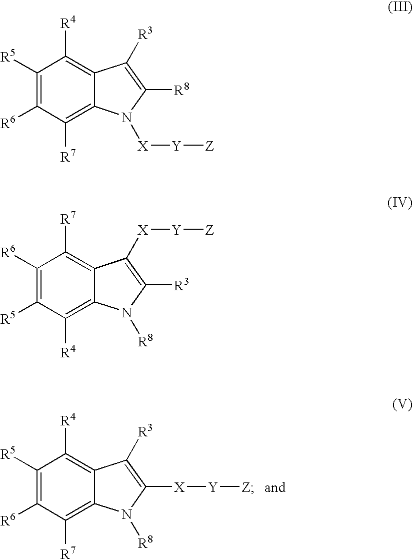
(c) a compound of Formula VI: 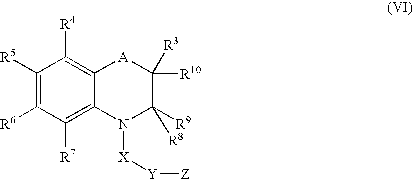
wherein:
A is N or C in compounds of Formula I and II, subject to the proviso that R⁵ is absent when A is N;
A is S, O, SO₂ or NR in compounds of Formula VI;
X is —C(O)—, —S(O)₂—, or a covalent bond;
Y is alkyl, alkenyl, cycloalkyl, alkylcycloalkyl, alkylcycloalkylalkyl, alkyloxyalkyl, aryl, alkylaryl, alkylarylalkyl, arylalkyl, cycloalkylalkyl, alkylheterocycle, heterocyclealkyl, alkylheterocyclealkyl, heterocycle, aminoalkyl, oxyalkyl, aminoaryl, oxyaryl;
Z is selected from the group consisting of —B(OR¹)OR², —CON(R¹)OR², and —N(OR¹)COR²;
R¹ and R² are each independently H, loweralkyl, or together form C₂-C₄ alkylene; and
R³, R⁴, R⁵, R⁶, and R⁷ and, if present, R, R⁸, R⁹, and R¹⁰ are each independently selected from the group consisting of: H, halo, loweralkyl, haloloweralkyl, haloloweralkoxy, loweralkoxy, hydroxy, loweralkoxycarbo, cycloalkyl, alkylcycloalkyl, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, nitro; arylalkyloxy, cycloalkyloxy, cycloalkylalkoxy, cycloalkylamino, urea, cycloalkylalkylamino, cycloalkyl, alkylcycloalkyl, hydroxyamino, alkoxyacylamino, and arylthio;
and 5- or 6-membered organic rings containing 0 to 4 heteroatoms selected from the group consisting of N, O and S, which rings may be unsubstituted or substituted from 1 to 4 times with halo, loweralkyl, haloloweralkyl, haloloweralkyloxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, nitro; and oxoheterocyclic groups;
or R⁸ and R⁹, if present, together are ═O or ═S;
or a pharmaceutically acceptable salt or prodrug thereof;
in an amount effective to inhibit the inflammatory cytokine.

2. The method of claim 1, wherein the compound is a compound of Formula I or Formula II and the compound is selected from the group consisting of:
4-(2-(Trifluoromethyl)-1H-benzo[d]imidazol-1-yl)butylboronic acid;
5-(2-(Thiazol-4-yl)-1H-benzo[d]imidazol-1-yl)pentylboronic acid;
5-(5,6-dimethyl-1H-benzo[d]imidazol-1-yl)pentylboronic acid;
5-(1H-imidazo[4,5-c]pyridin-1-yl)pentylboronic acid;
5-(2-(4-Methoxyphenyl)-1H-benzo[d]imidazol-1-yl)pentylboronic acid;
5-(2-(3-Fluoro-4-methoxyphenyl)-1H-benzo[d]imidazol-1-yl)pentylboronic acid;
5-(5-cyano-2-(4-methoxyphenyl)-1H-benzo[d]imidazol-1-yl)pentylboronic acid;
5-(6-cyano-2-(4-methoxyphenyl)-1H-benzo[d]imidazol-1-yl)pentylboronic acid;
and pharmaceutically acceptable salts and prodrugs thereof.

3. The method of claim 1, wherein the compound is a compound of Formula III, IV, or V and the compound is selected from the group consisting of:
5-(5-cyano-1H-indol-1-yl)pentylboronic acid;
and pharmaceutically acceptable salts and prodrugs thereof.

4. The method of claim 1, wherein the compound is a compound of Formula VI and the compound is selected from the group consisting of:
5-(6-fluoro-2,3-dihydro-3-oxobenzo[b][1,4]oxazin-4-yl)pentylboronic acid;
5-(2,3-dihydro-3-oxobenzo[b][1,4]thiazin-4-yl)pentylboronic acid;
5-(7-chloro-2,3-dihydro-3-oxobenzo[b][1,4]thiazin-4-yl)pentylboronic acid;
5-(2,3-dihydro-7-nitro-3-oxobenzo[b][1,4]oxazin-4-yl)pentylboronic acid;
5-(2,3-dihydro-3-oxobenzo[b][1,4]oxazin-4-yl)pentylboronic acid;
ethyl 2-(3,4-dihydro-3-oxo-4-(5-pentylboronic acid)-2H-benzo[b][1,4]thiazin-2-yl)acetate;
and pharmaceutically acceptable salts and prodrugs thereof.

5. The method of claim 1, wherein the inflammatory cytokine is tumor necrosis factor alpha.

6. The method of claim 1, wherein the inhibiting of the inflammatory cytokine comprises reducing the production of tumor necrosis factor alpha.

7. A method of inhibiting phosphodiesterase in a subject in need thereof, the method comprising administering to the subject a compound selected from the group consisting of:
(a) a compound of Formula I or Formula II 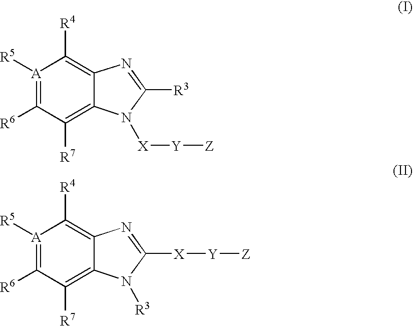
(b) a compound of Formula III, Formula IV, or Formula V: 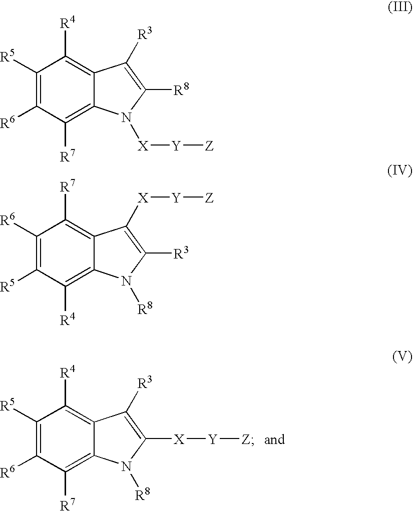
(c) a compound of Formula VI: 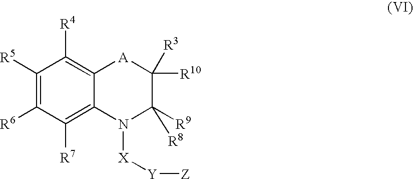
wherein:
A is N or C in compounds of Formula I and II, subject to the proviso that R⁵ is absent when A is N;
A is S, O, SO₂ or NR in compounds of Formula VI;
X is —C(O)—, —S(O)₂—, or a covalent bond;
Y is alkyl, alkenyl, cycloalkyl, alkylcycloalkyl, alkylcycloalkylalkyl, alkyloxyalkyl, aryl, alkylaryl, alkylarylalkyl, arylalkyl, cycloalkylalkyl, alkylheterocycle, heterocyclealkyl, alkylheterocyclealkyl, heterocycle, aminoalkyl, oxyalkyl, aminoaryl, oxyaryl;
Z is selected from the group consisting of —B(OR¹)OR², —CON(R¹)OR², and —N(OR¹)COR²;
R¹ and R² are each independently H, loweralkyl, or together form C2-C4 alkylene; and
R³, R⁴, R⁵, R⁶, and R⁷ and, if present, R, R⁸, R⁹, and R¹⁰ are each independently selected from the group consisting of: H, halo, loweralkyl, haloloweralkyl, haloloweralkoxy, loweralkoxy, hydroxy, loweralkoxycarbo, cycloalkyl, alkylcycloalkyl, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, nitro; arylalkyloxy, cycloalkyloxy, cycloalkylalkoxy, cycloalkylamino, urea, cycloalkylalkylamino, cycloalkyl, alkylcycloalkyl, hydroxyamino, alkoxyacylamino, and arylthio;
and 5- or 6-membered organic rings containing 0 to 4 heteroatoms selected from the group consisting of N, O and S, which rings may be unsubstituted or substituted from 1 to 4 times with halo, loweralkyl, haloloweralkyl, haloloweralkyloxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, nitro; and oxoheterocyclic groups;
or R⁸ and R⁹, if present, together are ═O or ═S;
or a pharmaceutically acceptable salt or prodrug thereof, in an amount effective to inhibit phosphodiesterase.

8. The method of claim 7, wherein the compound is a compound of Formula I or Formula II and the compound is selected from the group consisting of:
4-(2-(Trifluoromethyl)-1H-benzo[d]imidazol-1-yl)butylboronic acid;
5-(2-(Thiazol-4-yl)-1H-benzo[d]imidazol-1-yl)pentylboronic acid;
5-(5,6-dimethyl-1H-benzo[d]imidazol-1-yl)pentylboronic acid;
5-(1H-imidazo[4,5-c]pyridin-1-yl)pentylboronic acid;
5-(2-(4-Methoxyphenyl)-1H-benzo[d]imidazol-1-yl)pentylboronic acid;
5-(2-(3-Fluoro-4-methoxyphenyl)-1H-benzo[d]imidazol-1-yl)pentylboronic acid;
5-(5-cyano-2-(4-methoxyphenyl)-1H-benzo[d]imidazol-1-yl)pentylboronic acid;
5-(6-cyano-2-(4-methoxyphenyl)-1H-benzo[d]imidazol-1-yl)pentylboronic acid;
and pharmaceutically acceptable salts and prodrugs thereof.

9. The method of claim 7, wherein the compound is a compound of Formula III, IV, or V and the compound is selected from the group consisting of:
5-(5-cyano-1H-indol-1-yl)pentylboronic acid;
and pharmaceutically acceptable salts and prodrugs thereof.

10. The method of claim 7, wherein the compound is a compound of Formula VI and the compound is selected from the group consisting of:
5-(6-fluoro-2,3-dihydro-3-oxobenzo[b][1,4]oxazin-4-yl)pentylboronic acid;
5-(2,3-dihydro-3-oxobenzo[b][1,4]thiazin-4-yl)pentylboronic acid;
5-(7-chloro-2,3-dihydro-3-oxobenzo[b][1,4]thiazin-4-yl)pentylboronic acid;
5-(2,3-dihydro-7-nitro-3-oxobenzo[b][1,4]oxazin-4-yl)pentylboronic acid;
5-(2,3-dihydro-3-oxobenzo[b][1,4]oxazin-4-yl)pentylboronic acid;
ethyl 2-(3,4-dihydro-3-oxo-4-(5-pentylboronic acid)-2H-benzo[b][1,4]thiazin-2-yl)acetate;
and pharmaceutically acceptable salts and prodrugs thereof.

11. The method of claim 7, wherein the phosphodiesterase (PDE) is selected from the group consisting of PDE II, PDE III, PDE IV, PDE V and combinations thereof.

12. A method of treating an inflammatory disease in a subject in need thereof, the method comprising administering to the subject a compound selected from the group consisting of:
(a) a compound of Formula I or Formula II 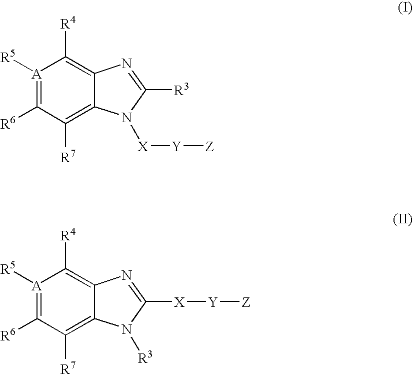
(b) a compound of Formula III, Formula IV, or Formula V: 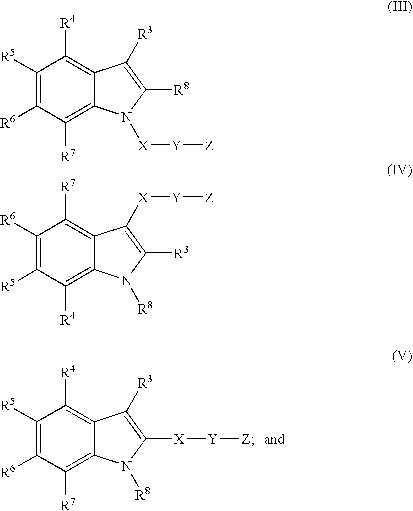
(c) a compound of Formula VI: 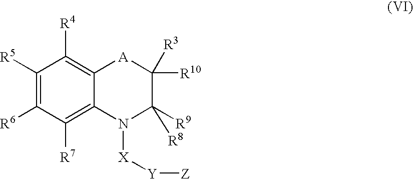
wherein:
A is N or C in compounds of Formula I and II, subject to the proviso that R⁵ is absent when A is N;
A is S, O, SO₂ or NR in compounds of Formula VI;
X is —C(O)—, —S(O)₂—, or a covalent bond;
Y is alkyl, alkenyl, cycloalkyl, alkylcycloalkyl, alkylcycloalkylalkyl, alkyloxyalkyl, aryl, alkylaryl, alkylarylalkyl, arylalkyl, cycloalkylalkyl, alkylheterocycle, heterocyclealkyl, alkylheterocyclealkyl, heterocycle, aminoalkyl, oxyalkyl, aminoaryl, oxyaryl;
Z is selected from the group consisting of —B(OR¹)OR², —CON(R¹)OR², and —N(OR¹)COR²;
R¹ and R² are each independently H, loweralkyl, or together form C₂-C₄ alkylene; and
R³, R⁴, R⁵, R⁶, and R⁷ and, if present, R, R⁸, R⁹, and R¹⁰ are each independently selected from the group consisting of: H, halo, loweralkyl, haloloweralkyl, haloloweralkoxy, loweralkoxy, hydroxy, loweralkoxycarbo, cycloalkyl, alkylcycloalkyl, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, nitro; arylalkyloxy, cycloalkyloxy, cycloalkylalkoxy, cycloalkylamino, urea, cycloalkylalkylamino, cycloalkyl, alkylcycloalkyl, hydroxyamino, alkoxyacylamino, and arylthio;
and 5- or 6-membered organic rings containing 0 to 4 heteroatoms selected from the group consisting of N, O and S, which rings may be unsubstituted or substituted from 1 to 4 times with halo, loweralkyl, haloloweralkyl, haloloweralkyloxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, nitro; and oxoheterocyclic groups;
or R⁸ and R⁹, if present, together are ═O or ═S;
or a pharmaceutically acceptable salt or prodrug thereof,
in an amount effective to treat the inflammatory disease.

13. The method of claim 12, wherein the compound is a compound of Formula I or Formula II and the compound is selected from the group consisting of:
4-(2-(Trifluoromethyl)-1H-benzo[d]imidazol-1-yl)butylboronic acid;
5-(2-(Thiazol-4-yl)-1H-benzo[d]imidazol-1-yl)pentylboronic acid;
5-(5,6-dimethyl-1H-benzo[d]imidazol-1-yl)pentylboronic acid;
5-(1H-imidazo[4,5-c]pyridin-1-yl)pentylboronic acid;
5-(2-(4-Methoxyphenyl)-1H-benzo[d]imidazol-1-yl)pentylboronic acid;
5-(2-(3-Fluoro-4-methoxyphenyl)-1H-benzo[d]imidazol-1-yl)pentylboronic acid;
5-(5-cyano-2-(4-methoxyphenyl)-1H-benzo[d]imidazol-1-yl)pentylboronic acid;
5-(6-cyano-2-(4-methoxyphenyl)-1H-benzo[d]imidazol-1-yl)pentylboronic acid;
and pharmaceutically acceptable salts and prodrugs thereof.

14. The method of claim 12, wherein the compound is a compound of Formula III, IV, or V and the compound is selected from the group consisting of:
5-(5-cyano-1H-indol-1-yl)pentylboronic acid;
and pharmaceutically acceptable salts and prodrugs thereof.

15. The method of claim 12, wherein the compound is a compound of Formula VI and the compound is selected from the group consisting of:
5-(6-fluoro-2,3-dihydro-3-oxobenzo[b][1,4]oxazin-4-yl)pentylboronic acid;
5-(2,3-dihydro-3-oxobenzo[b][1,4]thiazin-4-yl)pentylboronic acid;
5-(7-chloro-2,3-dihydro-3-oxobenzo[b][1,4]thiazin-4-yl)pentylboronic acid;
5-(2,3-dihydro-7-nitro-3-oxobenzo[b][1,4]oxazin-4-yl)pentylboronic acid;
5-(2,3-dihydro-3-oxobenzo[b][1,4]oxazin-4-yl)pentylboronic acid;
ethyl 2-(3,4-dihydro-3-oxo-4-(5-pentylboronic acid)-2H-benzo[b][1,4]thiazin-2-yl)acetate;
and pharmaceutically acceptable salts and prodrugs thereof.

16. The method of claim 12, wherein the inflammatory disease is selected from the group consisting of inflammatory bowel disease, rheumatoid arthritis, psoriasis, ankylosing spondylitis, psoriatic arthritis, asthma, chronic obstructive pulmonary disease, septic shock, allergic rhinitis, allergic conjunctivitis, atopic dermatitis, eczema, and Behcet's disease.

17. A method of treating a non-inflammatory disease in a subject in need thereof, the method comprising administering to the subject a compound selected from the group consisting of:
(a) a compound of Formula I or Formula II 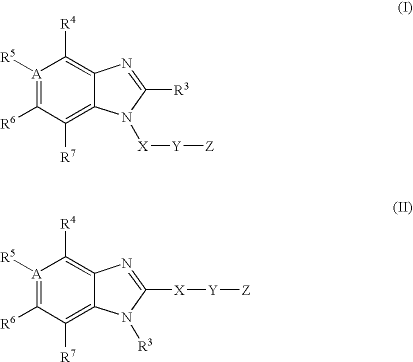
(b) a compound of Formula III, Formula IV, or Formula V: 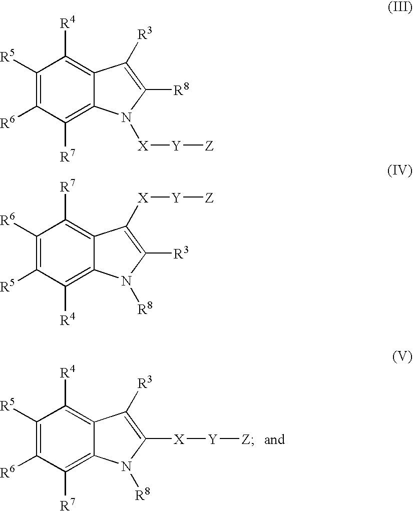
(c) a compound of Formula VI: 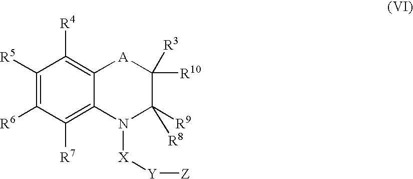
wherein:
A is N or C in compounds of Formula I and II, subject to the proviso that R⁵ is absent when A is N;
A is S, O, SO₂ or NR in compounds of Formula VI;
X is —C(O)—, —S(O)₂—, or a covalent bond;
Y is alkyl, alkenyl, cycloalkyl, alkylcycloalkyl, alkylcycloalkylalkyl, alkyloxyalkyl, aryl, alkylaryl, alkylarylalkyl, arylalkyl, cycloalkylalkyl, alkylheterocycle, heterocyclealkyl, alkylheterocyclealkyl, heterocycle, aminoalkyl, oxyalkyl, aminoaryl, oxyaryl;
Z is selected from the group consisting of —B(OR¹)OR², —CON(R¹)OR², and —N(OR¹)COR;
R¹ and R² are each independently H, loweralkyl, or together form C₂-C₄ alkylene; and
R³, R⁴, R⁵, R⁶, and R⁷ and, if present, R, R⁸, R⁹, and R¹⁰ are each independently selected from the group consisting of: H, halo, loweralkyl, haloloweralkyl, haloloweralkoxy, loweralkoxy, hydroxy, loweralkoxycarbo, cycloalkyl, alkylcycloalkyl, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, nitro; arylalkyloxy, cycloalkyloxy, cycloalkylalkoxy, cycloalkylamino, urea, cycloalkylalkylamino, cycloalkyl, alkylcycloalkyl, hydroxyamino, alkoxyacylamino, and arylthio;
and 5- or 6-membered organic rings containing 0 to 4 heteroatoms selected from the group consisting of N, O and S, which rings may be unsubstituted or substituted from 1 to 4 times with halo, loweralkyl, haloloweralkyl, haloloweralkyloxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, nitro; and oxoheterocyclic groups;
or R⁸ and R⁹, if present, together are ═O or ═S;
or a pharmaceutically acceptable salt or prodrug thereof;
in an amount effective to treat the non-inflammatory disease.

18. The method of claim 17, wherein the compound is a compound of Formula I or Formula II and the compound is selected from the group consisting of:
4-(2-(Trifluoromethyl)-1H-benzo[d]imidazol-1-yl)butylboronic acid;
5-(2-(Thiazol-4-yl)-1H-benzo[d]imidazol-1-yl)pentylboronic acid;
5-(5,6-dimethyl-1H-benzo[d]imidazol-1-yl)pentylboronic acid;
5-(1H-imidazo[4,5-c]pyridin-1-yl)pentylboronic acid;
5-(2-(4-Methoxyphenyl)-1H-benzo[d]imidazol-1-yl)pentylboronic acid;
5-(2-(3-Fluoro-4-methoxyphenyl)-1H-benzo[d]imidazol-1-yl)pentylboronic acid;
5-(5-cyano-2-(4-methoxyphenyl)-1H-benzo[d]imidazol-1-yl)pentylboronic acid;
5-(6-cyano-2-(4-methoxyphenyl)-1H-benzo[d]imidazol-1-yl)pentylboronic acid;
and pharmaceutically acceptable salts and prodrugs thereof.

19. The method of claim 17, wherein the compound is a compound of Formula III, IV, or V and the compound is selected from the group consisting of:
5-(5-cyano-1H-indol-1-yl)pentylboronic acid;
and pharmaceutically acceptable salts and prodrugs thereof.

20. The method of claim 17, wherein the compound is a compound of Formula VI and the compound is selected from the group consisting of:
5-(6-fluoro-2,3-dihydro-3-oxobenzo[b][1,4]oxazin-4-yl)pentylboronic acid;
5-(2,3-dihydro-3-oxobenzo[b][1,4]thiazin-4-yl)pentylboronic acid;
5-(7-chloro-2,3-dihydro-3-oxobenzo[b][1,4]thiazin-4-yl)pentylboronic acid;
5-(2,3-dihydro-7-nitro-3-oxobenzo[b][1,4]oxazin-4-yl)pentylboronic acid;
5-(2,3-dihydro-3-oxobenzo[b][1,4]oxazin-4-yl)pentylboronic acid;
ethyl 2-(3,4-dihydro-3-oxo-4-(5-pentylboronic acid)-2H-benzo[b][1,4]thiazin-2-yl)acetate;
and pharmaceutically acceptable salts and prodrugs thereof.

21. The method of claim 17, wherein the non-inflammatory disease is selected from the group consisting of Alzheimer's disease, type II diabetes, cancer, hypertension, and erectile dysfunction.
